(19) United States
(12) Patent Application Publication
(10) Pub. No.: US 2020/0399262 A1
(43) Pub. Date: Dec. 24, 2020

(54) COMPOUNDS FOR INHIBITING PROTEIN DEGRADATION AND METHODS OF USE THEREOF IN THE TREATMENT OF CANCER

(71) Applicant: PI Therapeutics Ltd, Ness Ziona (IL)

(72) Inventors: Ori KALID, Pardes Hanna-Karkur (IL); Irina GOTLIV, Rehovot (IL); Einat LEVY-APTER, Rehovot (IL); Danit FINKELSHTEIN BEKER, Tel Aviv (IL); Parkash JAGTAP, North Andover, MA (US)

(73) Assignee: PI Therapeutics Ltd, Ness Ziona (IL)

(21) Appl. No.: 16/978,697

(22) PCT Filed: Mar. 7, 2019
(86) PCT No.: PCT/IL2019/050250

§ 371 Date: Sep. 6, 2020

Related U.S. Application Data
(60) Provisional application No. 62/640,298, filed on Mar. 8, 2018.
(60) Provisional application No. 62/640,263, filed on Mar. 8, 2018.
(60) Provisional application No. 62/712,713, filed on Jul. 31, 2018.
(60) Provisional application No. 62/712,692, filed on Jul. 31, 2018.

(51) Int. Cl. C07D 413/14; A61P 35/00; C07D 213/68; C07D 213/89

(52) U.S. Cl. CPC C07D 413/14; C07D 213/89; C07D 213/68; A61P 35/00

(57) ABSTRACT

The present invention relates to compounds for inhibiting protein degradation and/or the ubiquitin-proteasome system and/or for modulating autophagy, pharmaceutical composition and methods of use thereof in the treatment of cancer.

### TECHNICAL FIELD

The present invention relates to compounds for inhibiting protein degradation and/or the ubiquitin-proteasome system and/or for modulating autophagy, pharmaceutical composition and methods of use thereof in the treatment of cancer.

### BACKGROUND

Cancer is the second most common cause of death in the United States accounting for 1 of every 4 deaths. From 2000 through 2009, death rates from all cancers combined decreased on average 1.8% per year among men and 1.4% per year among women. This improvement in survival reflects progress in early diagnosis and treatment. Discovering highly effective anticancer agents with low toxicity is a primary goal of cancer research (Cancer Facts & Figures American Cancer Society: Atlanta, Ga. (2008)).

Malignant cells harbor genomic aberrations such as copy number alterations, aneuploidy, and mutations, which can exacerbate misfolded and unfolded protein burden, resulting in increased deleterious proteotoxic stress. For that, malignant cells rely heavily on the protein quality control mechanisms of the cell for survival and proliferation. (John H. Van Drie, Chin J Cancer. 2011 February; 30(2): 124-137).

Protein homeostasis is maintained by a well-controlled balance between synthesis and degradation of proteins. The UPS is the major protein degradation pathway in the cell. Proteins destined to degradation by the UPS are tagged by conjugation to ubiquitin, through the action of ubiquitin-conjugating ligases, resulting in ubiquitin chains on one or more lysine residues within the substrate that mark them for degradation. Endoplasmic reticulum (ER) is the organelle responsible for synthesis, folding, and structural maturation of proteins in the cell, therefore it is an important component regulating protein homeostasis. Under normal conditions, incompletely folded proteins are retro-translocated back to the cytosol and degraded by the proteasome in a process known as ER-associated degradation (ERAD) (Deshaies BMC Biology 2014, 12:94). When misfolded proteins in the ER accumulate above a critical threshold, a signal transduction pathway, called the unfolded protein response (UPR) is initiated, enabling cells to mitigate the problem by inhibiting protein synthesis to reduce the load on the ER, while upregulating genes to enhance the biogenic capacity of the ER. However, sustained UPR signaling can eventually commit a cell to apoptosis (Scott A. Am J Physiol Cell Physiol. 2017 Feb. 1; 312(2): C93-C102). [Deshaies BMC Biology 2014, 12:94].

Another mechanism contributing to protein homeostasis and cell health is autophagy. The autophagy pathway, among its many functions, contributes to the clearance of misfolded or aggregated proteins through lysosomal degradation. (Danielle Glick et al J Pathol. Author manuscript; available in PMC 2010 Nov. 23.) Recently, autophagy has been acknowledged as an important mechanism controlling multiple aspects of cancer biology (Naiara Santana-Codina, Joseph D. Mancias,1, and Alec C. Kimmelman Annual Review of Cancer Biology Vol. 1:19-39 (Volume publication date March 2017).

The UPS, UPR and autophagy, are all under tight and complex regulation, orchestrating a cascade of events that allow the cells to cope with proteotoxic stress. Dependency of malignant cells on these components mark them as attractive targets in cancer therapeutics.

The plasma cell disorders are a spectrum of conditions including asymptomatic precursor states such as monoclonal gammopathy of undetermined significance (MGUS) and smoldering multiple myeloma (SMM), symptomatic malignancies such as multiple myeloma (MM) and Waldenstrom's macroglobulinemia (WM) and disorders such as immunoglobulin light chain (AL) amyloidosis and POEMS syndrome. Plasma cell disorders are characterized by a high rate of abnormal immunoglobulin production associated with ongoing proteotoxic stress and high baseline induction of UPR (Cenci S, Sitia R. FEBS Lett. 2007; 581(19):3652-3657). This molecular characteristic highlights the therapeutic potential of compounds that disrupt the protein homeostasis machinery.

In evidence, proteasome inhibition is an established treatment strategy for patients with multiple myeloma (MM). MM is a clonal plasma cell disorder characterized by uncontrolled proliferation and bone marrow infiltration of aberrant plasma cells, which secrets abnormal monoclonal proteins. It is the second most common hematologic malignancy in the United States with 30,770 estimated new cases in 2018 (1.8% of all new cancer cases in the US) accounting for 12,770 estimated deaths in the US in 2018 (2.1% of all cancer deaths) (https://seer.cancer.gov/statfacts/html/mulmy.html). MM is an aggressive and incurable disease for most patients, characterized by periods of treatment, remission and relapse, in which patients face increasingly worse outcomes. Subsequent line of therapy results in a shorter duration of response accompanied with an increased risk of treatment and disease-related complications. Poor prognosis in relapse stages reflects genomic complexity of tumors acquiring multiple genetic and epigenetic alterations that promote treatment resistance and refractory disease (R F Cornell and A A Kassim Bone Marrow Transplant. 2016 April; 51(4): 479-491). First line of therapy for MM patients includes the proteasome inhibitor (PI) bortezomib (BTZ), which demonstrated remarkable response rates. By inhibiting the proteasome, BTZ causes accumulation of misfolded protein in the endoplasmic reticulum (ER) and activation of the unfolded protein response (UPR), which in turn leads to cell apoptosis (from: chari et al. biologics 4, 273-287, 2010). In recent years, additional MM drugs have been developed that target protein homeostasis including 2^{nd}-generation PIs (carfilzomib and ixazomib) and histone deacetylase inhibitors.

The 2^{nd} generation PI carfilzomib has also shown promise as frontline treatment for another malignant plasma cell disorder, Waldenstrom's macroglobulinemia (WM), a rare incurable disease characterized by the infiltration of the bone marrow by clonal lymphoplasmacytic cells and a monoclonal immunoglobulin M (IgM) gammopathy in the blood (Leuk Lymphoma. 2018 Sep. 19:1-7).

Non-plasma-cell hematologic malignancies are also responsive to treatment with PIs. There include Mantle cell lymphoma (MCL), a B-cell non-Hodgkin's lymphoma (NHL) where bortezomib is approved for treatment of newly diagnosed as well as relapsed refractory disease, and ALL (Br J Haematol. 2017 February; 176(4):629-636; Blood 2012 120:285-290).

Additional hematologic conditions treated successfully with PIs include AL Amyloidosis and post-transplant lymphoproliferative disease (PTLD). AL Amyloidosis, characterized by deposition of amyloid fibrils derived from light chain immunoglobulins produced by monoclonal plasma cells, has been treated successfully with bortezomib (Merlini G, Bellotti V. Molecular mechanisms of amyloidosis. N Engl J Med 2003; 349:583-96.). PTLD, a lymphoproliferative disorder secondary to chronic immunosuppression, has been successfully treated with a combination of bortezomib and dexamethasone, based on multiple myeloma protocols [Pediatr Blood Cancer 2013; 60:E137-E139].

In addition to hematologic disorders and malignancies, agents disrupting protein homeostasis may also be useful for the treatment of various solid tumors. These include SMARCB1-deficient malignancies, demonstrated to exhibit dramatic activation of the UPR and ER stress response via the MYC-p19ARF-p53 axis (Cancer Cell 35, 204-220, Feb. 11, 2019) as well as additional tumor types.

### SUMMARY OF INVENTION

It has been found by the inventors of the subject application that compounds described by the invention induce proteotoxic stress and UPR by modulating protein degradation pathways and disrupting protein homeostasis, suggesting that these compound may be effective therapeutic options for plasma cell disorders such as MM, WM, plasma cell leukemia, plasmacytoma, AL amyloidosis and PTLD, other hematologic malignancies such MCL and also solid tumor indication involving protein homeostasis dependency such as SMARCB1-deficient tumors.

Accordingly, in various embodiments, this invention is directed to a compound represented by the structure of Formula IV:

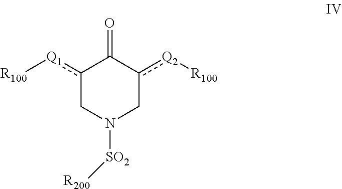

or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof; wherein Q₁, Q₂, R₁₀₀ and R₂₀₀ are as defined herein below.

In other embodiments, R₁₀₀ is a substituted phenyl or a substituted 5 or 6 membered monocylclic heteroaryl (e.g., isoxazole). In other embodiments, R₁₀₀ is substituted with at least one selected from: CH₃, F, Cl, NO₂, CF₃ or CN. In other embodiments, R₁₀₀ is an aryl represented by the structure of formula V:

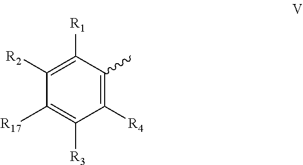

wherein R₁, R₂, R₃, R₄ and R₁₇ are as defined herein below. In other embodiments, R₁₇ is CN, Cl or F and R₂ is Cl, CF₃ or H. In other embodiments, R₂₀₀ is R₁₅—N(R₁₃)(R₁₄), R₁₅—O(R₁₃), R₁₅—Cl, or R₁₅—Br. In other embodiments, R₁₅ is (CH₂)₂ or (CH₂)₃, R₁₃ is CH₃, and R₁₄ is CH₃ or a C₁-C₁₄ linear alkyl group substituted with C₁-C₁₄ linear or branched alkynyl or N₃. In other embodiments, the compound is represented by the structure of compounds D1, AA, CA, E1, BA, F1, A2, BA-2, A3, CA-2, F1-5, E1-2 or AA-8 as defined herein above.

In various embodiments, this invention is directed to a compound represented by the structure of Formula III:

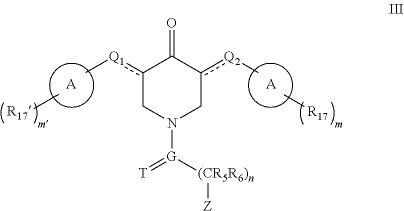

or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof; wherein A, Q₁, Q₂, R₅, R₆, n, R₁₇, R₁₇′ m, m′, G, T, G=T and Z are as defined herein below.

In other embodiments, A is a phenyl or an isoxazole. In other embodiments, m and m′ are each independently 1 or 2, and R₁₇ and R₁₇′ are each independently H, F, Cl, Br, I, CN, CH₃, CF₃ or NO₂. In other embodiments, Q₁ is CH and Q₂ is CH or CH₂. In other embodiments, R₅ and R₆ are each independently H, OH, R₁₅—OH, CH₂—OH, COOH, C₁-C₁₀ alkyl, iPr, OR₁₃, OMe, NH₂, N(R₁₃)(R₁₄), N(CH₃)₂, or R₅ and R₆ are joint to form a substituted or unsubstituted (C₃-C₈) cycloalkyl, a cyclopropyl, a substituted or unsubstituted (C₃-C₈) heterocyclic ring, or a morpholine. In other embodiments, G is C and T is O, or G=T is SO₂. In other embodiments, R₁₃ is H, OH, methyl, methoxyethyl, phenyl, pyridyl, or C(O)—CH₃, and R₁₄ is H, or methyl. In other embodiments, the compound is represented by the structure of compounds AA, B1-B3, B6-B30, B32, BA, C1, D1, E1, F1, H1, B1-11, B2-7, C1-7, or C1-8 as defined herein above.

In some embodiments, this invention is directed to a compound represented by the structure of Formula II:

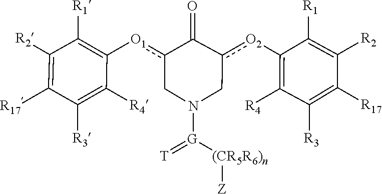

or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof; wherein Q₁, Q₂, R₁, R₂, R₃, R₄, R₁′, R₂′, R₃′, R₄′, R₅, R₆, n, R₁₇, R₁₇′, G, T and Z are as defined herein below.

In other embodiments, R₁₇ and R₁₇′ are each independently Cl, CN, H, or F; R₂ and R₂′ are each independently H, CF₃, CN, Cl or NO₂; and R₄ and R₄′ are each independently H or Cl. In other embodiments, G is C and T is O, or G=T is SO₂. In other embodiments, the compound is represented by the structure of compounds AA, B1-B32, BA, CA, C1, D1, G1, H1, B1-11, B2-7, C1-7, or C1-8 as defined herein above.

In some embodiments, this invention is directed to a compound represented by the structure of Formula I:

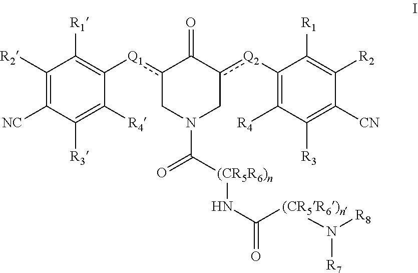

or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof; wherein Q₁, Q₂, R₁, R₂, R₃, R₄, R₁′, R₂′, R₃′, R₄′, R₅, R₆, R₅′, R₆′, R₇, R₈, n, and n′ are as defined herein below.

In other embodiments, R₇ and R₈ are each independently substituted or unsubstituted linear or branched C₁-C₁₀ alkyl, a methyl, a propyl azide or a propynyl. In other embodiments, R₁, R₂, R₃, R₁′, R₂′, R₃′, and R₄′ are H. In other embodiments, R₅, R₆, R₅′ and R₆′ are H. In other embodiments, Q₁ is CH and Q₂ is CH or CH₂. In other embodiments, R₇ is a methyl, C₃ alkyl substituted with N₃ or CH₂—C≡CH, and R₈ is a methyl. In other embodiments, the compound is represented by the structure of compound B1-B3, C1, G1, or H1 as defined herein above.

In other embodiments, the compound is a protein degradation inhibitor, a UPS inhibitor, an autophagy modulator, a UPR inducer or any combination thereof. In other embodiments, the compound induces proteotoxic stress and UPR by modulating protein degradation pathways and disrupting protein homeostasis, the compound induces accumulation of poly-ubiquitinated proteins in cells treated therewith, the compound disrupts autophagosomal flux in cells treated therewith, the compound induces the unfolded protein response (UPR) in cells treated therewith or any combination thereof.

In various embodiments, this invention is directed to a pharmaceutical composition comprising the compound of this invention, and a pharmaceutically acceptable carrier.

In various embodiments, this invention is directed to a method of treating, suppressing, reducing the severity, reducing the risk of developing or inhibiting cancer comprising administering a compound according to any one of the preceding claims to a subject suffering from cancer under conditions effective to treat, suppress, reduce the severity, reduce the risk of developing, or inhibit said cancer. In other embodiments, the cancer is selected from the list of: multiple myeloma, leukemia, Alveolar rhabdomyosarcoma, Melanoma, lymphoma, Astrocytoma, Biphasic synovial sarcoma, Bladder carcinoma, Bone cancer Breast Cancer, Cecum adenocarcinoma, Cervical cancer, CNS cancer, Colon cancer, Colorectal cancer, Duodenal adenocarcinoma, Embryonal rhabdomyosarcoma, Endometrial cancer, Epithelioid sarcoma, Fibrosarcoma, Gastric cancer, Signet ring cell gastric adenocarcinoma, Gestational choriocarcinoma, Glioblastoma, Hereditary thyroid gland medullary carcinoma, Hypopharyngeal squamous cell carcinoma, Invasive ductal carcinoma, Liposarcoma, Lung cancer, Neuroblastoma, Osteosarcoma, Ovarian cancer, Uterine cancer, Pancreatic cancer, Papillary renal cell carcinoma, Prostate cancer, Rectal adenocarcinoma, Medulloblastoma, Renal cancer, Testicular embryonal carcinoma and Tongue squamous cell carcinoma; each represents a separate embodiment according to this invention. In some embodiments, the cancer is early cancer, advanced cancer, invasive cancer, metastatic cancer, drug resistant cancer or any combination thereof; each represents a separate embodiment according to this invention. In some embodiments, the subject has been previously treated with chemotherapy, immunotherapy, radiotherapy, biological therapy, surgical intervention, or any combination thereof; each represents a separate embodiment according to this invention. In some embodiments, the compound is administered in combination with an anti-cancer therapy. In some embodiments, the anti-cancer therapy is chemotherapy, immunotherapy, radiotherapy, biological therapy, surgical intervention, or any combination thereof; each represents a separate embodiment according to this invention.

In various embodiments, this invention is directed to a method of suppressing, reducing or inhibiting tumor growth in a subject, comprising administering a compound according to this invention, to a subject suffering from cancer under conditions effective to suppress, reduce or inhibit said tumor growth in said subject. In some embodiments, the tumor is a solid tumor. In some embodiments, the tumor is a SMARCB1-deficient tumor.

In various embodiments, this invention is directed to a method of treating, suppressing, reducing the severity, reducing the risk of developing or inhibiting a plasma cell disorder comprising administering a compound according to this invention to a subject suffering from plasma cell disorder under conditions effective to treat, suppress, reduce the severity, reduce the risk of developing, or inhibit said plasma cell disorder. In some embodiments, the plasma cell disorder is Monoclonal Gammopathy of Undetermined Significance (MGUS), smoldering multiple myeloma (SMM), Asymptomatic Plasma Cell Myeloma, Multiple myeloma (MM), Waldenstrom's macroglobulinemia (WM), immunoglobulin light chain (AL) amyloidosis, POEMS syndrome, plasma cell (PC) leukemia, or Plasmacytoma; each represents a separate embodiment according to this invention. In some embodiments, the plasma cell disorder is malignant.

In various embodiments, this invention is directed to a method of treating, suppressing, reducing the severity, reducing the risk of developing or inhibiting a Non-plasma-cell hematologic malignancy in a subject, comprising administering a compound according to this invention to a subject suffering from Non-plasma-cell hematologic malignancy under conditions effective to treat, suppress, reduce the severity, reduce the risk of developing, or inhibit said Non-plasma-cell hematologic malignancy. In some embodiments, the Non-plasma-cell hematologic malignancy is B-cell non-Hodgkin's lymphoma (NHL) such as Mantle cell lymphoma (MCL).

In various embodiments, this invention is directed to a method of treating, suppressing, reducing the severity, reducing the risk of developing or inhibiting a hematologic condition comprising administering a compound according to this invention to a subject suffering from hematologic condition under conditions effective to treat, suppress, reduce the severity, reduce the risk of developing, or inhibit said hematologic condition. In some embodiments, the hematologic condition is AL Amyloidosis, post-transplant lymphoproliferative disease (PTLD) or combination thereof; each represents a separate embodiment according to this invention.

In various embodiments, this invention is directed to a method of treating, suppressing, reducing the severity, reducing the risk of developing or inhibiting a SMARCB1-deficient malignancy in a subject, comprising administering a compound according to this invention to a subject suffering from a SMARCB1-deficient malignancy under conditions effective to treat, suppress, reduce the severity, reduce the risk of developing, or inhibit said SMARCB1-deficient malignancy.

In various embodiments, this invention is directed to a method of treating, suppressing, reducing the severity, reducing the risk of developing or inhibiting a Post-transplant lymphoproliferative disease (PTLD) comprising administering a compound according to this invention to a subject suffering from Post-transplant lymphoproliferative disease (PTLD) under conditions effective to treat, suppress, reduce the severity, reduce the risk of developing, or inhibit said Post-transplant lymphoproliferative disease (P TLD). In some embodiments, the PTLD is polymorphic PTLD, monomorphic PTLD or classical Hodgkin-lymphoma-type PTLD; each represents a separate embodiment according to this invention.

In various embodiments, this invention is directed to a method of treating, suppressing, reducing the severity, reducing the risk of developing or inhibiting multiple myeloma comprising administering a compound according to this invention to a subject suffering from multiple myeloma under conditions effective to treat, suppress, reduce the severity, reduce the risk of developing, or inhibit said multiple myeloma.

### BRIEF DESCRIPTION OF DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

FIG. 1A-1C show that Compound B1 (FIG. 1A), Compound AA (FIG. 1B) and compound E1 (FIG. 1C) induce the accumulation of poly-ubiquinated proteins according to some embodiments of the present invention. MM1.S cells were treated with Compound B1 (FIG. 1A), Compound AA (FIG. 1B) and Compound E1 (FIG. 1C) for indicated periods of time. Following treatment, the cells were harvested, and the lysates resolved on SDS-PAGE. Transferred membranes were blotted with antibodies as indicated. Actin was used as loading control.

FIG. 2 shows that Compound B1 and compound AA do not inhibit the enzymatic functions of the proteasome according to some embodiments of the present invention. Proteasome activity was measured in intact MM1.S cells as cleavage of peptide substrates, specific for Trypsin like (TL), Chemotrypsin like (CTL) and Caspase like (PL) activities of the proteasome following treatment with Compound B1, Compound AA or Bortezomib (BTZ) at ˜EC₅₀ concentrations for 3 hr at 37° C. BTZ was used as positive control.

FIG. 3A-3B depict the kinetic solubility of Compound B1 (FIG. 3A) and Compound E1 (FIG. 3B) as measured by differential UV absorbance of the compounds, as performed before and after centrifugation. Soluble concentrations were determined when OD was equivalent between centrifuged and non-centrifuged fractions. Compounds were dissolved from co-solvent stock, and further serially 2-fold diluted in PBS. OD was measured at the maximal absorbance for each compound before (BC) and after (AC) centrifugation, using Spark 20M, Tecan.

FIG. 4A-4D depicts the growth inhibition of MM1.S xenograft in nude mice by Compound B1 (FIG. 4A, FIG. 4B) and Compound AA (FIG. 4C, FIG. 4D). FIG. 4A and FIG. 4C show tumor growth inhibition observed at end point measurements by Compound B1 and AA respectively. FIG. 4B and FIG. 4D shows the body weight % changes in animals treated with Compound B1 and Compound AA respectively. No significant weight loss was observed in mice treated with Compound B1 and Compound AA at 5 mg/kg and 4 mg/kg respectively. MM.1S cells (5×10⁶ cells/mouse) were implanted in the rear flank of mail mice (6 weeks of age at the time of tumor implantation). On Day 20-23, mice were randomized for equivalent distribution of tumor volumes to treatment groups (n=5/group) and treated IV with vehicle, compound B1 (FIG. 4A) and compound AA (FIG. 4C), three time a week (TIW) for 21 days. Data are presented as mean tumor volume±SD. Body weight % changes in treated animals observed during the course of the study for compound B1 (FIG. 4B) and Compound AA (FIG. 4D).

FIG. 5 depicts the in-vitro safety of Compound B1 and Compound AA in Peripheral Blood Mononuclear Cells (PBMCs) from healthy donors respectively. MM1.S cells and normal PBMCs from healthy donors were treated with various concentrations of indicated compounds for 6 h and then analyzed 48 h later for cell viability (ATPlight assay). Compound B1 and Compound AA were less cytotoxic to PBMCs from healthy donors than Ixazomib, Bortezomib (BTZ) and CB5083. Calculated therapeutic windows: EC₅₀ (MM1.S)/EC₅₀ (PBMCs), generated from 5 healthy donor PBMC samples, based on mean viability data.

FIG. 6A-6D show the evaluated in-vivo efficacy of Compound AA in a colorectal mouse flank xenograft models (HCT116, SW620). Treatment of tumor-bearing mice with Compound AA significantly inhibited tumor growth at 8 mg/kg compared to vehicle control in both xenograft models (FIG. 6A and FIG. 6B). Animal body weight was not considerably affected by the treatment (FIG. 6C, FIG. 6D). HCT-116 or SW620 cells (5×10⁶ cells/mouse) were implanted in the rear flank of mail mice (6 weeks of age at the time of tumor implantation). On Day 20-23, mice were randomized for equivalent distribution of tumor volumes to treatment groups (n=5/group) and treated IV with vehicle, Compound AA (FIG. 6A, FIG. 6B) TIW for 21 days. Data are presented as mean tumor volume±SD. Body weight % changes in treated animals observed at end point (FIG. 6C, FIG. 6D).

FIG. 7A-7K depict an immunoblot analysis of UPR in cells treatment with Compound B1 demonstrating activation of all UPR branches (PERK, ATF6 and IRE1alpha). MM1.S cells were treated with 200 nM of Compound B1 for the indicated time points. Following the stated incubation periods the cells were harvested, lysed and resolved on SDS-PAGE gel. Proteins were transferred to PVDF membrane and immunoblotted with the indicated antibodies: FIG. 7A: anti phospho JNK, FIG. 7B: anti JNK, FIG. 7C: anti ATF6. FIG. 7D: anti phospho eIF2alpha, FIG. 7E: anti eIF2alpha, FIG. 7F: anti ATF4. XBP1 splicing was performed on cDNA (FIG. 7G), RNA was extracted cDNA was generated by RT-PCR and XPB1 transcript was amplified by PCR with gene specific primers. Splicing was detected by differential migration of XBP1 transcript on agarose gel. Transcriptional changes of CHOP (FIG. 7K) and ATF4 (FIG. 7J) were estimated by quantitative PCR with gene specific primers. Relative gene expression levels were normalized to GAPDH. Cleaved form of ATF6 and spliced XBP1 is indicated with arrow.

FIG. 8 depicts autophagy modulation following treatment with Compound B1, suggesting disruption of autophagosomal flux. MM1.S cells were treated with 0.2 μM Compound B1 or vehicle (DMSO) for 5 h. Detection of autophagy vesicles was done by CYTO-ID® green autophagy dye that selectively labels autophagic vacuoles. The samples were analyzed using flow cytometer and the data were plotted on histogram: cell counts vs. FITC fluorescence intensity.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

### DETAILED DESCRIPTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

The UPS is central to the regulation of almost all cellular processes including: antigen processing, apoptosis, biogenesis of organelles, cell cycle and division, DNA transcription and repair, differentiation and development, immune response and inflammation, neural and muscular degeneration, morphogenesis of neural networks, modulation of cell surface receptors, ion channels and the secretory pathway, response to stress and extracellular modulators, ribosome biogenesis, and viral infection.

Specific degradation of a protein via the UPS involves two discrete and successive steps: tagging of the substrate protein by the covalent attachment of multiple ubiquitin molecules (Conjugation); and the subsequent degradation of the tagged protein by the 26S proteasome, composed of the catalytic 20S core and the 19S regulator multi-subunit heterocomplexes (Degradation). This classical function of ubiquitin is associated with housekeeping functions, regulation of protein turnover and antigenic-peptide generation.

The compounds according to this invention, are in some embodiments, inhibitors of the Ubiquitin Proteasome System (UPS). In some embodiments, the compounds according to this invention are inhibitors of protein degradation. In some embodiments, the compounds according to this invention disrupt autophagosomal flux in cells treated therewith. In some embodiments, the compounds according to this invention induce accumulation of poly-ubiquitinated proteins in cells treated therewith. In some embodiments, the compounds according to this invention induce the unfolded protein response (UPR) in cells treated therewith.

In some embodiments, the present invention relates to a compound of formula (I):

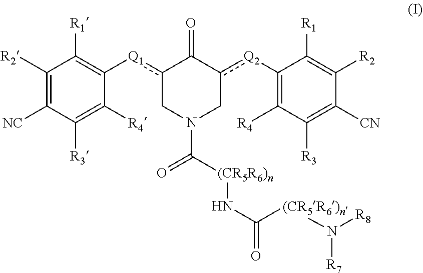

wherein
Q₁ and Q₂ are each independently, either CH or CH₂;
R₁, R₂, R₃, R₄, R₁′, R₂′, R₃′ and R₄′ are each, independently, selected from:
   H, NO₂, OH, COOH, NH₂, F, Cl, Br, I, CN, R₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₅, R₆, R₅′ and R₆′ are each, independently, selected from: H, F, Cl, Br, I, OH, R₁₅—OH (e.g., CH₂—OH), COOH, CN, C₁-C₁₀ alkyl (e.g., iPr), OR₁₃ (e.g., OMe), NH₂, N(R₁₃)(R₁₄) (e.g., N(CH₃)₂), substituted or unsubstituted (C₃-C₈) cycloalkyl, substituted or unsubstituted (C₃-C₈) heterocyclic ring having one or more heteroatoms selected from N, O and S; or R₅ and R₆ are joint to form a substituted or unsubstituted (C₃-C₈) cycloalkyl (e.g., cyclopropyl) or a substituted or unsubstituted (C₃-C₈) heterocyclic ring (e.g. morpholine); or R₅′ and R₆′ are joint to form a substituted or unsubstituted (C₃-C₈) cycloalkyl or a substituted or unsubstituted (C₃-C₈) heterocyclic ring; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₇ and R₈ are each independently selected from: H, F, Cl, Br, I, substituted or unsubstituted linear or branched C₁-C₁₀ alkyl (e.g. methyl, ethyl, propyl, iso-propyl, butyl, sec-butyl, tert-butyl), substituted or unsubstituted linear or branched C₁-C₁₀ alkoxy, substituted or unsubstituted aryl, substituted or unsubstituted heteroaryl, C(O)—R₁₃, S(O)—R₁₃, S(O)₂—R₁₃, R₁₅-Ph, R₁₅-aryl, R₁₅-heteroaryl, R₁₅-R₁₃, R₁₅-R₁₆-R₁₃ (e.g., CH₂—C≡CH, —CH₂—CH═CH—C₁-C₁₀ alkyl, —CH₂—CH═CH₂, substituted or unsubstituted (C₃-C₈) cycloalkyl, substituted or unsubstituted (C₃-C₈) heterocyclic ring having one or more heteroatoms selected from N, O and S; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q1}R₁₃; and
R₁₃ and R₁₄ are each independently selected from: H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), —C(O)—C1-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl (e.g. CH₂—C≡CH), aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₅ is [CH₂]ₚ
   wherein p is between 1 and 10;
R₁₆ is [CH]q, [C]
   wherein q is between 2 and 10; and
n and n′ are each independently an integer between 1 and 15;
   or a geometrical isomer, optical isomer, solvate, metabolite, pharmaceutically sacceptable salt, pharmaceutical product, tautomer, hydrate, N-oxide, prodrug, isotopic variant (e.g., deuterated analog), PROTAC, polymorph, or crystal thereof.

In some embodiments Q₁ and Q₂ are both CH. In some embodiments Q₁ is CH and Q₂ is CH₂. In some embodiments Q₁ and Q₂ are both CH₂.

In some embodiments R₁, R₂, R₃, and R₄ are the same as R₁′, R₂′, R₃′, and R₄′ respectively. In some embodiments R₁, R₂, R₃, R₄ and R₁′, R₂′, R₃′, and R₄′ are each independently H. In some embodiments R₁, R₂, R₃, R₄ and R₁′, R₂′, R₃′, and R₄′ are all H. In some embodiments R₁, R₂, R₃, R₄ and R₁′, R₂′, R₃′, and R₄′ are each independently H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, or S(O)₂R₁₃; each is a separate embodiment according to this invention. In some embodiments R₂ and R₂′ are Cl. In some embodiments R₂ and R₂′ are F. In some embodiments R₂ and R₂′ are Br. In some embodiments R₂ and R₂′ are I. In some embodiments R₂ and R₂′ are CN. In some embodiments R₂ and R₂′ are NO₂. In some embodiments R₂ and R₂′ are CF₃.

In some embodiments R₅ and R₆ are the same. In some embodiments R₅ and R₆ are both H. In some embodiments R₅ and R₆ are both C₁-C₁₀ alkyl. In some embodiments R₅′ and R₆′ are the same. In some embodiments R₅′ and R₆′ are both H. In some embodiments R₅′ and R₆′ are both C₁-C₁₀ alkyl. In some embodiments R₅, R₆, R₅′ and R₆′ are each independently H. In some embodiments, R₅, R₆, R₅′ and R₆′ are each independently C₁-C₁₀ alkyl. In some embodiments, R₅, R₆, R₅′ and R₆′ are each independently methyl. In some embodiments, R₅, R₆, R₅′ and R₆′ are each independently R₁₅—OH. In some embodiments, R₅ is H and R₆ is R₁₅—OH. In some embodiments, R₅′ is H and R₆′ is R₁₅—OH.

In some embodiments R₅, R₆, R₅′ and R₆′ are each independently F. In some embodiments, R₅, R₆, R₅′ and R₆′ are each independently selected from: H, F, Cl, Br, I, OH, R₁₅—OH (e.g., CH₂—OH), COOH, CN, C₁-C₁₀ alkyl (e.g., iPr), OR₁₃ (e.g., OMe), NH₂, N(R₁₃)(R₁₄) (e.g., N(CH₃)₂), substituted or unsubstituted (C₃-C₈) cycloalkyl, substituted or unsubstituted (C₃-C₈) heterocyclic ring having one or more heteroatoms selected from N, O and S; each represents a separated embodiment according to this invention. In some embodiments, the substitutions are at least one of: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃; each represents a separated embodiment according to this invention. In some embodiments R₅, R₆, R₅′ and R₆′ are each independently H. In some embodiments R₅, R₆, R₅′ and R₆′ are each independently OH. In some embodiments R₅, R₆, R₅′ and R₆′ are each independently R₁₅—OH. In some embodiments R₅, R₆, R₅′ and R₆′ are each independently CH₂—OH. In some embodiments R₅, R₆, R₅′ and R₆′ are each independently COOH. In some embodiments R₅, R₆, R₅′ and R₆′ are each independently C₁-C₁₀ alkyl. In some embodiments R₅, R₆, R₅′ and R₆′ are each independently iPr. In some embodiments R₅, R₆, R₅′ and R₆′ are each independently OR₁₃. In some embodiments R₅, R₆, R₅′ and R₆′ are each independently OMe. In some embodiments R₅, R₆, R₅′ and R₆′ are each independently NH₂. In some embodiments R₅, R₆, R₅′ and R₆′ are each independently N(R₁₃)(R₁₄). In some embodiments R₅, R₆, R₅′ and R₆′ are each independently N(CH₃)₂. In some embodiments, R₅ and R₆ are joint to form a substituted or unsubstituted (C₃-C₈) cycloalkyl. In some embodiments, R₅ and R₆ are joint to form a cyclopropyl. In some embodiments, R₅ and R₆ are joint to form a substituted or unsubstituted (C₃-C₈) heterocyclic ring. In some embodiments, R₅ and R₆ are joint to form a morpholine ring. In some embodiments, R₅′ and R₆′ are joint to form a substituted or unsubstituted (C₃-C₈) cycloalkyl. In some embodiments, R₅′ and R₆′ are joint to form a substituted or unsubstituted (C₃-C₈) heterocyclic ring.

In some embodiments, R₇ and R₈ are different. In some embodiments, R₇ and R₈ are the same. In some embodiments, R₇ and R₈ are each independently H, F, Cl, Br, I, substituted or unsubstituted linear or branched C₁-C₁₀ alkyl (e.g. methyl, ethyl, propyl, iso-propyl, butyl, sec-butyl, tert-butyl), substituted or unsubstituted linear or branched C₁-C₁₀ alkoxy, substituted or unsubstituted aryl, substituted or unsubstituted heteroaryl, C(O)—R₁₃, S(O)—R₁₃, S(O)₂—R₁₃, R₁₅-Ph, R₁₅-aryl, R₁₅-heteroaryl, R₁₅-R₁₃, R₁₅-R₁₆-R₁₃ (e.g., CH₂—C≡CH, —CH₂—CH═CH—C₁-C₁₀ alkyl, —CH₂—CH═CH₂, substituted or unsubstituted (C₃-C₈) cycloalkyl, substituted or unsubstituted (C₃-C₈) heterocyclic ring having one or more heteroatoms selected from N, O and S; wherein substitutions are selected from: C₁-C₁₄ linear or branched haloalkyl, C₁-C₁₄ linear or branched alkoxy, C₁-C₁₄ linear or branched alkenyl, C₁-C₁₄ linear or branched alkynyl, NO₂, OH, OR₁₃, COOH, NH₂, C₁-C₁₄ alkylamino, C1-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)₁R₁₃; each is a separate embodiment according to this invention. In some embodiments, R₇ and R₈ are different. In some embodiments, R₇ and R₈ are the same. In some embodiments, R₇ and R₈ are each independently a substituted or unsubstituted linear or branched C₁-C₁₀ alkyl. In some embodiments R₇ and R₈ are each independently H. In some embodiments, R₇ and R₈ are each independently a methyl. In some embodiments, R₇ and R₈ are both a methyl. In some embodiments, R₇ and R₈ are each independently an ethyl, a propyl, an iso-propyl, a butyl, an iso-butyl, a tert-butyl, a pentyl; each is a separate embodiment according to this invention. In some embodiments, R₇ is an ethyl, a propyl, an iso-propyl, a butyl, an iso-butyl, a tert-butyl, a pentyl and R₈ is a methyl; each is a separate embodiment according to this invention. In some embodiments, R₇ is an ethyl, a propyl, an iso-propyl, a butyl, an iso-butyl, a tert-butyl, a pentyl and R₈ is H; each is a separate embodiment according to this invention. In some embodiments, R₇ and R₈ are each independently an C₁-C₁₀ alkyl substituted with N₃. In some embodiments, R₇ and R₈ are each independently a C₃ alkyl substituted with N₃. In some embodiments, R₇ is a C₃ alkyl substituted with N₃ and R₈ is a methyl. In some embodiments, R₇ and R₈ are each independently a R₁₅-R₁₆-R₁₃. In some embodiments, R₇ and R₈ are each independently CH₂—C≡CH. In some embodiments, R₇ is CH₂—C≡CH and R₈ is a methyl. In some embodiments, R₇ and R₈ are each independently a substituted or unsubstituted aryl. In some embodiments, R₇ and R₈ are each independently a substituted or unsubstituted heteroaryl. In some embodiments, R₇ and R₈ are each independently C(O)—CH₃. In some embodiments, R₇ and R₈ are each independently S(O)₂—CH₃. In some embodiments, R₇ and R₈ are each independently R₁₅-aryl. In some embodiments, R₇ is R₁₅-R₁₆-R₁₃, and R₁₅ is CH₂, R₁₆ is [C]_{q}, q is 2 and R₁₃ is H.

In some embodiments, R₁₃ and R₁₄ are different. In some embodiments, R₁₃ and R₁₄ are the same. In some embodiments, R₁₃ and R₁₄ are each independently H, Cl, Br, F, I, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, halogen, N₃, and CN; each is a separate embodiment according to this invention. In some embodiments, R₁₃ and R₁₄ are each independently H. In some embodiments, R₁₃ and R₁₄ are each independently a methyl. In some embodiments, R₁₃ and R₁₄ are each independently methoxyethyl. In some embodiments, R₁₃ and R₁₄ are each independently substituted or unsubstituted aryl. In some embodiments, R₁₃ and R₁₄ are each independently phenyl. In some embodiments, R₁₃ and R₁₄ are each independently substituted or unsubstituted heteroaryl. In some embodiments, R₁₃ and R₁₄ are each independently pyridyl. In some embodiments, R₁₃ and R₁₄ are each independently C(O)—CH₃. In some embodiments, R₁₃ is H. In some embodiments, R₁₃ and R₁₄ are each independently —C(O)—C₁-C₁₄ substituted or unsubstituted linear or branched alkyl, In some embodiments, R₁₃ and R₁₄ are each independently —C(O)—CH₃. In some embodiments, R₁₃ and R₁₄ are each independently OH. In some embodiments, R₁₃ and R₁₄ are each independently a substituted or unsubstituted C₁-C₁₄ linear or branched alkyl group. In some embodiments, R₁₃ is methyl. In some embodiments, R₁₃ and R₁₄ are each independently a substituted C₁-C₁₄ linear or branched alkyl group, substituted with N₃. In some embodiments, R₁₃ and R₁₄ are each independently a substituted C₁-C₁₄ linear or branched alkyl group, substituted with C₁-C₁₄ linear or branched alkynyl. In some embodiments, R₁₃ and R₁₄ are each independently substituted with C₁-C₁₄ linear or branched alkoxy. In some embodiments, R₁₃ and R₁₄ are each independently substituted with C₁-C₁₄ linear or branched methoxy. In some embodiments, R₁₃ and R₁₄ are each independently C(O)—C₁-C₁₄ linear or branched alkyl. In some embodiments, R₁₃ and R₁₄ are each independently C₁-C₁₄ linear or branched-S(O)₂-alkyl. In some embodiments, R₁₃ and R₁₄ are each independently Cl. In some embodiments, R₁₃ and R₁₄ are each independently Br. In some embodiments, R₁₃ and R₁₄ are each independently I. In some embodiments, R₁₃ and R₁₄ are each independently F.

In some embodiments, R₁₅ is CH₂. In some embodiments, R₁₅ is [CH₂]₂. In some embodiments, R₁₅ is [CH₂]₃. In some embodiments, R₁₅ is [CH₂]_{q}.

In some embodiment, p is 1. In some embodiment, p is 2. In some embodiment, p is 3. In some embodiment, p is 4. In some embodiment, p is 5. In some embodiment, p is 6. In some embodiment, p is 7.

In some embodiments, R₁₆ is [CH]_{q}. In some embodiments, R₁₆ is [C]_{q}.

In some embodiments, q is 2. In some embodiments, q is 3. In some embodiments, q is 4. In some embodiments, q is 5. In some embodiments, q is 6.

In some embodiment, n of compound of Formula I is 1. In some embodiment, n is 2. In some embodiment, n is 3. In some embodiment, n is 4. In some embodiment, n is 5. In some embodiment, n is 6. In some embodiment, n is 7.

In some embodiment, n′ is 1. In some embodiment, n′ is 2. In some embodiment, n′ is 3. In some embodiment, n′ is 4. In some embodiment, n′ is 5. In some embodiment, n′ is 6. In some embodiment, n′ is 7.

In some embodiments, R₇ is R₁₅-R₁₆-R₁₃, and R₁₅ is CH₂, R₁ is [C]g, q is 2 and R₁₃ is H.

In some embodiments, compounds of Formula (I) are represented by the structures of Compounds B1, B2, B3, Cl, G1 and H1 as described herein below; each represents a separate embodiment according to this invention.

In some embodiments, the present invention relates to a compound, represented by the structure of Formula II:

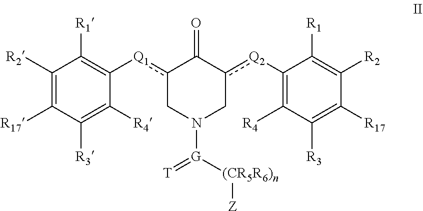

wherein
Q₁ and Q₂ are each independently, either CH or CH₂;
R₁, R₂, R₃, R₄ R₁′, R₂′, R₃′, and R₄′ are each, independently, selected from:
   H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C1-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₅, and R₆ are each, independently, selected from: H, F, Cl, Br, I, OH, R₁₅—OH (e.g., CH₂—OH), COOH, CN, C₁-C₁₀ alkyl (e.g., iPr), OR₁₃ (e.g., OMe), NH₂, N(R₁₃)(R₁₄) (e.g., N(CH₃)₂), substituted or unsubstituted (C₃-C₈) cycloalkyl, substituted or unsubstituted (C₃-C₈) heterocyclic ring having one or more heteroatoms selected from N, O and S; or R₅ and R are joint to form a substituted or unsubstituted (C₃-C₈) cycloalkyl (e.g., cyclopropyl) or a substituted or unsubstituted (C₃-C₈) heterocyclic ring (e.g. morpholine); wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₇ and R₈ are each independently selected from: H, F, Cl, Br, I, substituted or unsubstituted linear or branched C₁-C₁₀ alkyl (e.g. methyl, ethyl, propyl, iso-propyl, butyl, sec-butyl, tert-butyl), substituted or unsubstituted linear or branched C₁-C₁₀ alkoxy, substituted or unsubstituted aryl, substituted or unsubstituted heteroaryl, C(O)—R₁₃, S(O)—R₁₃, S(O)₂—R₁₃, R₁₅-Ph, R₁₅-aryl, R₁₅-heteroaryl, R₁₅-R₁₃, R₁₅-R₁₆-R₁₃ (e.g., CH₂—C≡CH, —CH₂—CH═CH—C₁-C₁₀ alkyl, —CH₂—CH═CH₂, substituted or unsubstituted (C₃-C₈) cycloalkyl, substituted or unsubstituted (C₃-C₈) heterocyclic ring having one or more heteroatoms selected from N, O and S; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q1}R₁₃;
R₁₃ and R₁₄ are each independently selected from: H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C1-C₁₄ linear or branched alkenyl, C₁-C₁₄ linear or branched alkynyl (e.g. CH₂—C≡CH), aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₅ is [CH₂]ₚ
   wherein p is between 1 and 10;
R₁₆ is [CH]_{q}, [C]_{q}
   wherein q is between 2 and 10;
n is an integer between 1 and 15;
R₁₇ and R₁₇′ are each independently selected from H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
G is C, S or N;
T is O, S, NH, N—OH, CH₂, CR₁₃R₁₄; or
G=T is SO₂; and
Z is H, —NH—C(O)—R₁₅—N(R₇)(R₈), F, Cl, Br, I, N(R₁₃)(R₁₄) (e.g., N(Me)₂, NH(COMe), NH₂), OR₁₃ (e.g., OMe), —NH—C(O)—R₁₅-R₁₃, substituted or unsubstituted aryl (e.g., phenyl), substituted or unsubstituted heteroaryl, substituted or unsubstituted R₁₅-aryl (e.g., benzyl, CH₂-phenyl-OH), substituted or unsubstituted R₁₅-heteroaryl (e.g., CH₂-pyridyl), C(O)—NH—R₁₃ (e.g., C(O)—NH—CH₃); or geometrical isomer, optical isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal thereof.

In some embodiments R₁₇ is the same as R₁₇′. In some embodiments, R₁₇ and R₁₇′ are each independently H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, or S(O)₂R₁₃; each represent a separate embodiment according to this invention. In some embodiments R₁₇, and R₁₇′ are each independently H. In some embodiments R₁₇, and R₁₇′ are each independently Cl. In some embodiments R₁₇, and R₁₇′ are each independently F. In some embodiments R₁₇, and R₁₇′ are each independently Br. In some embodiments R₁₇, and R₁₇′ are each independently I. In some embodiments R₁₇, and R₁₇′ are each independently CN. In some embodiments R₁₇, and R₁₇′ are each independently NO₂.

In some embodiments G is C. In some embodiments G is S. In some embodiments G is N.

In some embodiments T is O. In some embodiments T is S. In some embodiments T is NH. In some embodiments T is N—OH. In some embodiments T is CH₂. In some embodiments T is CR₁₃R₁₄.

In some embodiments G=T is SO₂.

In some embodiments, Z is H. In some embodiments, Z is —NH—C(O)—R₁₅—N(R₇)(R₈). In some embodiments, Z is F. In some embodiments, Z is Cl. In some embodiments, Z is Br. In some embodiments, Z is I. In some embodiments, Z is N(R₁₃)(R₁₄). In some embodiments, Z is N(Me)₂. In some embodiments, Z is NH(COMe). In some embodiments, Z is NH₂. In some embodiments, Z is OR₁₃. In some embodiments, Z is OMe. In some embodiments, Z is —NH—C(O)—R₁₅-R₁₃. In some embodiments, Z is substituted or unsubstituted aryl. In some embodiments, Z is phenyl. In some embodiments, Z is substituted or unsubstituted heteroaryl. In some embodiments, Z is substituted or unsubstituted R₁₅-aryl. In some embodiments, Z is benzyl. In some embodiments, Z is CH₂-phenyl-OH. In some embodiments, Z is substituted or unsubstituted R₁₅-heteroaryl. In some embodiments, Z is CH₂-pyridyl. In some embodiments, Z is C(O)—NH—R₁₃. In some embodiments, Z is C(O)—NH—CH₃.

In some embodiments R₁, R₂, R₃, and R₄ are the same as R₁′, R₂′, R₃′, and R₄′ respectively. In some embodiments R₁, R₂, R₃, R₄ and R₁′, R₂′, R₃′, and R₄′ are H. In some embodiments R₁, R₂, R₃, R₄ and R₁′, R₂′, R₃′, and R₄′ are each independently H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C1-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, or S(O)₂R₁₃; each is a separate embodiment according to this invention. In some embodiments R₂ and R₂′ are Cl. In some embodiments R₄′ and R₂′ are Cl. In some embodiments R₂ and R₂′ are F. In some embodiments R₂ and R₂′ are Br. In some embodiments R₂ and R₂′ are I. In some embodiments R₂ and R₂′ are CN. In some embodiments R₂ and R₂′ are NO₂. In some embodiments R₂ and R₂′ are CF₃.

In some embodiments Q₁ and Q₂ are both CH. In some embodiments Q₁ is CH and Q₂ is CH₂. In some embodiments Q₁ and Q₂ are both CH₂.

In some embodiments R₅ and R₆ are the same. In some embodiments R₅ and R₆ are each independently F. In some embodiments, R₅ and R₆ are each independently selected from: H, F, Cl, Br, I, OH, R₁₅—OH (e.g., CH₂—OH), COOH, CN, C₁-C₁₀ alkyl (e.g., iPr), OR₁₃ (e.g., OMe), NH₂, N(R₁₃)(R₁₄) (e.g., N(CH₃)₂), substituted or unsubstituted (C₃-C₈) cycloalkyl, substituted or unsubstituted (C₃-C₈) heterocyclic ring having one or more heteroatoms selected from N, O and S; each represents a separated embodiment according to this invention. In some embodiments, the substitutions are at least one of: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C1-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃; each represents a separated embodiment according to this invention. In some embodiments R₅ and R₆ are each independently OH. In some embodiments R₅ and R₆ are each independently R₁₅—OH. In some embodiments R₅ and R₆ are each independently CH₂—OH. In some embodiments R₅ and R₆ are each independently COOH. In some embodiments R₅ and R₆ are each independently C₁-C₁₀ alkyl. In some embodiments R₅ and R₆ are both C₁-C₁₀ alkyl. In some embodiments R₅ and R₆ are each independently iPr. In some embodiments, R₅ and R₆ are each independently methyl. In some embodiments R₅ and R₆ are each independently OR₁₃. In some embodiments R₅ and R₆ are each independently OMe. In some embodiments R₅ and R₆ are each independently NH₂. In some embodiments R₅ and R₆ are each independently N(R₁₃)(R₁₄). In some embodiments R₅ and R₆ are each independently N(CH₃)₂. In some embodiments, R₅ and R₆ are joint to form a substituted or unsubstituted (C₃-C₈) cycloalkyl. In some embodiments, R₅ and R₆ are joint to form a cyclopropyl. In some embodiments, R₅ and R₆ are joint to form a substituted or unsubstituted (C₃-C₈) heterocyclic ring. In some embodiments, R₅ and R₆ are joint to form a morpholine ring. In some embodiments R₅ and R₆ are both H. In some embodiments R₅ and R₆ are each independently H. In some embodiments, R₅ is H and R₆ is R₁₅—OH.

In some embodiments, R₇ and R₈ are different. In some embodiments, R₇ and R₈ are the same. In some embodiments R₇ and R₈ are each independently H. In some embodiments, R₇ and R₈ are each independently a substituted or unsubstituted linear or branched C₁-C₁₀ alkyl. In some embodiments, R₇ and R₈ are each independently a methyl. In some embodiments, R₇ and R₈ are both a methyl. In some embodiments, R₇ and R₈ are each independently an ethyl, a propyl, an iso-propyl, a butyl, an iso-butyl, a tert-butyl, a pentyl; each is a separate embodiment according to this invention. In some embodiments, R₇ is an ethyl, a propyl, an iso-propyl, a butyl, an iso-butyl, a tert-butyl, a pentyl and R₈ is a methyl; each is a separate embodiment according to this invention. In some embodiments, R₇ is an ethyl, a propyl, an iso-propyl, a butyl, an iso-butyl, a tert-butyl, a pentyl and R₈ is H; each is a separate embodiment according to this invention. In some embodiments, R₇ and R₈ are each independently a substituted C₁-C₁₀ alkyl. In some embodiments, R₇ and R₈ are each independently an C₁-C₁₀ alkyl substituted with N₃. In some embodiments, R₇ and R₈ are each independently a C₃ alkyl substituted with N₃. In some embodiments, R₇ is a C₃ alkyl substituted with N₃ and R₈ is a methyl. In some embodiments, R₇ and R₈ are each independently a R₁₅-R₁₆-R₁₃. In some embodiments, R₇ is R₁₅-R₁₆-R₁₃, and R₁₅ is CH₂, R₁₆ is [C]q, q is 2 and R₁₃ is H. In some embodiments, R₇ and R₈ are each independently CH₂—C≡CH. In some embodiments, R₇ is CH₂—C≡CH and R₈ is a methyl. In some embodiments, R₇ and R₈ are each independently a substituted or unsubstituted aryl. In some embodiments, R₇ and R₈ are each independently a substituted or unsubstituted heteroaryl. In some embodiments, R₇ and R₈ are each independently substituted with at least one selected from: C₁-C₁₄ linear or branched haloalkyl, C1-C₁₄ linear or branched alkoxy, C1-C₁₄ linear or branched alkenyl, C₁-C₁₄ linear or branched alkynyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q}R₁₃; each is a separate embodiment according to this invention. In some embodiments, R₇ and R₈ are each independently C(O)—CH₃. In some embodiments, R₇ and R₈ are each independently S(O)₂—CH₃. In some embodiments, R₇ and R₈ are each independently R₁₅-aryl.

In some embodiments, R₁₃ and R₁₄ are different. In some embodiments, R₁₃ and R₁₄ are the same. In some embodiments, R₁₃ and R₁₄ are each independently H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), OH, —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁—C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, halogen, N₃, and CN; each is a separate embodiment according to this invention. In some embodiments, R₁₃ and R₁₄ are each independently H. In some embodiments, R₁₃ and R₁₄ are each independently a methyl. In some embodiments, R₁₃ and R₁₄ are each independently methoxyethyl. In some embodiments, R₁₃ and R₁₄ are each independently substituted or unsubstituted aryl. In some embodiments, R₁₃ and R₁₄ are each independently phenyl. In some embodiments, R₁₃ and R₁₄ are each independently substituted or unsubstituted heteroaryl. In some embodiments, R₁₃ and R₁₄ are each independently pyridyl. In some embodiments, R₁₃ and R₁₄ are each independently C(O)—CH₃. In some embodiments, R₁₃ is H. In some embodiments, R₁₃ and R₁₄ are each independently —C(O)—C₁-C₁₄ substituted or unsubstituted linear or branched alkyl, In some embodiments, R₁₃ and R₁₄ are each independently —C(O)—CH₃. In some embodiments, R₁₃ and R₁₄ are each independently OH. In some embodiments, R₁₃ and R₁₄ are each independently a substituted or unsubstituted C₁-C₁₄ linear or branched alkyl group. In some embodiments, R₁₃ and R₁₄ are each independently a substituted C₁-C₁₄ linear or branched alkyl group, substituted with N₃. In some embodiments, R₁₃ and R₁₄ are each independently a substituted C₁-C₁₄ linear or branched alkyl group, substituted with C₁-C₁₄ linear or branched alkynyl. In some embodiments, R₁₃ and R₁₄ are each independently substituted with C₁-C₁₄ linear or branched alkoxy. In some embodiments, R₁₃ and R₁₄ are each independently substituted with C₁-C₁₄ linear or branched methoxy. In some embodiments, R₁₃ is methyl. In some embodiments, R₁₃ and R₁₄ are each independently C(O)—C₁-C₁₄ linear or branched alkyl. In some embodiments, R₁₃ and R₁₄ are each independently C₁-C₁₄ linear or branched-S(O)₂-alkyl. In some embodiments, R₁₃ and R₁₄ are each independently Cl. In some embodiments, R₁₃ and R₁₄ are each independently Br. In some embodiments, R₁₃ and R₁₄ are each independently I. In some embodiments, R₁₃ and R₁₄ are each independently F.

In some embodiments, R₁₅ is CH₂. In some embodiments, R₁₅ is [CH₂]₂. In some embodiments, R₁₅ is [CH₂]₃. In some embodiments, R₁₅ is [CH₂]₄.

In some embodiment, p is 1. In some embodiment, p is 2. In some embodiment, p is 3. In some embodiment, p is 4. In some embodiment, p is 5. In some embodiment, p is 6. In some embodiment, p is 7.

In some embodiments, R₁₆ is [CH]_{q}. In some embodiments, R₁₆ is [C]_{q}.

In some embodiments, q is 2. In some embodiments, q is 3. In some embodiments, q is 4. In some embodiments, q is 5. In some embodiments, q is 6.

In some embodiment, n is 1. In some embodiment, n is 2. In some embodiment, n is 3. In some embodiment, n is 4. In some embodiment, n is 5. In some embodiment, n is 6. In some embodiment, n is 7.

In some embodiments, R₇ is R₁₅-R₁₆-R₁₃, and R₁₅ is CH, R₁ is [C], q is 2 and R₁ n is H.

In some embodiments, the compounds of Formula (II) are represented by the structures of Compounds AA, BA, CA, B1, B2, B3, B4, B5, B6, B7, B8, B9, B10, B11, B12, B13, B14, B15, B16, B17, B18, B19, B20, B21, B22, B23, B24, B25, B26, B27, B28, B29, B30, B31, B32, C1, D1, F1, G1, H1, B1-11, C1-7, C1-8, or B2-7, as described herein below; each represents a separate embodiment according to this invention.

In some embodiments, the present invention relates to a compound, represented by the structure of Formula III:

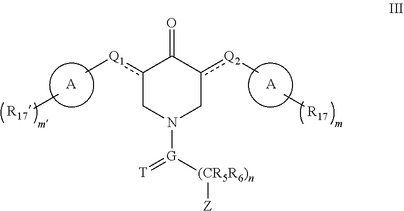

wherein
A ring is a single or fused aromatic or heteroaromatic ring system (e.g., phenyl, isoxazole, oxazole, 2-, 3- or 4-pyridine, benzofuran, benzo[d][1,3]dioxole, naphthalene, thiophene, thiazole, benzimidazole, piperidine, imidazole, diazole, triazole, tetrazole, isoquinoline), or a single or fused C₃-C₁₀ cycloalkyl (e.g. cyclohexyl) or a single or fused C₃-C₁₀ heterocyclic ring;
Q₁ and Q₂ are each independently, either CH or CH₂;
R₅, and R₆ are each, independently, selected from: H, F, Cl, Br, I, OH, R₁₅—OH (e.g., CH₂—OH), COOH, CN, C₁-C₁₀ alkyl (e.g., iPr), OR₁₃ (e.g., OMe), NH₂, N(R₁₃)(R₁₄) (e.g., N(CH₃)₂), substituted or unsubstituted (C₃-C₈) cycloalkyl, substituted or unsubstituted (C₃-C₈) heterocyclic ring having one or more heteroatoms selected from N, O and S; or R₅ and R are joint to form a substituted or unsubstituted (C₃-C₈) cycloalkyl (e.g., cyclopropyl) or a substituted or unsubstituted (C₃-C₈) heterocyclic ring (e.g. morpholine); wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁—C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₅) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₇ and R₈ are each independently selected from: H, F, Cl, Br, I, substituted or unsubstituted linear or branched C₁-C₁₀ alkyl (e.g. methyl, ethyl, propyl, iso-propyl, butyl, sec-butyl, tert-butyl), substituted or unsubstituted linear or branched C₁-C₁₀ alkoxy, substituted or unsubstituted aryl, substituted or unsubstituted heteroaryl, C(O)—R₁₃, S(O)—R₁₃, S(O)₂—R₁₃, R₁₅-Ph, R₁₅-aryl, R₁₅-heteroaryl, R₁₅-R₁₃, R₁₅—R₁₆-R₁₃ (e.g., CH₂—C≡CH, —CH₂—CH═CH—C₁-C₁₀ alkyl, —CH₂—CH═CH₂, substituted or unsubstituted (C₃-C₈) cycloalkyl, substituted or unsubstituted (C₃-C₈) heterocyclic ring having one or more heteroatoms selected from N, O and S; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q1}R₁₃; and
R₁₃ and R₁₄ are each independently selected from: H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), OH, —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, halogen, N₃, and CN;
R₁₅ is [CH₂]ₚ
   wherein p is between 1 and 10;
R₁₆ is [CH]_{q}, [C]_{q}
   wherein q is between 2 and 10;
n is an integer between 1 and 15;
R₁₇ and R₁₇′ are each independently selected from H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C1-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
m and m′ are each independently an integer between 0 and 5;
G is C, S or N;
T is O, S, NH, N—OH, CH₂, CR₁₃R₁₄; or
G=T is SO₂; and
Z is H, —NH—C(O)—R₁₅—N(R₇)(R₈), F, Cl, Br, I, N(R₁₃)(R₁₄) (e.g., N(Me)₂, NH(COMe), NH₂), OR₁₃ (e.g., OMe), —NH—C(O)—R₁₅-R₁₃, substituted or unsubstituted aryl (e.g., phenyl), substituted or unsubstituted heteroaryl, substituted or unsubstituted R₁₅-aryl (e.g., benzyl, CH₂-phenyl-OH), substituted or unsubstituted R₁₅-heteroaryl (e.g., CH₂-pyridyl), C(O)—NH—R₁₃ (e.g., C(O)—NH—CH₃); or geometrical isomer, optical isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal thereof.

In some embodiments, A ring is a single or fused aromatic or heteroaromatic ring system. In some embodiments, A ring is a phenyl. In some embodiments, A ring is an isoxazole. In some embodiments, A ring is a oxazole. In some embodiments, A ring is 2-, 3- or 4-pyridine. In some embodiments, A ring is a benzofuran. In some embodiments, A ring is a benzo[d][1,3]dioxole. In some embodiments, A ring is a naphthalene. In some embodiments, A ring is a thiophene. In some embodiments, A ring is a thiazole. In some embodiments, A ring is a benzimidazole. In some embodiments, A ring is a piperidine. In some embodiments, A ring is a imidazole. In some embodiments, A ring is a diazole. In some embodiments, A ring is a triazole. In some embodiments, A ring is a tetrazole. In some embodiments, A ring is a isoquinoline. In some embodiments, A ring is a single or fused C₃-C₁ cycloalkyl. In some embodiments, A ring is a cyclohexyl. In some embodiments, A ring is a single or fused C₃-C₁ heterocyclic ring.

In some embodiments, R₁₇ and R₁₇′ are each independently H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, or S(O)₂R₁₃; each represent a separate embodiment according to this invention. In some embodiments R₁₇, and R₁₇′ are each independently H. In some embodiments, R₁₇ is the same as R₁₇′. In some embodiments R₁₇, and R₁₇′ are each independently H. In some embodiments R₁₇, and R₁₇′ are each independently Cl. In some embodiments R₁₇, and R₁₇′ are each independently F. In some embodiments R₁₇, and R₁₇′ are each independently Br. In some embodiments R₁₇, and R₁₇′ are each independently I. In some embodiments R₁₇, and R₁₇′ are each independently methyl. In some embodiments R₁₇, and R₁₇′ are each independently F. In some embodiments R₁₇, and R₁₇′ are each independently Br. In some embodiments R₁₇, and R₁₇′ are each independently I. In some embodiments R₁₇, and R₁₇′ are each independently CN. In some embodiments R₁₇, and R₁₇′ are each independently NO₂.

In some embodiments G is C. In some embodiments G is S. In some embodiments G is N.

In some embodiments T is O. In some embodiments T is S. In some embodiments T is NH. In some embodiments T is N—OH. In some embodiments T is CH₂. In some embodiments T is CR₁₃R₁₄.

In some embodiments G=T is SO₂.

In some embodiments, Z is H. In some embodiments, Z is —NH—C(O)—R₁₅—N(R₇)(R₈). In some embodiments, Z is F. In some embodiments, Z is Cl. In some embodiments, Z is Br. In some embodiments, Z is I. In some embodiments, Z is N(R₁₃)(R₁₄). In some embodiments, Z is N(Me)₂. In some embodiments, Z is NH(COMe). In some embodiments, Z is NH₂. In some embodiments, Z is OR₁₃. In some embodiments, Z is OMe. In some embodiments, Z is —NH—C(O)—R₁₅-R₁₃. In some embodiments, Z is substituted or unsubstituted aryl. In some embodiments, Z is phenyl. In some embodiments, Z is substituted or unsubstituted heteroaryl. In some embodiments, Z is substituted or unsubstituted R₁₅-aryl. In some embodiments, Z is benzyl. In some embodiments, Z is CH₂-phenyl-OH. In some embodiments, Z is substituted or unsubstituted R₁₅-heteroaryl. In some embodiments, Z is CH₂-pyridyl. In some embodiments, Z is C(O)—NH—R₁₃. In some embodiments, Z is C(O)—NH—CH₃.

In some embodiments Q₁ and Q₂ are both CH. In some embodiments Q₁ is CH and Q₂ is CH₂. In some embodiments Q₁ and Q₂ are both CH₂.

In some embodiments R₅ and R₆ are the same. In some embodiments R₅ and R₆ are each independently F. In some embodiments, R₅ and R₆ are each independently selected from: H, F, Cl, Br, I, OH, R₁₅—OH (e.g., CH₂—OH), COOH, CN, C₁-C₁₀ alkyl (e.g., iPr), OR₁₃ (e.g., OMe), NH₂, N(R₁₃)(R₁₄) (e.g., N(CH₃)₂), substituted or unsubstituted (C₃-C₈) cycloalkyl, substituted or unsubstituted (C₃-C₈) heterocyclic ring having one or more heteroatoms selected from N, O and S; each represents a separated embodiment according to this invention. In some embodiments, the substitutions are at least one of: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃; each represents a separated embodiment according to this invention. In some embodiments R₅ and R₆ are each independently OH. In some embodiments R₅ and R₆ are each independently R₁₅—OH. In some embodiments R₅ and R₆ are each independently CH₂—OH. In some embodiments R₅ and R₆ are each independently COOH. In some embodiments R₅ and R₆ are each independently C₁-C₁₀ alkyl. In some embodiments R₅ and R₆ are both C₁-C₁₀ alkyl. In some embodiments R₅ and R₆ are each independently iPr. In some embodiments, R₅ and R₆ are each independently methyl. In some embodiments R₅ and R₆ are each independently OR₁₃. In some embodiments R₅ and R₆ are each independently OMe. In some embodiments R₅ and R₆ are each independently NH₂. In some embodiments R₅ and R₆ are each independently N(R₁₃)(R₁₄). In some embodiments R₅ and R₆ are each independently N(CH₃)₂. In some embodiments, R₅ and R₆ are joint to form a substituted or unsubstituted (C₃-C₈) cycloalkyl. In some embodiments, R₅ and R₆ are joint to form a cyclopropyl. In some embodiments, R₅ and R₆ are joint to form a substituted or unsubstituted (C₃-C₈) heterocyclic ring. In some embodiments, R₅ and R₆ are joint to form a morpholine ring. In some embodiments R₅ and R₆ are both H. In some embodiments R₅ and R₆ are each independently H. In some embodiments, R₅ is H and R₆ is R₁₅—OH.

In some embodiments, R₇ and R₈ are different. In some embodiments, R₇ and R₈ are the same. In some embodiments R₇ and R₈ are each independently H. In some embodiments, R₇ and R₈ are each independently a substituted or unsubstituted linear or branched C₁-C₁₀ alkyl. In some embodiments, R₇ and R₈ are each independently a methyl. In some embodiments, R₇ and R₈ are both a methyl. In some embodiments, R₇ and R₈ are each independently an ethyl, a propyl, an iso-propyl, a butyl, an iso-butyl, a tert-butyl, a pentyl; each is a separate embodiment according to this invention. In some embodiments, R₇ is an ethyl, a propyl, an iso-propyl, a butyl, an iso-butyl, a tert-butyl, a pentyl and R₈ is a methyl; each is a separate embodiment according to this invention. In some embodiments, R₇ is an ethyl, a propyl, an iso-propyl, a butyl, an iso-butyl, a tert-butyl, a pentyl and R₈ is H; each is a separate embodiment according to this invention. In some embodiments, R₇ and R₈ are each independently a substituted C₁-C₁₀ alkyl. In some embodiments, R₇ and R₈ are each independently an C₁-C₁₀ alkyl substituted with N₃. In some embodiments, R₇ and R₈ are each independently a C₃ alkyl substituted with N₃. In some embodiments, R₇ is a C₃ alkyl substituted with N₃ and R₈ is a methyl. In some embodiments, R₇ and R₈ are each independently a R₁₅-R₁₆-R₁₃. In some embodiments, R₇ and R₈ are each independently CH₂—C≡CH. In some embodiments, R₇ is CH₂—C≡CH and R₈ is a methyl. In some embodiments, R₇ and R₈ are each independently a substituted or unsubstituted aryl. In some embodiments, R₇ and R₈ are each independently a substituted or unsubstituted heteroaryl. In some embodiments, R₇ and R₈ are each independently substituted with at least one selected from: C₁-C₁₄ linear or branched haloalkyl, C₁-C₁₄ linear or branched alkoxy, C₁-C₁₄ linear or branched alkenyl, C₁-C₁₄ linear or branched alkynyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q1}R₁₃; each is a separate embodiment according to this invention. In some embodiments, R₇ and R₈ are each independently C(O)—CH₃. In some embodiments, R₇ and R₈ are each independently S(O)₂—CH₃. In some embodiments, R₇ and R₈ are each independently R₁₅-aryl.

In some embodiments, R₁₃ and R₁₄ are different. In some embodiments, R₁₃ and R₁₄ are the same. In some embodiments, R₁₃ and R₁₄ are each independently H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), OH, —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, halogen, N₃, and CN; each is a separate embodiment according to this invention. In some embodiments, R₁₃ and R₁₄ are each independently H. In some embodiments, R₁₃ and R₁₄ are each independently a methyl. In some embodiments, R₁₃ and R₁₄ are each independently methoxyethyl. In some embodiments, R₁₃ and R₁₄ are each independently substituted or unsubstituted aryl. In some embodiments, R₁₃ and R₁₄ are each independently phenyl. In some embodiments, R₁₃ and R₁₄ are each independently substituted or unsubstituted heteroaryl. In some embodiments, R₁₃ and R₁₄ are each independently pyridyl. In some embodiments, R₁₃ and R₁₄ are each independently C(O)—CH₃. In some embodiments, R₁₃ is H. In some embodiments, R₁₃ and R₁₄ are each independently —C(O)—C₁-C₁₄ substituted or unsubstituted linear or branched alkyl, In some embodiments, R₁₃ and R₁₄ are each independently —C(O)—CH₃. In some embodiments, R₁₃ and R₁₄ are each independently OH. In some embodiments, R₁₃ and R₁₄ are each independently a substituted or unsubstituted C₁-C₁₄ linear or branched alkyl group. In some embodiments, R₁₃ and R₁₄ are each independently a substituted C₁-C₁₄ linear or branched alkyl group, substituted with N₃. In some embodiments, R₁₃ and R₁₄ are each independently a substituted C₁-C₁₄ linear or branched alkyl group, substituted with C₁-C₁₄ linear or branched alkynyl. In some embodiments, R₁₃ and R₁₄ are each independently substituted with C₁-C₁₄ linear or branched alkoxy. In some embodiments, R₁₃ and R₁₄ are each independently substituted with C₁-C₁₄ linear or branched methoxy. In some embodiments, R₁₃ is methyl. In some embodiments, R₁₃ and R₁₄ are each independently C(O)—C₁-C₁₄ linear or branched alkyl. In some embodiments, R₁₃ and R₁₄ are each independently C₁-C₁₄ linear or branched-S(O)₂-alkyl. In some embodiments, R₁₃ and R₁₄ are each independently Cl. In some embodiments, R₁₃ and R₁₄ are each independently Br. In some embodiments, R₁₃ and R₁₄ are each independently I. In some embodiments, R₁₃ and R₁₄ are each independently F.

In some embodiments, R₁₅ is CH₂. In some embodiments, R₁₅ is [CH₂]₂. In some embodiments, R₁₅ is [CH₂]₃. In some embodiments, R₁₅ is [CH₂]₄.

In some embodiment, p is 1. In some embodiment, p is 2. In some embodiment, p is 3. In some embodiment, p is 4. In some embodiment, p is 5. In some embodiment, p is 6. In some embodiment, p is 7.

In some embodiments, R₁₆ is [CH]_{q}. In some embodiments, R₁₆ is [C]_{q}.

In some embodiments, q is 2. In some embodiments, q is 3. In some embodiments, q is 4. In some embodiments, q is 5. In some embodiments, q is 6.

In some embodiment, n is 1. In some embodiment, n is 2. In some embodiment, n is 3. In some embodiment, n is 4. In some embodiment, n is 5. In some embodiment, n is 6. In some embodiment, n is 7.

In some embodiment, m is 0. In some embodiment, m is 1. In some embodiment, m is 2. In some embodiment, m is 3. In some embodiment, m is 4.

In some embodiment, m′ is 0. In some embodiment, m′ is 1. In some embodiment, m′ is 2. In some embodiment, m′ is 3. In some embodiment, m′ is 4.

In some embodiments, R₇ is R₁₅-R₁₆-R₁₃, and R₁₅ is CH₂, R₁₆ is [C]g, q is 2 and R₁₃ is H.

In some embodiments, a compound of Formula (III) is represented by the structure of Compound AA, BA, B1, B2, B3, B6, B7, B8, B9, B10, B11, B12, B13, B14, B15, B16, B17, B18, B19, B20, B21, B22, B23, B24, B25, B26, B27, B28, B29, B30, B32, C1, D1, E1, F1, G1, H1, B1-11, C1-7, C1-8, or B2-7 as described herein below; or a geometrical isomer, optical isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal thereof; each represents a separate embodiment according to this invention.

In some embodiments, the present invention relates to a compound, represented by the structure of Compound A:

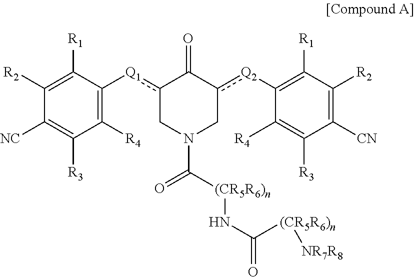

wherein
Q₁ and Q₂ are each independently, either CH or CH₂;
R₁, R₂, R₃ and R₄ are each independently selected from: H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₅ and R₆ are each, independently, selected from: H, F, Cl, Br, I, OH, R₁₅—OH (e.g., CH₂—OH), COOH, CN, C₁-C₁₀ alkyl (e.g., iPr), OR₁₃ (e.g., OMe), NH₂, N(R₁₃)(R₁₄) (e.g., N(CH₃)₂), substituted or unsubstituted (C₃-C₈) cycloalkyl, substituted or unsubstituted (C₃-C₈) heterocyclic ring having one or more heteroatoms selected from N, O and S; or R₅ and R₆ are joint to form a substituted or unsubstituted (C₃-C₈) cycloalkyl (e.g., cyclopropyl) or a substituted or unsubstituted (C₃-C₈) heterocyclic ring (e.g. morpholine); wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₅) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
n is an integer between 1 and 15;
R₇ and R₈ are each, independently, selected from: H, F, Cl, Br, I, substituted or unsubstituted linear or branched C₁-C₁₀ alkyl (e.g. methyl, ethyl, propyl, iso-propyl, butyl, sec-butyl, tert-butyl), substituted or unsubstituted linear or branched C₁-C₁₀ alkoxy, substituted or unsubstituted aryl, substituted or unsubstituted heteroaryl, C(O)—R₁₃, S(O)—R₁₃, S(O)₂—R₁₃, R₁₅-Ph, R₁₅-aryl, R₁₅-heteroaryl, R₁₅-R₁₃, R₁₅-R₁₆-R₁₃ (e.g., CH₂—C≡CH, —CH₂—CH═CH—C₁-C₁₀ alkyl, —CH₂—CH═CH₂, substituted or unsubstituted (C₃—C) cycloalkyl, substituted or unsubstituted (C₃—C) heterocyclic ring having one or more heteroatoms selected from N, O and S; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q1}R₁₃;
R₁₃ and R₁₄ are each independently selected from: H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, halogen, N₃, and CN;
R₁₅ is [CH₂]ₚ
   wherein p is between 1 and 10; and
R₁₆ is [CH]_{q}, [C]_{q};
   wherein q is between 2 and 10;
      or geometrical isomer, optical isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal thereof.

In some embodiments R₁, R₂, R₃, and R₄ are H. In some embodiments R₁, R₂, R₃, and R₄ are each independently H, NO₂, OH, COOH, NH₂, F, Cl, Br, I, CN, R₁₃, OR₁, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, or S(O)₂R₁₃; each is a separate embodiment according to this invention. In some embodiments R₂ is Cl. In some embodiments R₄ and R₂ are C1. In some embodiments R₂ is F. In some embodiments R₂ is Br. In some embodiments R₂ is I. In some embodiments R₂ is CN. In some embodiments R₂ is NO₂. In some embodiments R₂ is CF₃.

In some embodiments Q₁ and Q₂ are both CH. In some embodiments Q₁ is CH and Q₂ is CH₂. In some embodiments Q₁ and Q₂ are both CH₂.

In some embodiments R₅ and R₆ are the same. In some embodiments R₅ and R₆ are each independently F. In some embodiments, R₅ and R₆ are each independently selected from: H, F, Cl, Br, I, OH, R₁₅—OH (e.g., CH₂—OH), COOH, CN, C₁-C₁₀ alkyl (e.g., iPr), OR₁₃ (e.g., OMe), NH₂, N(R₁₃)(R₁₄) (e.g., N(CH₃)₂), substituted or unsubstituted (C₃-C₈) cycloalkyl, substituted or unsubstituted (C₃-C₈) heterocyclic ring having one or more heteroatoms selected from N, O and S; each represents a separated embodiment according to this invention. In some embodiments, the substitutions are at least one of: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃; each represents a separated embodiment according to this invention. In some embodiments R₅ and R₆ are each independently OH. In some embodiments R₅ and R₆ are each independently R₁₅—OH. In some embodiments R₅ and R₆ are each independently CH₂—OH. In some embodiments R₅ and R₆ are each independently COOH. In some embodiments R₅ and R₆ are each independently C₁-C₁₀ alkyl. In some embodiments R₅ and R₆ are both C₁-C₁₀ alkyl. In some embodiments R₅ and R₆ are each independently iPr. In some embodiments, R₅ and R₆ are each independently methyl. In some embodiments R₅ and R₆ are each independently OR₁₃. In some embodiments R₅ and R₆ are each independently OMe. In some embodiments R₅ and R₆ are each independently NH₂. In some embodiments R₅ and R₆ are each independently N(R₁₃)(R₁₄). In some embodiments R₅ and R₆ are each independently N(CH₃)₂. In some embodiments, R₅ and R₆ are joint to form a substituted or unsubstituted (C₃-C₈) cycloalkyl. In some embodiments, R₅ and R₆ are joint to form a cyclopropyl. In some embodiments, R₅ and R₆ are joint to form a substituted or unsubstituted (C₃-C₈) heterocyclic ring. In some embodiments, R₅ and R₆ are joint to form a morpholine ring. In some embodiments R₅ and R₆ are both H. In some embodiments R₅ and R₆ are each independently H. In some embodiments, R₅ is H and R₆ is R₁₅—OH.

In some embodiments, R₇ and R₈ are different. In some embodiments, R₇ and R₈ are the same. In some embodiments R₇ and R₈ are each independently H. In some embodiments, R₇ and R₈ are each independently a substituted or unsubstituted linear or branched C₁-C₁₀ alkyl. In some embodiments, R₇ and R₈ are each independently a methyl. In some embodiments, R₇ and R₈ are both a methyl. In some embodiments, R₇ and R₈ are each independently an ethyl, a propyl, an iso-propyl, a butyl, an iso-butyl, a tert-butyl, a pentyl; each is a separate embodiment according to this invention. In some embodiments, R₇ is an ethyl, a propyl, an iso-propyl, a butyl, an iso-butyl, a tert-butyl, a pentyl and R₈ is a methyl; each is a separate embodiment according to this invention. In some embodiments, R₇ is an ethyl, a propyl, an iso-propyl, a butyl, an iso-butyl, a tert-butyl, a pentyl and R₈ is H; each is a separate embodiment according to this invention. In some embodiments, R₇ and R₈ are each independently a substituted C₁-C₁₀ alkyl. In some embodiments, R₇ and R₈ are each independently an C₁-C₁₀ alkyl substituted with N₃. In some embodiments, R₇ and R₈ are each independently a C₃ alkyl substituted with N₃. In some embodiments, R₇ is a C₃ alkyl substituted with N₃ and R₈ is a methyl. In some embodiments, R₇ and R₈ are each independently a R₁₅-R₁₆-R₁₃. In some embodiments, R₇ is R₁₅-R₁₆-R₁₃, and R₁₅ is CH₂, R₁ is [C]_{q}, q is 2 and R₁₃ is H. In some embodiments, R₇ and R₈ are each independently CH₂—C≡CH. In some embodiments, R₇ is CH₂—C≡CH and R₈ is a methyl. In some embodiments, R₇ and R₈ are each independently a substituted or unsubstituted aryl. In some embodiments, R₇ and R₈ are each independently a substituted or unsubstituted heteroaryl. In some embodiments, R₇ and R₈ are each independently substituted with at least one selected from: C₁-C₁₄ linear or branched haloalkyl, C₁-C₁₄ linear or branched alkoxy, C₁-C₁₄ linear or branched alkenyl, C₁-C₁₄ linear or branched alkynyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q}1R₁₃; each is a separate embodiment according to this invention. In some embodiments, R₇ and R₈ are each independently C(O)—CH₃. In some embodiments, R₇ and R₈ are each independently S(O)₂—CH₃. In some embodiments, R₇ and R₈ are each independently R₁₅-aryl.

In some embodiments, R₁₃ and R₁₄ are different. In some embodiments, R₁₃ and R₁₄ are the same. In some embodiments, R₁₃ and R₁₄ are each independently H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, halogen, N₃, and CN; each is a separate embodiment according to this invention. In some embodiments, R₁₃ and R₁₄ are each independently H. In some embodiments, R₁₃ and R₁₄ are each independently a methyl. In some embodiments, R₁₃ and R₁₄ are each independently methoxyethyl. In some embodiments, R₁₃ and R₁₄ are each independently substituted or unsubstituted aryl. In some embodiments, R₁₃ and R₁₄ are each independently phenyl. In some embodiments, R₁₃ and R₁₄ are each independently substituted or unsubstituted heteroaryl. In some embodiments, R₁₃ and R₁₄ are each independently pyridyl. In some embodiments, R₁₃ and R₁₄ are each independently C(O)—CH₃. In some embodiments, R₁₃ is H. In some embodiments, R₁₃ and R₁₄ are each independently —C(O)—C1-C₁₄ substituted or unsubstituted linear or branched alkyl, In some embodiments, R₁₃ and R₁₄ are each independently —C(O)—CH₃. In some embodiments, R₁₃ and R₁₄ are each independently OH. In some embodiments, R₁₃ and R₁₄ are each independently a substituted or unsubstituted C₁-C₁₄ linear or branched alkyl group. In some embodiments, R₁₃ and R₁₄ are each independently a substituted C₁-C₁₄ linear or branched alkyl group, substituted with N₃. In some embodiments, R₁₃ and R₁₄ are each independently a substituted C₁-C₁₄ linear or branched alkyl group, substituted with C₁-C₁₄ linear or branched alkynyl. In some embodiments, R₁₃ and R₁₄ are each independently substituted with C₁-C₁₄ linear or branched alkoxy. In some embodiments, R₁₃ and R₁₄ are each independently substituted with C₁-C₁₄ linear or branched methoxy. In some embodiments, R₁₃ is methyl. In some embodiments, R₁₃ and R₁₄ are each independently C(O)—C₁-C₁₄ linear or branched alkyl. In some embodiments, R₁₃ and R₁₄ are each independently C₁-C₁₄ linear or branched-S(O)₂-alkyl. In some embodiments, R₁₃ and R₁₄ are each independently Cl. In some embodiments, R₁₃ and R₁₄ are each independently Br. In some embodiments, R₁₃ and R₁₄ are each independently I. In some embodiments, R₁₃ and R₁₄ are each independently F.

In some embodiments, R₁₅ is CH₂. In some embodiments, R₁₅ is [CH₂]₂. In some embodiments, R₁₅ is [CH₂]₃. In some embodiments, R₁₅ is [CH₂]₄.

In some embodiment, p is 1. In some embodiment, p is 2. In some embodiment, p is 3. In some embodiment, p is 4. In some embodiment, p is 5. In some embodiment, p is 6. In some embodiment, p is 7.

In some embodiments, R₁₆ is [CH]_{q}. In some embodiments, R₁₆ is [C]_{q}.

In some embodiments, q is 2. In some embodiments, q is 3. In some embodiments, q is 4. In some embodiments, q is 5. In some embodiments, q is 6.

In some embodiment, n is 1. In some embodiment, n is 2. In some embodiment, n is 3. In some embodiment, n is 4. In some embodiment, n is 5. In some embodiment, n is 6. In some embodiment, n is 7.

In some embodiments, R₇ is R₁₅-R₁₆-R₁₃, and R₁₅ is CH₂, R₁ is [C]_{q}, q is 2 and R₁₃ is H.

In some embodiments, Compound A is represented by the structure of Compound B1, B2, B3 and C1 as described herein below; or a geometrical isomer, optical isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal thereof; each represents a separate embodiment according to this invention.

In some embodiments, the present invention relates to a compound, represented by the structure of formula IV:

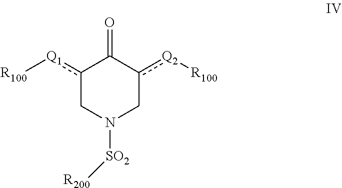

wherein
Q₁ and Q₂ are each independently, either CH or CH₂,
R₁₀₀ is selected from:
   (i) phenyl, optionally substituted with 1-5 substituents (i.e., aryl) selected from the group consisting of: F, Cl, Br, I, OH, R₁₃, OR₁, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl;
   (ii) naphthyl, optionally substituted with 1-5 substituents selected from the consisting of F, Cl, Br, I, OH, R₁₃, OR₁, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl;
   (iii) a 5 or 6 membered monocyclic heteroaryl group, having 1-3 heteroatoms selected from the group consisting of 0, N, and S, optionally substituted with 1-3 substituents selected from the group consisting of: F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl;
   (iv) an 8 to 10 membered bicyclic heteroaryl group containing 1-3 heteroatoms selected from the group consisting of O, N, and S; and the second ring is fused to the first ring using 3 to 4 carbon atoms, and the bicyclic heteroaryl group is optionally substituted with 1-3 substituents selected from the group consisting of F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl; and
   (v) a substituted or unsubstituted C₁-C₅ linear or branched alkyl or a substituted or unsubstituted C₁-C₅ linear or branched alkene wherein substitutions include at least one selected of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, aryl, phenyl, heteroaryl, OH, COOH, NH₂, N(R₁₃)(R₁₄), N₃, CF₃, CN or NO₂;
R₂₀₀ is amine (—NR₁₃R₁₄), OH, —OCOR₁₃, OR₁₃, substituted or unsubstituted linear or branched (C₁-C₁₄) alkyl, substituted or unsubstituted linear or branched (C₁-C₁₄) alkyl-NR₁₃R₁₄, substituted or unsubstituted linear or branched (C₁-C₁₄) alkyl-NHR₁₃, substituted or unsubstituted linear or branched (C₂-C₁₄) alkenyl-NR₁₃R₁₄, substituted or unsubstituted linear or branched (C₂-C₁₄) alkenyl-NHR₁₃, substituted or unsubstituted linear or branched (C₁-C₁₄) alkyl-OR₁₃, substituted or unsubstituted (C₃-C₈) cycloalkyl, substituted or unsubstituted (C₃-C₈) heterocyclic ring, R₁₅—N(R₁₃)(R₁₄), R₁₅—O(R₁₃), R₁₅—Cl, R₁₅—Br, R₁₅—F, R₁₅—I, R₁₅—N₃, R₁₅—CH═CH₂, and R₁₅—C≡CH; wherein substitutions include at least one selected of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, OH, COOH, NH₂, N(R₁₃)(R₁₄), N₃, CF₃, CN or NO₂;
R₁₃ and R₁₄ are each independently selected from: H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; —substituted or unsubstituted aryl (e.g., phenyl), substituted or unsubstituted heteroaryl (e.g., pyridyl), —C(O)—C₁-C₁₄ substituted or unsubstituted linear or branched alkyl (e.g., C(O)—CH₃), or —S(O)₂—C1-C₁₄ substituted or unsubstituted linear or branched alkyl, wherein substitutions are selected from F, Cl, Br, I, C₁-C₁₄ linear or branched alkyl, C₁-C₁₄ linear or branched haloalkyl, C₁-C₁₄ linear or branched alkoxy, C₁-C₁₄ linear or branched alkenyl, C₁-C₁₄ linear or branched alkynyl (e.g. CH₂—C≡CH), aryl, phenyl, heteroaryl, NO₂, OH, COOH, NH₂, C₁-C₁₄ alkylamino, C₁-C₁₄ dialkylamino, N₃, and CN;
R₁₅ is [CH₂]ₚ
   wherein p is between 1 and 10; and
R₁₆ is [CH]_{q}, [C]_{q}
   wherein q is between 2 and 10;
      or geometrical isomer, optical isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal thereof.

In some embodiments, compound of Formula IV is represented by the structure of Compound AA, BA, CA, D1, E1, F1, A2, C2, C3, BA-2, CA-2, F1-5, E1-2 or AA-8 as described herein below; or a geometrical isomer, optical isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal thereof; each represents a separate embodiment according to this invention.

The compounds of Formula IV include both unreduced and reduced species. For example, without limitation, in some embodiments, the compound of Formula IV is in an unreduced form, i.e., where both of Q₁ and Q₂ are CH, and has the following structure:

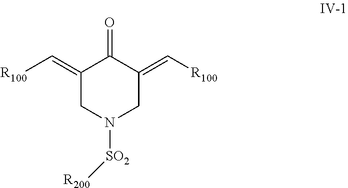

In other embodiments, the compound of Formula IV is in a partially reduced form, i.e., where one of Q₁ or Q₂ is CH₂ and the other is CH, and has the following structure:

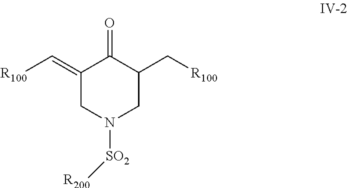

In some embodiments, the compound of Formula IV is in a reduced form, i.e., wherein both of Q₁ and Q₂ are CH₂.

In some embodiments, the present invention relates to a compound represented by the structure of Formula IV-1:

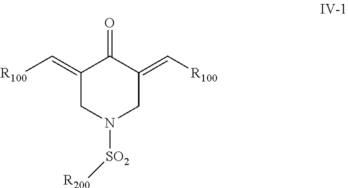

wherein
R₁₀₀ is a phenyl, optionally substituted with 1-5 substituents (i.e., aryl) selected from the group consisting of F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl; and
R₂₀₀ is amine (—NR₁₃R₁₄), OH, —OCOR₁₃, OR₁₃, substituted or unsubstituted linear or branched (C₁-C₁₄) alkyl, substituted or unsubstituted linear or branched (C₁-C₁₄) alkyl-NR₁₃R₁₄, substituted or unsubstituted linear or branched (C₁-C₁₄) alkyl-NHR₁₃, substituted or unsubstituted linear or branched (C₂-C₁₄) alkenyl-NR₁₃R₁₄, substituted or unsubstituted linear or branched (C₂-C₁₄) alkenyl-NHR₁₃, substituted or unsubstituted linear or branched (C₁-C₁₄) alkyl-OR₁₃, substituted or unsubstituted (C₃-C₈) cycloalkyl, substituted or unsubstituted (C₃-C₈) heterocyclic ring, R₁₅—N(R₁₃)(R₁₄), R₁₅—O(R₁₃), R₁₅—C1, R₁₅—Br, R₁₅—F, R₁₅—I, R₁₅—N₃, R₁₅—CH═CH₂, and R₁₅—C≡CH; wherein substitutions include at least one selected of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, OH, COOH, NH₂, N(R₁₃)(R₁₄), N₃, CF₃, CN or NO₂;
R₁₃ and R₁₄ are each independently selected from: H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl (e.g. CH₂—C≡CH), aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₅ is [CH₂]ₚ
   wherein p is between 1 and 10; and
R₁₆ is [CH]_{q}, [C]_{q}
   wherein q is between 2 and 10;
      or geometrical isomer, optical isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal thereof.

In some embodiments, Q₁ and Q₂ of compound of formula IV are both CH. In some embodiments, Q₁ CH and Q₂ is CH₂. In some embodiments, Q₁ and Q₂ are both CH₂.

In some embodiments, R₁₀₀ of compound of formula IV or IV-1 is an aryl represented by the structure of formula V:

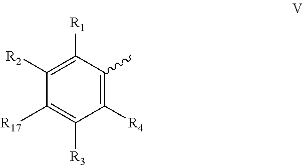

wherein
R₁, R₂, R₃, R₄ and R₁₇ of compound of formula V are each independently selected from:
   H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —N(R₁₃)(R₁₄), —CON(R₁₃)(R₁₄), N₃, S(O)R₁₃, and S(O)₂R₁₃;
R₁₃ and R₁₄ are each independently selected from: H, Cl, Br, I, F, OH, substituted or unsubstituted C₁-C₁₄ linear or branched alkyl group (e.g., methyl, methoxyethyl), substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), OH, —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl (e.g. CH₂—C≡CH), aryl, phenyl, heteroaryl, NO₂, OH, COOH, NH₂, C₁-C₁₄ alkylamino, C₁-C₁₄ dialkylamino, halogen, N₃, and CN;
R₁₅ is [CH₂]ₚ
   wherein p is between 1 and 10; and
R₁₆ is [CH]_{q}, [C]_{q}
   wherein q is between 2 and 10;

In some embodiments R₁₇, R₁, R₂, R₃, and R₄ of Formula V are each independently H. In some embodiments R₁₇, R₁, R₂, R₃, and R₄ are each independently Cl. In some embodiments R₁₇, R₁, R₂, R₃, and R₄ are each independently Br. In some embodiments R₁₇, R₁, R₂, R₃, and R₄ are each independently F. In some embodiments R₁₇, R₁, R₂, R₃, and R₄ are each independently I. In some embodiments R₁₇, R₁, R₂, R₃, and R₄ are each independently CN. In some embodiments R₁₇, R₁, R₂, R₃, and R₄ are each independently NO₂. In some embodiments R₁₇, R₁, R₂, R₃, and R₄ are each independently CF₃. In some embodiments R₂ is Cl. In some embodiments R₂ is F. In some embodiments R₂ is Br. In some embodiments R₂ is I. In some embodiments R₂ is CN. In some embodiments R₂ is NO₂. In some embodiments R₂ is CF₃. In some embodiments R₁₇ is Cl. In some embodiments R₁₇ is F. In some embodiments R₁₇ is Br. In some embodiments R₁₇ is I. In some embodiments R₁₇ is CN. In some embodiments R₁₇ is NO₂.

In some embodiments, R₁₀₀ of compound of formula IV, IV-1, or IV-2 is a substituted 5 or 6 membered monocyclic heteroaryl group, having 1-3 heteroatoms selected from the group consisting of O, N, and S. In some embodiments, R₁₀₀ is a substituted or unsubstituted furan, pyrrole, oxazole, isoxazole, oxadiazole, 2-, 3- or 4-pyridine, pyrazine, pyrimidine, pyridazine, triazine, thiophene, thiazole, isothiazole, thiadiazole, imidazole, indazole, diazole, triazole, tetrazole; each is a separate embodiment according to this invention. In some embodiments, R₁₀₀ is a substituted isoxazole. In some embodiments, R₁₀₀ is a dimethyl substituted isoxazole. In some embodiments, R₁₀₀ is a heteroaryl represented by the structure of formula VI:

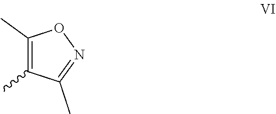

In some embodiments, R₁₀₀ of compound of formula IV, IV-1, or IV-2 is a phenyl. In some embodiments, R₁₀₀ is a substituted phenyl, i.e., aryl. In some embodiments, R₁₀₀ is an aryl. In some embodiments, R₁₀₀ is an aryl substituted with at least one selected from: F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, wherein substitutions include at least one selected of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, aryl, phenyl, heteroaryl, OH, COOH, NH₂, N(R₁₃)(R₁₄), N₃, CF₃, CN or NO₂; each is a separate embodiment according to this invention. In some embodiments, R₁₀₀ is an aryl substituted with at least one selected of: F, Cl, Br, I, CF₃, CN, NO₂ or any combination thereof. In some embodiments, R₁₀₀ is an aryl substituted with at least one selected of: F, Cl, CF₃, CN, NO₂ or any combination thereof.

In some embodiments, R₁₀₀ of compound of formula IV, IV-1, or IV-2 is a naphthyl. In some embodiments, R₁₀₀ is a substituted naphthyl, substituted with 1-5 substituents selected from: F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, wherein substitutions include at least one selected of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, aryl, phenyl, heteroaryl, OH, COOH, NH₂, N(R₁₃)(R₁₄), N₃, CF₃, CN or NO₂; each substitution is a separate embodiment according to this invention. In some embodiments, R₁₀₀ is a substituted naphthyl, substituted with 1-5 substituents selected from: F, Cl, Br, I, CF₃, OCF₃, CN or NO₂.

In some embodiments, R₁₀₀ of compound of formula IV, IV-1, or IV-2 is a 5 or 6 membered monocyclic heteroaryl group, having 1-3 heteroatoms selected from the group consisting of 0, N, and S. In some embodiments, R₁₀₀ is a 5 or 6 membered monocyclic heteroaryl substituted with 1-3 substituents selected from the group consisting of: F, Cl, Br, I, OH, C₁-C₁₄ linear or branched alkyl (e.g. methyl), C₁-C₁₄ linear, branched or cyclic alkoxy, CF₃, CN or NO₂; each substitution is a separate embodiment according to this invention. In some embodiments, R₁₀₀ is a substituted or unsubstituted isoxazole. In some embodiments, R₁₀₀ is a substituted or unsubstituted furan, pyrrole, oxazole, isoxazole, oxadiazole, 2-, 3- or 4-pyridine, pyrazine, pyrimidine, pyridazine, triazine, thiophene, thiazole, isothiazole, thiadiazole, imidazole, indazole, diazole, triazole, tetrazole; each is a separate embodiment according to this invention. In some embodiments, R₁₀₀ is substituted with at least one selected from: F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C1-C₅ linear or branched haloalkoxy, C₁-C₅ linear or branched alkoxyalkyl, wherein substitutions include at least one selected of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, aryl, phenyl, heteroaryl, OH, COOH, NH₂, N(R₁₃)(R₁₄), N₃, CF₃, CN or NO₂; each substitution is a separate embodiment according to this invention. In some embodiments, R₁₀₀ is substituted with C₁-C₁₄ linear, branched or cyclic alkyl. In some embodiments, R₁₀₀ is substituted with at least one methyl. In some embodiments, R₁₀₀ is substituted with two methyls.

In some embodiments, R₁₀₀ of compound of formula IV, IV-1, or IV-2 is an 8 to 10 membered bicyclic heteroaryl group. In some embodiments, R₁₀₀ is a 8 to 10 membered bicyclic heteroaryl group wherein the second ring is fused to the first ring using 3 to 4 carbon atoms. In some embodiments, R₁₀₀ is substituted with F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, wherein substitutions include at least one selected of: F, Cl, Br, I, C₁-C₅ linear or branched alkyl, C₁-C₁₄ lin R₁₅—Cl, R₁₅—Br, R₁₅—F, R₁₅—I ear or branched haloalkyl, C₁-C₁₄ linear or branched alkoxy, C₁-C₁₄ linear or branched alkenyl, aryl, phenyl, heteroaryl, OH, COOH, NH₂, N(R₁₃)(R₁₄), N₃, CF₃, CN or NO₂; each is a separate embodiment according to this invention.

In some embodiments, R₁₀₀ of compound of formula IV, IV-1, or IV-2 is a substituted or unsubstituted C₁-C₅ linear or branched alkyl. In some embodiments, R₁₀₀ is a substituted or unsubstituted C₁-C₅ linear or branched alkene. In some embodiments, R₁₀₀ is substituted with at least one of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, aryl, phenyl, heteroaryl, OH, COOH, NH₂, N(R₁₃)(R₁₄), N₃, CF₃, CN or NO₂; each is a separate embodiment according to this invention.

In some embodiments, R₂₀₀ of compound of formula IV, IV-1, or IV-2 is an amine (—NR₁₃R₁₄). In some embodiments, R₂₀₀ is OH. In some embodiments, R₂₀₀ is —OCOR₁₃. In some embodiments, R₂₀₀ is OR₁₃. In some embodiments, R₂₀₀ is substituted or unsubstituted linear or branched (C₁-C₁₄) alkyl. In some embodiments, R₂₀₀ is substituted or unsubstituted linear or branched (C₁-C₁₄) alkyl-NR₁₃R₁₄. In some embodiments, R₂₀₀ is a dimethyl-propylamine. In some embodiments, R₂₀₀ is a dimethyl-ethylamine. In some embodiments, R₂₀₀ is substituted or unsubstituted linear or branched (C₁-C₁₄) alkyl-NHR₁₃. In some embodiments, R₂₀₀ is substituted or unsubstituted linear or branched (C₂-C₁₄) alkenyl-NR₁₃R₁₄. In some embodiments, R₂₀₀ is substituted or unsubstituted linear or branched (C₂-C₁₄) alkenyl-NHR₁₃. In some embodiments, R₂₀₀ is substituted or unsubstituted linear or branched (C₁-C₁₄) alkyl-OR₁₃. In some embodiments, R₂₀₀ is substituted or unsubstituted (C₃-C₈) cycloalkyl. In some embodiments, R₂₀₀ is substituted or unsubstituted (C₃-C₈) heterocyclic ring. In some embodiments, R₂₀₀ is R₁₅—N(R₁₃)(R₁₄). In some embodiments, R₂₀₀ is [CH₂]ₚ—N(R₁₃)(R₁₄), wherein p is 2, 3, 4, 5, or 6; each is a separate embodiment according to this invention. In some embodiments, R₂₀₀ is [CH₂]p-N(R₁₃)(R₁₄), wherein R₁₃ and R₁₄ are each independently H, methyl, ethyl, propyl, i-propyl, butyl, t-butyl or pentyl; each is a separate embodiment according to this invention. In some embodiments, R₂₀₀ is [CH₂]p-N(R₁₃)(R₁₄), wherein R₁₃ and R₁₄ are both methyls. In some embodiments, R₂₀₀ is [CH₂]p-N(R₁₃)(R₁₄), wherein R₁₃ is methyl and R₁₄ is a substituted C₁-C₁₄ linear or branched alkyl group. In some embodiments, R₂₀₀ is [CH₂]p-N(R₁₃)(R₁₄), wherein R₁₄ is a substituted C₁-C₁₄ linear or branched alkyl group, substituted with N₃, C₁-C₁₄ linear or branched alkenyl, or C₁-C₁₄ linear or branched alkynyl; each is a separate embodiment according to this invention. In some embodiments, R₂₀₀ is R₁₅—O(R₁₃). In some embodiments, R₂₀₀ is [CH₂]p-OR₁₃ wherein R₁₃ is H, methyl, ethyl, propyl, i-propyl, butyl, t-butyl or pentyl; each is a separate embodiment according to this invention. In some embodiments, R₂₀₀ is [CH₂]p-OCH₃. In some embodiments, R₂₀₀ is R₁₅—N₃. In some embodiments, R₂₀₀ is R₁₅—CH═CH₂. In some embodiments, R₂₀₀ is R₁₅—C≡CH. In some embodiments, R₂₀₀ is R₁₅—Cl. In some embodiments, R₂₀₀ is R₁₅—Br. In some embodiments, R₂₀₀ is R₁₅—F. In some embodiments, R₂₀₀ is R₁₅—I. In some embodiments, R₂₀₀ is substituted with at least one of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, OH, COOH, NH₂, N(R₁₃)(R₁₄), N₃, CF₃, CN and NO₂; each is a separate embodiment according to this invention.

In some embodiments, R₁₃ and R₁₄ of compound of formula IV, IV-1, or IV-2 are the same. In some embodiments, R₁₃ and R₁₄ are different. In some embodiments, R₁₃ and R₁₄ are each independently methyl. In some embodiments, R₁₃ and R₁₄ are both methyl. In some embodiments, R₁₃ and R₁₄ are each independently substituted or unsubstituted linear or branched (C₁-C₁₄) alkyl. In some embodiments, R₁₃ and R₁₄ are each independently substituted linear or branched (C₁-C₁₄) alkyl, wherein the alkyl is substituted with: F, Cl, Br, I, C₁-C₁₄ linear or branched alkyl, C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, N₃, or CN. In some embodiments, R₁₃ and R₁₄ are each independently a substituted linear (C₁-C₈) alkyl, wherein the alkyl is substituted with: C₁-C₁₄ linear or branched alkenyl, C₁-C₁₄ linear or branched alkynyl, or N₃. In some embodiments, R₁₃ and R₁₄ are each independently ethyl, propyl, iso-propyl, butyl, iso-butyl, tert-butyl, sec-butyl, pentyl, iso-pentyl, neo-pentyl, hexyl, or heptyl; each represents a separate embodiment according to this invention. In some embodiments, R₁₃ and R₁₄ are each independently a (C₁-C₁₄) alkyl substituted with alkenyl, alkynyl, or azide; each represents a separate embodiment according to this invention. In some embodiments, R₁₃ and R₁₄ are each independently a (C₃-C₈) cycloalkyl. In some embodiments, R₁₃ and R₁₄ are each independently a (C₃-C₈) heterocyclic ring. In some embodiments, R₁₃ and R₁₄ are each independently Cl. In some embodiments, R₁₃ and R₁₄ are each independently Br. In some embodiments, R₁₃ and R₁₄ are each independently I. In some embodiments, R₁₃ and R₁₄ are each independently F.

In some embodiments, pharmaceutically acceptable salts of compound of formula I, II, III, IV, IV-1 or Compound A include, without limitation, phosphate, methane sulfonate, hydrochloride, sulphate, citrate, and p-toluene sulfonate salts.

As used herein, the term “geometric isomers” refers to “cis-trans isomers”, “E-Z isomers”, or to “configurational isomers”. Geometric isomers are stereoisomers, that is, pairs of molecules which have the same formula but whose functional groups are rotated into a different orientation in three-dimensional space. In general, geometric isomers contain double bonds that do not rotate, or they may contain ring structures, where the rotation of bonds is restricted or prevented. In some embodiments, geometric isomers refer to cis-trans isomers. In other embodiments, geometric isomers refer to E-Z isomers.

For example, without limitation, the following Compounds A-C1 and A-C2, as well as pharmaceutically acceptable salts thereof, are geometric isomers of Compound A, wherein Q₁ is CH, and are included as suitable embodiments of Compound A in accordance with the present invention as described herein:

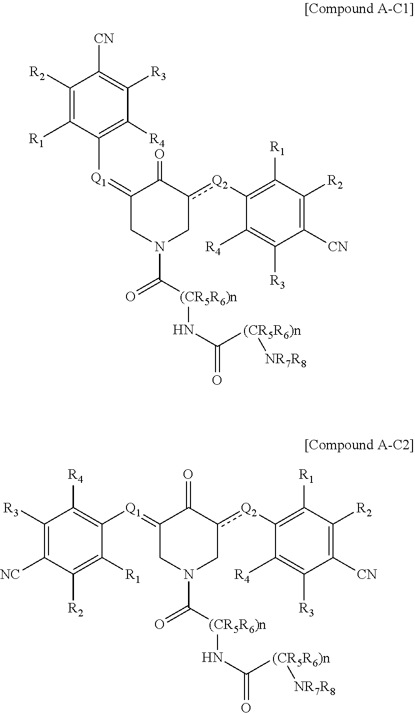

Compound A and/or Compound of formula I—IV include both unreduced and reduced species. For example, in some embodiments, Compound A, is in an unreduced form, i.e., where both of Q₁ and Q₂ are CH, and R₁ to R₈ are each as recited hereinabove, and has the following structure:

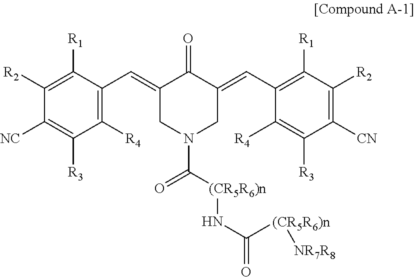

In other embodiments, the compound is in a partially reduced form, i.e., wherein either one of Q₁ or Q₂ is CH₂ and the other is CH, and R₁ to R₈ are each as recited hereinabove, and has the following structure:

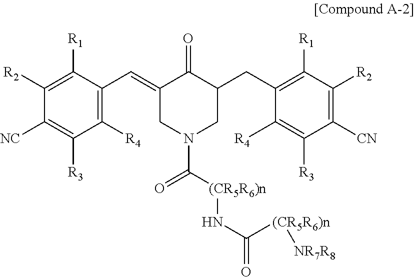

In some embodiments, the compound is in a reduced form wherein both of Q₁ and Q₂ are CH₂.

Compound A and compounds of formula I-IV, IV-1 may also include optical isomers of such unreduced, partially reduced or reduced compounds.

In some embodiments, the compounds according to this invention are listed in Table A below:

TABLE A

| Compound name | Structure |
|---|---|
| AA | 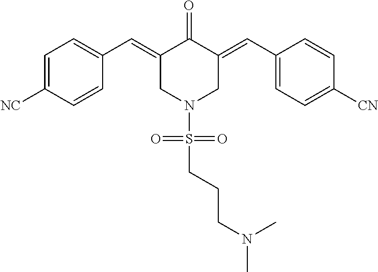 [SMILES: CN(C)CCCS(=O)(=O)N1C/C(=C\c2ccc(C#N)cc2)C(=O)/C(=C/c2ccc(C#N)cc2)C1] |
| A2 | 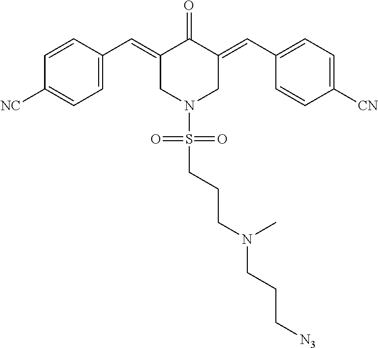 [SMILES: CN(CCCN=[N+]=[N-])CCCS(=O)(=O)N1C/C(=C\c2ccc(C#N)cc2)C(=O)/C(=C/c2ccc(C#N)cc2)C1] |
| A3 | 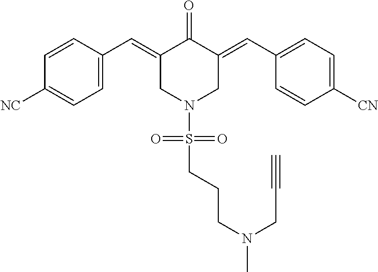 [SMILES: C#CCN(C)CCCS(=O)(=O)N1C/C(=C\c2ccc(C#N)cc2)C(=O)/C(=C/c2ccc(C#N)cc2)C1] |
| BA | 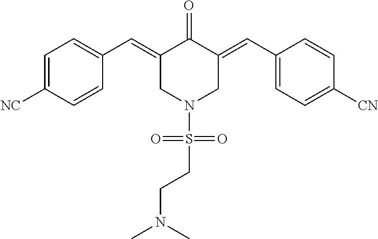 [SMILES: CN(C)CCS(=O)(=O)N1C/C(=C\c2ccc(C#N)cc2)C(=O)/C(=C/c2ccc(C#N)cc2)C1] |
| CA | 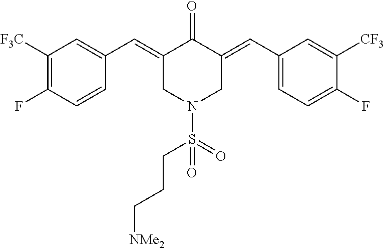 [SMILES: CN(C)CCCS(=O)(=O)N1C/C(=C\c2ccc(F)c(C(F)(F)F)c2)C(=O)/C(=C/c2ccc(F)c(C(F)(F)F)c2)C1] |
| B1 | 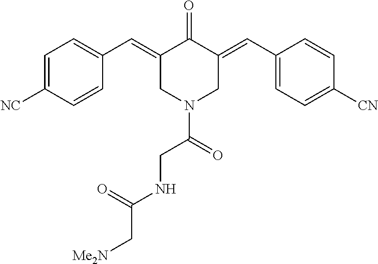 [SMILES: CN(C)CC(=O)NCC(=O)N1C/C(=C\c2ccc(C#N)cc2)C(=O)/C(=C/c2ccc(C#N)cc2)C1] |
| B2 | 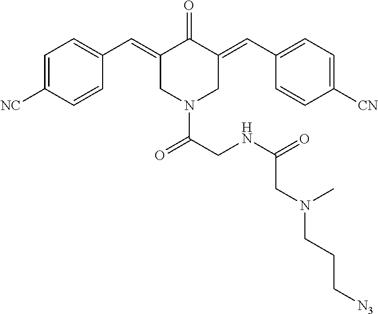 [SMILES: CN(CCCN=[N+]=[N-])CC(=O)NCC(=O)N1C/C(=C\c2ccc(C#N)cc2)C(=O)/C(=C/c2ccc(C#N)cc2)C1] |
| B3 | 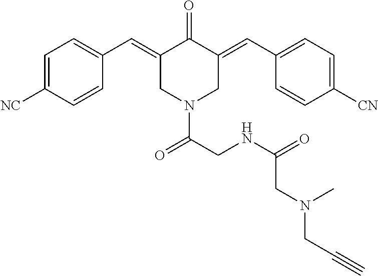 [SMILES: C#CCN(C)CC(=O)NCC(=O)N1C/C(=C\c2ccc(C#N)cc2)C(=O)/C(=C/c2ccc(C#N)cc2)C1] |
| B4 | 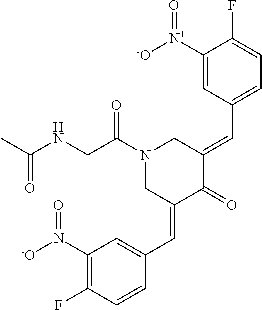 |
| B5 | 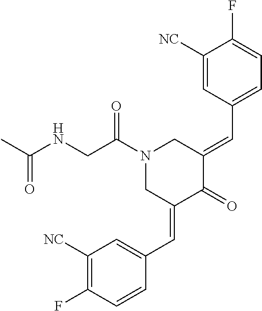 [SMILES: CC(=O)NCC(=O)N1C/C(=C\c2ccc(F)c(C#N)c2)C(=O)/C(=C/c2ccc(F)c(C#N)c2)C1] |
| B6 | 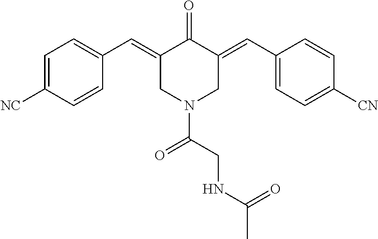 [SMILES: CC(=O)NCC(=O)N1C/C(=C\c2ccc(C#N)cc2)C(=O)/C(=C/c2ccc(C#N)cc2)C1] |
| B7 | 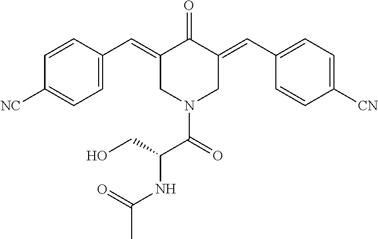 [SMILES: CC(=O)N[C@H](CO)C(=O)N1C/C(=C\c2ccc(C#N)cc2)C(=O)/C(=C/c2ccc(C#N)cc2)C1] |
| B8 | 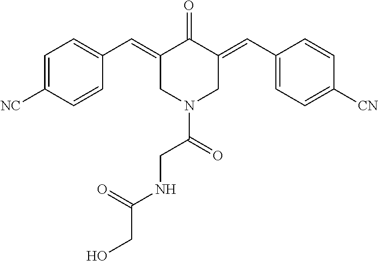 [SMILES: N#Cc1ccc(/C=C2\CN(C(=O)CNC(=O)CO)C/C(=C\c3ccc(C#N)cc3)C2=O)cc1] |
| B9 | 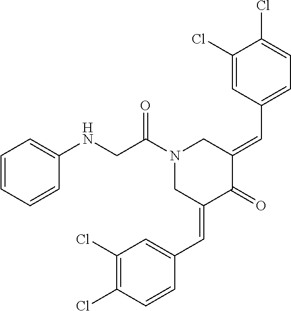 [SMILES: O=C1/C(=C/c2ccc(Cl)c(Cl)c2)CN(C(=O)CNc2ccccc2)C/C1=C\c1ccc(Cl)c(Cl)c1] |
| B10 | 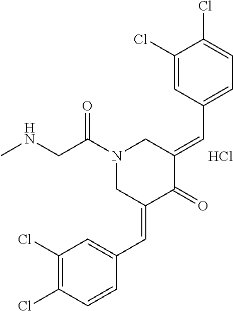 [SMILES: CNCC(=O)N1C/C(=C\c2ccc(Cl)c(Cl)c2)C(=O)/C(=C/c2ccc(Cl)c(Cl)c2)C1 ; Cl] |
| B11 | 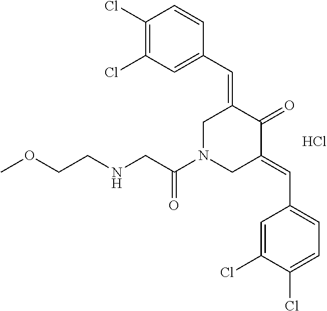 [SMILES: COCCNCC(=O)N1C/C(=C\c2ccc(Cl)c(Cl)c2)C(=O)/C(=C/c2ccc(Cl)c(Cl)c2)C1 ; Cl] |
| B12 | 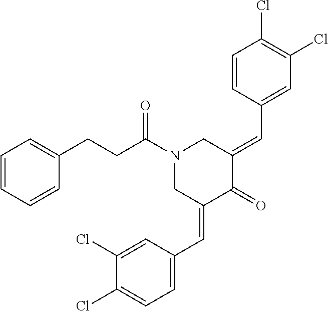 [SMILES: O=C1/C(=C/c2ccc(Cl)c(Cl)c2)CN(C(=O)CCc2ccccc2)C/C1=C\c1ccc(Cl)c(Cl)c1] |
| B13 | 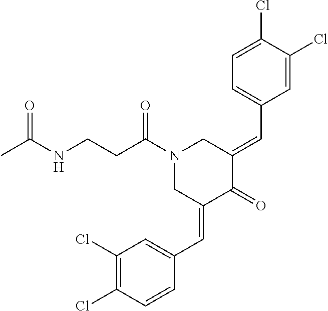 [SMILES: CC(=O)NCCC(=O)N1C/C(=C\c2ccc(Cl)c(Cl)c2)C(=O)/C(=C/c2ccc(Cl)c(Cl)c2)C1] |
| B14 | 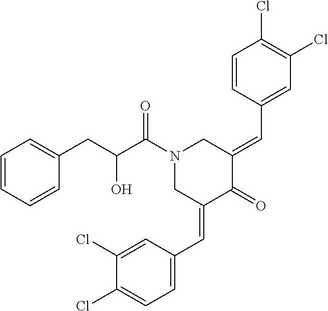 [SMILES: O=C1/C(=C/c2ccc(Cl)c(Cl)c2)CN(C(=O)C(O)Cc2ccccc2)C/C1=C\c1ccc(Cl)c(Cl)c1] |
| B15 | 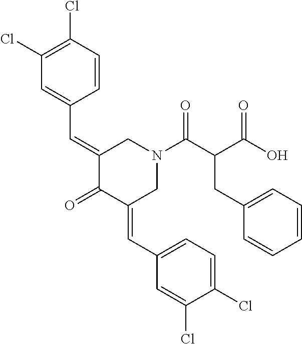 [SMILES: O=C1/C(=C/c2ccc(Cl)c(Cl)c2)CN(C(=O)C(Cc2ccccc2)C(=O)O)C/C1=C\c1ccc(Cl)c(Cl)c1] |
| B16 | 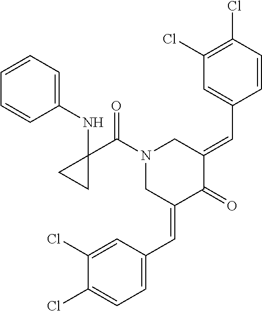 [SMILES: O=C1/C(=C/c2ccc(Cl)c(Cl)c2)CN(C(=O)C2(Nc3ccccc3)CC2)C/C1=C\c1ccc(Cl)c(Cl)c1] |
| B17 | 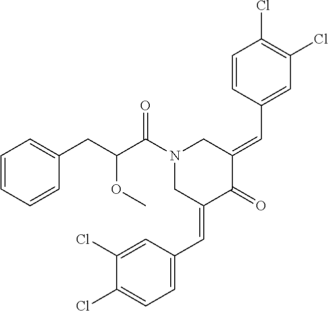 [SMILES: COC(Cc1ccccc1)C(=O)N1C/C(=C\c2ccc(Cl)c(Cl)c2)C(=O)/C(=C/c2ccc(Cl)c(Cl)c2)C1] |
| B18 | 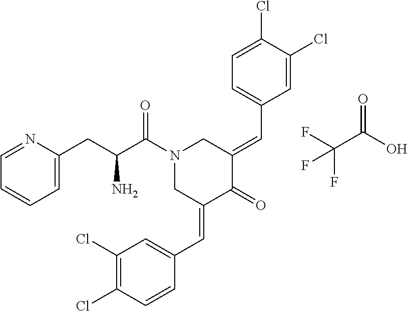 |
| B19 | 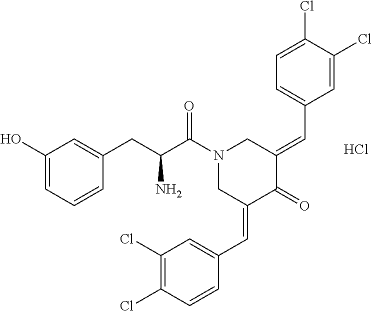 [SMILES: N[C@@H](Cc1cccc(O)c1)C(=O)N1C/C(=C\c2ccc(Cl)c(Cl)c2)C(=O)/C(=C/c2ccc(Cl)c(Cl)c2)C1 ; Cl] |
| B20 | 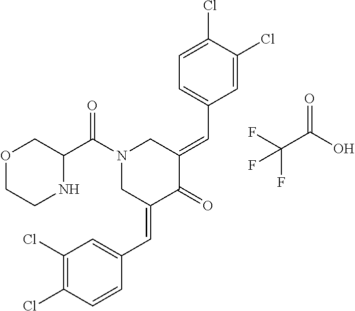 |
| B21 | 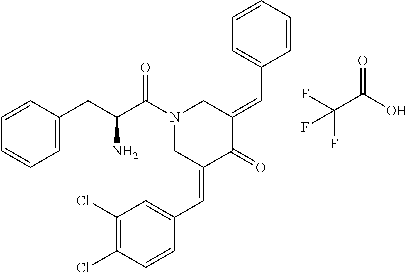 |
| B22 | 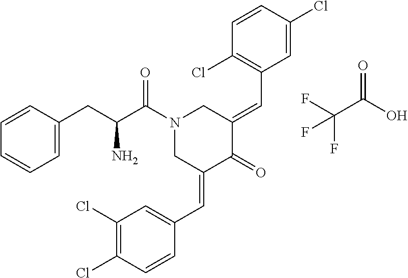 |
| B23 | 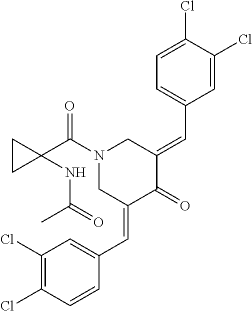 [SMILES: CC(=O)NC1(C(=O)N2C/C(=C\c3ccc(Cl)c(Cl)c3)C(=O)/C(=C/c3ccc(Cl)c(Cl)c3)C2)CC1] |
| B24 | 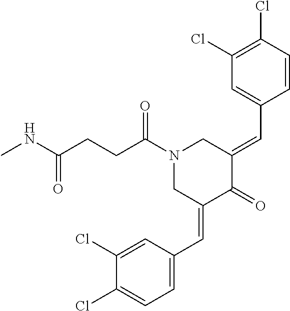 [SMILES: CNC(=O)CCC(=O)N1C/C(=C\c2ccc(Cl)c(Cl)c2)C(=O)/C(=C/c2ccc(Cl)c(Cl)c2)C1] |
| B25 | 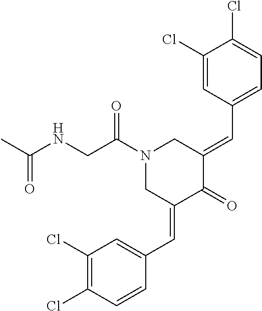 [SMILES: CC(=O)NCC(=O)N1C/C(=C\c2ccc(Cl)c(Cl)c2)C(=O)/C(=C/c2ccc(Cl)c(Cl)c2)C1] |
| B26 | 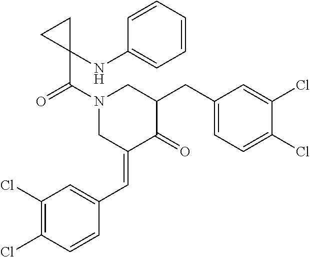 [SMILES: O=C1/C(=C/c2ccc(Cl)c(Cl)c2)CN(C(=O)C2(Nc3ccccc3)CC2)CC1Cc1ccc(Cl)c(Cl)c1] |
| B27 | 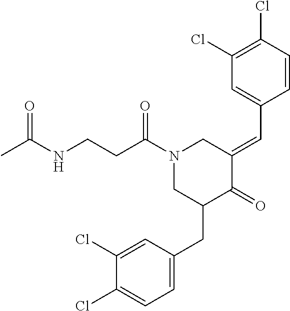 [SMILES: CC(=O)NCCC(=O)N1C/C(=C\c2ccc(Cl)c(Cl)c2)C(=O)C(Cc2ccc(Cl)c(Cl)c2)C1] |
| B28 | 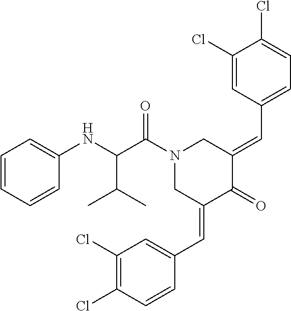 [SMILES: CC(C)C(Nc1ccccc1)C(=O)N1C/C(=C\c2ccc(Cl)c(Cl)c2)C(=O)/C(=C/c2ccc(Cl)c(Cl)c2)C1] |
| B29 | 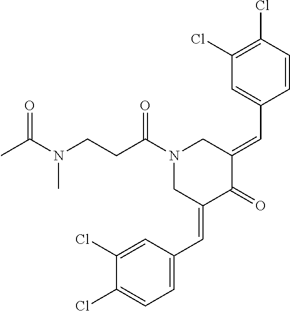 [SMILES: CC(=O)N(C)CCC(=O)N1C/C(=C\c2ccc(Cl)c(Cl)c2)C(=O)/C(=C/c2ccc(Cl)c(Cl)c2)C1] |
| B30 | 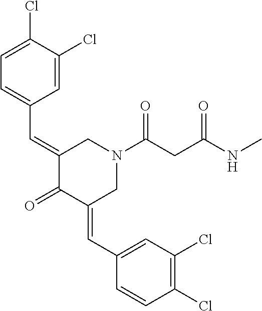 [SMILES: CNC(=O)CC(=O)N1C/C(=C\c2ccc(Cl)c(Cl)c2)C(=O)/C(=C/c2ccc(Cl)c(Cl)c2)C1] |
| B31 | 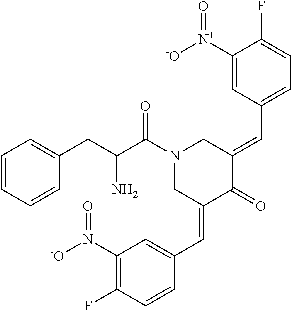 |
| B32 | 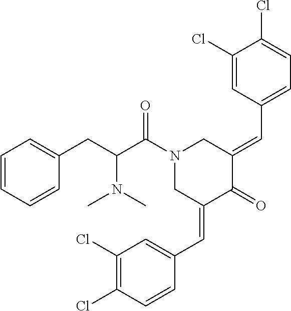 [SMILES: CN(C)C(Cc1ccccc1)C(=O)N1C/C(=C\c2ccc(Cl)c(Cl)c2)C(=O)/C(=C/c2ccc(Cl)c(Cl)c2)C1] |
| C1 | 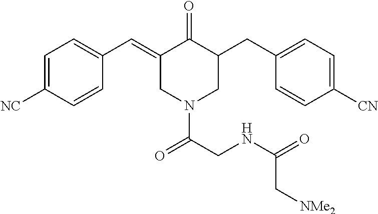 [SMILES: CN(C)CC(=O)NCC(=O)N1C/C(=C\c2ccc(C#N)cc2)C(=O)C(Cc2ccc(C#N)cc2)C1] |
| C2 | 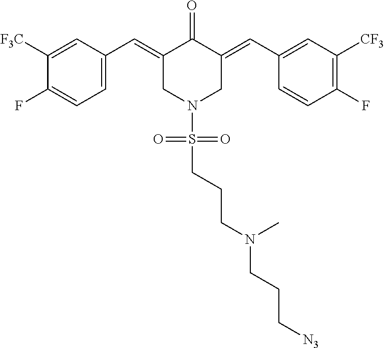 [SMILES: CN(CCCN=[N+]=[N-])CCCS(=O)(=O)N1C/C(=C\c2ccc(F)c(C(F)(F)F)c2)C(=O)/C(=C/c2ccc(F)c(C(F)(F)F)c2)C1] |
| G1 | 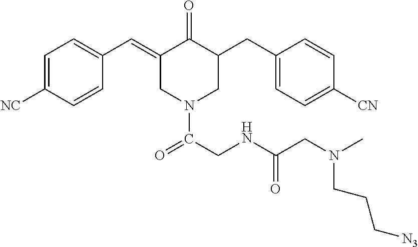 [SMILES: CN(CCCN=[N+]=[N-])CC(=O)NCC(=O)N1C/C(=C\c2ccc(C#N)cc2)C(=O)C(Cc2ccc(C#N)cc2)C1] |
| C3 | 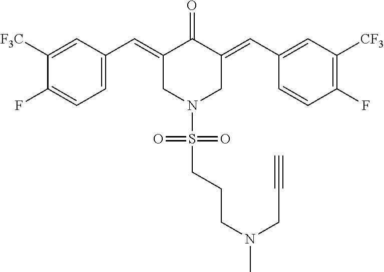 [SMILES: C#CCN(C)CCCS(=O)(=O)N1C/C(=C\c2ccc(F)c(C(F)(F)F)c2)C(=O)/C(=C/c2ccc(F)c(C(F)(F)F)c2)C1] |
| H1 | 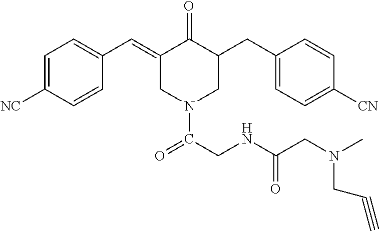 [SMILES: C#CCN(C)CC(=O)NCC(=O)N1C/C(=C\c2ccc(C#N)cc2)C(=O)C(Cc2ccc(C#N)cc2)C1] |
| D1 | 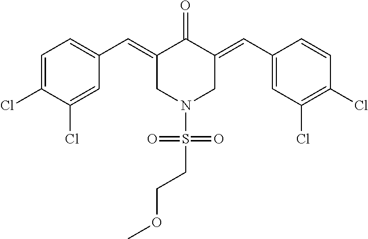 [SMILES: COCCS(=O)(=O)N1C/C(=C\c2ccc(Cl)c(Cl)c2)C(=O)/C(=C/c2ccc(Cl)c(Cl)c2)C1] |
| E1 | 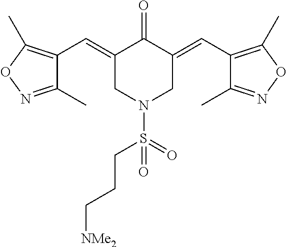 [SMILES: Cc1noc(C)c1/C=C1\CN(S(=O)(=O)CCCN(C)C)C/C(=C\c2c(C)noc2C)C1=O] |
| F1 | 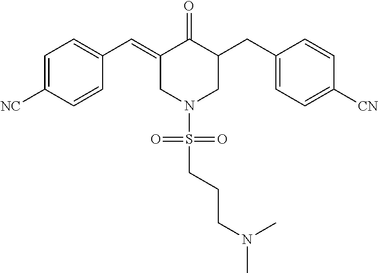 [SMILES: CN(C)CCCS(=O)(=O)N1C/C(=C\c2ccc(C#N)cc2)C(=O)C(Cc2ccc(C#N)cc2)C1] |
| B1-9 | 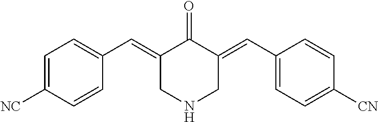 [SMILES: N#Cc1ccc(/C=C2\CNC/C(=C\c3ccc(C#N)cc3)C2=O)cc1] |
| B1-11 | 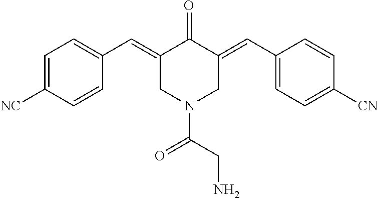 [SMILES: N#Cc1ccc(/C=C2\CN(C(=O)CN)C/C(=C\c3ccc(C#N)cc3)C2=O)cc1] |
| C1-6 | 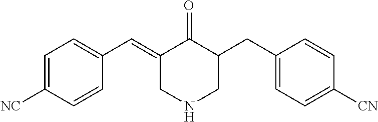 [SMILES: N#Cc1ccc(/C=C2\CNCC(Cc3ccc(C#N)cc3)C2=O)cc1] |
| C1-7 | 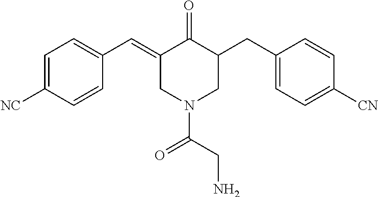 [SMILES: N#Cc1ccc(/C=C2\CN(C(=O)CN)CC(Cc3ccc(C#N)cc3)C2=O)cc1] |
| C1-8 | 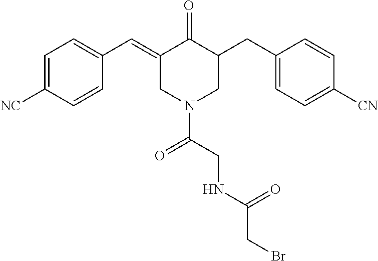 [SMILES: N#Cc1ccc(/C=C2\CN(C(=O)CNC(=O)CBr)CC(Cc3ccc(C#N)cc3)C2=O)cc1] |
| AA-8 | 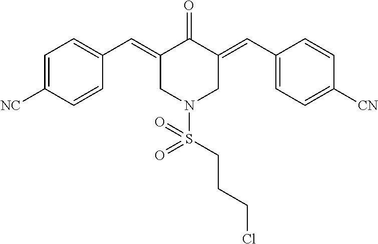 [SMILES: N#Cc1ccc(/C=C2\CN(S(=O)(=O)CCCCl)C/C(=C\c3ccc(C#N)cc3)C2=O)cc1] |
| E1-1 | 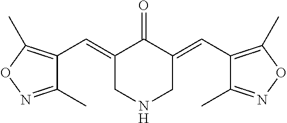 [SMILES: Cc1noc(C)c1/C=C1\CNC/C(=C\c2c(C)noc2C)C1=O] |
| E1-2 | 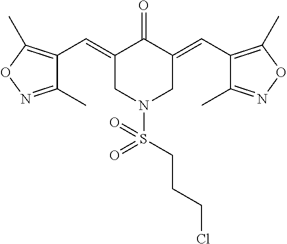 [SMILES: Cc1noc(C)c1/C=C1\CN(S(=O)(=O)CCCCl)C/C(=C\c2c(C)noc2C)C1=O] |
| F1-5 | 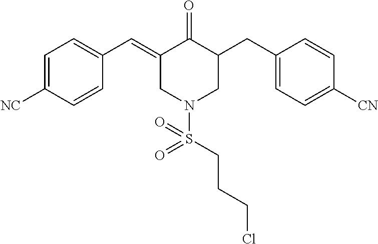 [SMILES: N#Cc1ccc(/C=C2\CN(S(=O)(=O)CCCCl)CC(Cc3ccc(C#N)cc3)C2=O)cc1] |
| CA-1 | 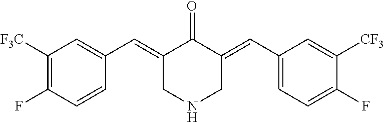 [SMILES: O=C1/C(=C/c2ccc(F)c(C(F)(F)F)c2)CNC/C1=C\c1ccc(F)c(C(F)(F)F)c1] |
| CA-2 | 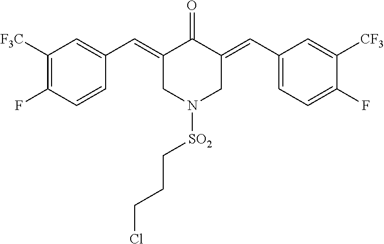 [SMILES: O=C1/C(=C/c2ccc(F)c(C(F)(F)F)c2)CN(S(=O)(=O)CCCCl)C/C1=C\c1ccc(F)c(C(F)(F)F)c1] |
| BA-2 | 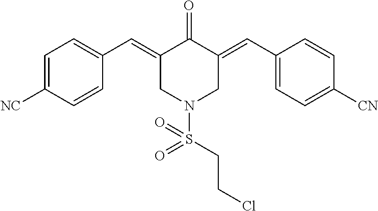 [SMILES: N#Cc1ccc(/C=C2\CN(S(=O)(=O)CCCl)C/C(=C\c3ccc(C#N)cc3)C2=O)cc1] |
| B2-7 | 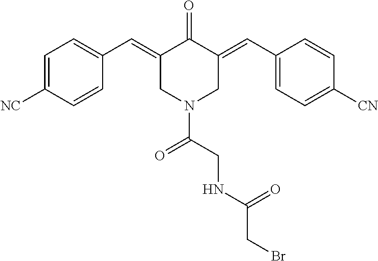 [SMILES: N#Cc1ccc(/C=C2\CN(C(=O)CNC(=O)CBr)C/C(=C\c3ccc(C#N)cc3)C2=O)cc1] |
| B5-6 | 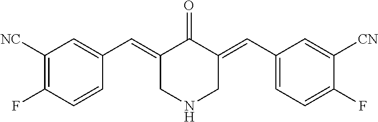 [SMILES: N#Cc1cc(/C=C2\CNC/C(=C\c3ccc(F)c(C#N)c3)C2=O)ccc1F] |

or geometrical isomer, optical isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal thereof.

In some embodiments, the compound according to this invention is represented by the structure of Compound B1:

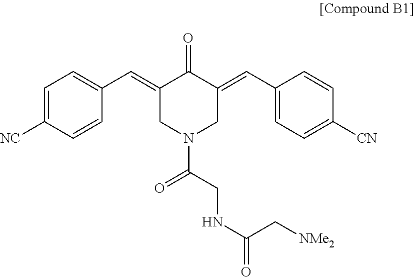 [SMILES: CN(C)CC(=O)NCC(=O)N1C/C(=C\c2ccc(C#N)cc2)C(=O)/C(=C/c2ccc(C#N)cc2)C1]

wherein the compound is a species of unreduced Compound A1, i.e., wherein both of Q₁ and Q₂ are CH, each of R₁ to R₆ is hydrogen, and R₇ and R₈ are both methyls.

In some embodiments, the compound according to this invention is represented by the structure of Compound C1, wherein the compound is a species of partially reduced Compound A-2, i.e., wherein either one of Q₁ and Q₂ in CH₂, each of R₁ to R₆ is hydrogen, and R₇ and R₈ are both methyl.

In some embodiments, the compound according to this invention is represented by the structure of Compound B2, wherein the compound is a species of unreduced Compound A1, i.e., wherein both of Q₁ and Q₂ are CH, each of R₁ to R₆ is hydrogen, and R₇ is a methyl and R₈ is an azidopropyl.

In some embodiments, the compound according to this invention is represented by the structure of Compound B3, wherein the compound is a species of unreduced Compound A1, i.e., wherein both of Q₁ and Q₂ are CH, each of R₁ to R₆ is hydrogen, and R₇ is a methyl and R₈ is a propyne.

In some embodiments, the compound according to this invention is represented by the structure of Compound B4, wherein the compound is a species of Compound of Formula II and/or III, wherein both of Q₁ and Q₂ are CH, each of R₁, R₁′, R₃, R₃′, R₄, R₄′, R₅ and R₆ is hydrogen, R₂ and R₂′ are NO₂, R₁₇ and R₁₇′ are F, G is C, T is O, n is 1, and Z is NH—C(O)—CH₃.

In some embodiments, the compound according to this invention is represented by the structure of Compound B5, wherein the compound is a species of Compound of Formula II and/or III, wherein both of Q₁ and Q₂ are CH, each of R₁, R₁′, R₃, R₃′, R₄, R₄′, R₅ and R₆ is hydrogen, R₂ and R₂′ are CN, R₁₇ and R₁₇′ are F, G is C, T is O, n is 1, and Z is NH—C(O)—CH₃.

In some embodiments, the compound according to this invention is represented by the structure of Compound B6, wherein the compound is a species of Compound A and/or Compound of Formula II and/or III, wherein both of Q₁ and Q₂ are CH, each of R₁, R₁′, R₂, R₂′, R₃, R₃′, R₄, R₄′, R₅ and R₆ is hydrogen, R₁₇ and R₁₇′ are CN, G is C, T is O, n is 1, and Z is NH—C(O)—CH₃.

In some embodiments, the compound according to this invention is represented by the structure of Compound B7, wherein the compound is a species of Compound of Formula II and/or III, wherein both of Q₁ and Q₂ are CH, each of R₁, R₁′, R₂, R₂′, R₃, R₃′, R₄, and R₄′ is hydrogen, R₅ is hydrogen and R₆ is CH₂—OH, R₁₇ and R₁₇′ are CN, G is C, T is O, n is 1, and Z is NH—C(O)—CH₃.

In some embodiments, the compound according to this invention is represented by the structure of Compound B8, wherein the compound is a species of Compound of Formula II and/or III, wherein both of Q₁ and Q₂ are CH, each of R₁, R₁′, R₂, R₂′, R₃, R₃′, R₄, R₄′, R₅ and R₆ is hydrogen, R₁₇ and R₁₇′ are CN, G is C, T is O, n is 1, and Z is NH—C(O)—R₁₅-R₁₃ and R₁₃ is OH.

In some embodiments, the compound according to this invention is represented by the structure of Compound G1, wherein the compound is a species of partially reduced Compound A-2, and/or of compound of formula I-III, wherein either one of Q₁ and Q₂ in CH₂, each of R₁, R₁′, R₂, R₂′, R₃, R₃′, R₄, R₄′, R₅ and R₆ is hydrogen, R₁₇ and R₁₇′ is CN, Z is —NH—C(O)—R₁₅—N(R₇)(R₈), R₁₅ is CH₂, and R₇ is a methyl and R₈ is an azidopropyl.

In some embodiments, the compound according to this invention is represented by the structure of Compound H1, wherein the compound is a species of partially reduced Compound A-2, and/or of compound of formula I-III, wherein either one of Q₁ and Q₂ in CH₂, each of R₁, R₁′, R₂, R₂′, R₃, R₃′, R₄, R₄′, R₅ and R₆ is hydrogen, R₁₇ and R₁₇′ is CN, Z is —NH—C(O)—R₁₅—N(R₇)(R₈), R₁₅ is CH₂, and R₇ is a methyl and R₈ is a propyne.

In some embodiments, compound of Formula IV or IV-1, is represented by the structure of Compound AA:

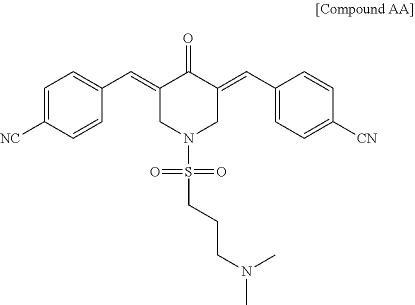

wherein R₁₀₀ is a CN substituted phenyl, and R₂₀₀ is R₁₅—N(R₁₃)(R₁₄), R₁₅ is (CH₂)₃, and R₁₃ and R₁₄ are both substituted or unsubstituted linear or branched (C₁-C₁₄) alkyl, e.g., methyl.

In some embodiments, compound of Formula IV or IV-1, is represented by the structure of Compound A2, wherein R₁₀₀ is a phenyl substituted with CN, and R₂₀₀ is R₁₅—N(R₁₃)(R₁₄), R₁₅ is (CH₂)₃, R₁₃ is a linear (C₁-C₁₄) alkyl substituted with N₃, and R₁₄ is an unsubstituted (C₁-C₁₄) alkyl, (e.g., methyl).

In some embodiments, compound of Formula IV or IV-1, is represented by the structure of Compound A3, wherein R₁₀₀ is a phenyl substituted with CN, and R₂₀₀ is R₁₅—N(R₁₃)(R₁₄), R₁₅ is (CH₂)₃, R₁₃ is a linear (C₁-C₁₄) alkyl substituted with an alkyne (e.g., propyne), and R₁₄ is an unsubstituted (C₁-C₁₄) alkyl, (e.g., methyl).

In some embodiments, compound of Formula IV or IV-1, is represented by the structure of Compound BA, wherein R₁₀₀ is a phenyl substituted with CN, R₂₀₀ is R₁₅—N(R₁₃)(R₁₄), R₁₅ is (CH₂)₂, and R₁₃ and R₁₄ are both substituted or unsubstituted linear or branched (C₁-C₁₄) alkyl, e.g., methyl.

In some embodiments, compound of Formula IV or IV-1, is represented by the structure of Compound CA, wherein R₁₀₀ is a phenyl substituted with F and CF₃, and R₂₀₀ is R₁₅—N(R₁₃)(R₁₄), R₁₅ is (CH₂)₃, and R₁₃ and R₁₄ are both substituted or unsubstituted linear or branched (C₁-C₁₄) alkyl, e.g., methyl.

In some embodiments, compound of Formula IV or IV-1, is represented by the structure of Compound C2, wherein R₁₀₀ is a phenyl substituted with F and CF₃, and R₂₀₀ is R₁₅—N(R₁₃)(R₁₄), R₁₅ is (CH₂)₃, R₁₃ is a linear (C₁-C₁₄) alkyl substituted with N₃ (e.g., propyl azide), and R₁₄ is an unsubstituted (C₁-C₁₄) alkyl, (e.g., methyl).

In some embodiments, compound of Formula IV or IV-1, is represented by the structure of Compound C3, wherein R₁₀₀ is a phenyl substituted with F and CF₃, and R₂₀₀ is R₁₅—N(R₁₃)(R₁₄), R₁₅ is (CH₂)₃, R₁₃ is a linear (C₁-C₁₄) alkyl substituted with an alkyne (e.g., propyne), and R₁₄ is an unsubstituted (C₁-C₁₄) alkyl, (e.g., methyl).

In some embodiments, compound of Formula IV or IV-1, is represented by the structure of Compound D1, wherein R₁₀₀ is a phenyl substituted with two Cl atoms (i.e., dichloro phenyl), and R₂₀₀ is R₁₅—O(R₁₃), wherein R₁₅ is (CH₂)₃, and R₁₃ is an unsubstituted linear (C₁-C₁₄) alkyl (e.g., methyl).

In some embodiments, compound of Formula IV or IV-1, is represented by the structure of Compound E1, wherein R₁₀₀ is an isoxazole substituted with two linear (C₁-C₁₄) alkyls (e.g., methyls), and R₂₀₀ is R₁₅—N(R₁₃)(R₁₄), R₁₅ is (CH₂)₃, R₁₃ and R₁₄ are both unsubstituted linear (C₁-C₁₄) alkyls (e.g., methyl).

In some embodiments, the compounds of the subject application are in the form of a geometrical isomer thereof.

For example: in some embodiments, Compound AA is in the form of a geometrical isomer thereof, represented by the structure of formulas AA-C1 or AA-C2:

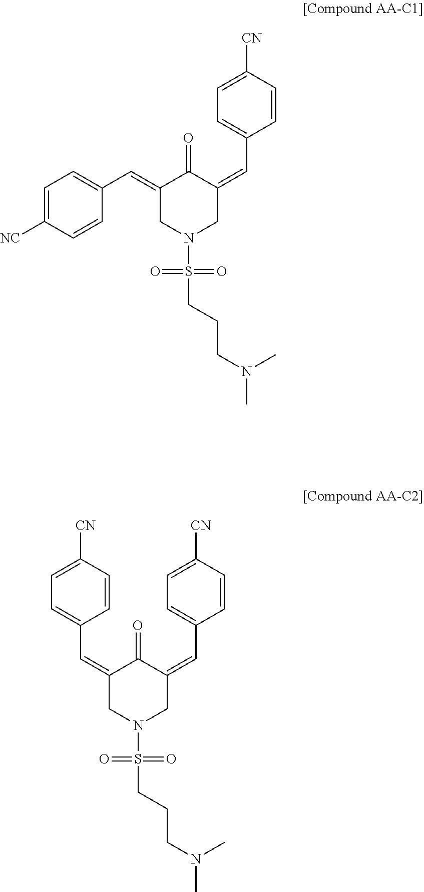 [SMILES: CN(C)CCCS(=O)(=O)N1C/C(=C/c2ccc(C#N)cc2)C(=O)/C(=C/c2ccc(C#N)cc2)C1 ; CN(C)CCCS(=O)(=O)N1C/C(=C/c2ccc(C#N)cc2)C(=O)/C(=C\c2ccc(C#N)cc2)C1]

In some embodiments, Compound D1 is in the form of a geometrical isomer thereof, represented by the structure of Compounds D1-C1 or D1-C2:

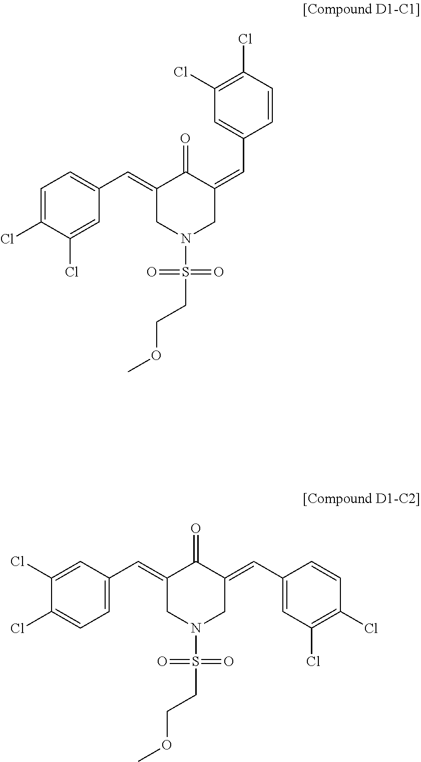 [SMILES: COCCS(=O)(=O)N1C/C(=C/c2ccc(Cl)c(Cl)c2)C(=O)/C(=C/c2ccc(Cl)c(Cl)c2)C1 ; COCCS(=O)(=O)N1C/C(=C\c2ccc(Cl)c(Cl)c2)C(=O)/C(=C/c2ccc(Cl)c(Cl)c2)C1]

In some embodiments, Compound E1 is in the form of a geometrical isomer thereof, represented by the structure of Compounds E1-C1 or E1-C2:

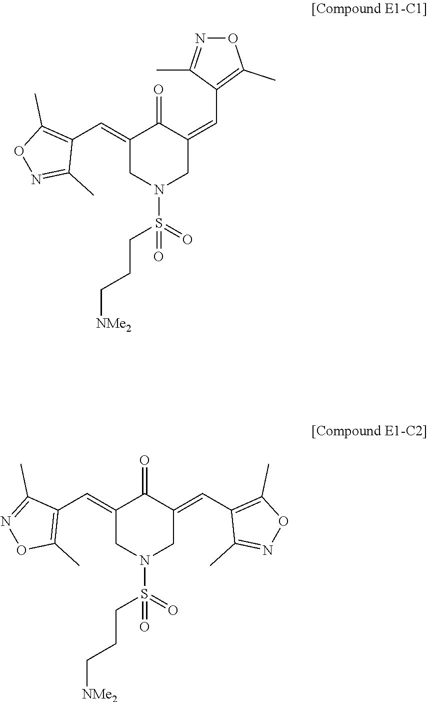 [SMILES: Cc1noc(C)c1/C=C1/CN(S(=O)(=O)CCCN(C)C)C/C(=C\c2c(C)noc2C)C1=O ; Cc1noc(C)c1/C=C1\CN(S(=O)(=O)CCCN(C)C)C/C(=C\c2c(C)noc2C)C1=O]

In some embodiments, the partially reduced form of compound of Formula IV or IV-1, is represented by the structure of Compound F1, wherein Q is CH, Q₂ is CH₂, R₁₀₀ is a phenyl substituted with CN, R₂₀₀ is R₁₅—N(R₁₃)(R₁₄), R₁₅ is (CH₂)₃, and R₁₃ and R₁₄ are both substituted or unsubstituted linear or branched (C₁-C₁₄) alkyl, e.g., methyl.

As used herein, the term “alkyl group” is meant to comprise from 1 to 30 carbon atoms, for example 1 to 3, 1 to 6, 2 to 10, 3 to 10, 2 to 8, 1 to 10, or 2 to 12 carbon atoms, which may include one or more unsaturated carbon atoms. In some embodiments, the alkyl group may be straight- or branched-chain containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₅ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. The alkyl group can be a sole substituent or it can be a component of a larger substituent, such as in an alkoxy, alkoxyalkyl, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, 2, 3, or 4-CH₂—C₆H₄—Cl, C(OH)(CH₃)(Ph), etc.

As used herein, the term “alkenyl” refers to an unsaturated hydrocarbon that contains at least one carbon-carbon double bond. In some embodiments, the alkenyl comprises from 1 to 30 carbon atoms, for example 1 to 3, 1 to 6, 2 to 10, 3 to 10, 2 to 8, 1 to 10, or 2 to 12 carbon atoms, each represents a separate embodiment according to this invention, and each comprises at least two unsaturated carbon atoms. In some embodiments, the alkenyl group may be straight- or branched-chain containing up to about 30 carbons unless otherwise specified. In various embodiments, an alkenyl includes C₁-C₅ carbons. In some embodiments, an alkenyl includes C₁-C₆ carbons. In some embodiments, an alkenyl includes C₁-C₅ carbons. In some embodiments, an alkenyl includes C₁-C₁₀ carbons. In some embodiments, an alkenyl is a C₁-C₁₂ carbons. In some embodiments, an alkenyl is a C₁-C₂₀ carbons. In some embodiments, branched alkenyl is an alkenyl substituted by alkyl side chains of 1 to 5 carbons. In various embodiments, the alkenyl group may be unsubstituted. In some embodiments, the alkenyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. The alkenyl group can be a sole substituent or it can be a component of a larger substituent, such as in an alkoxy, alkoxyalkyl, haloalkyl, arylalkyl, alkylamino, dialkylamino, alkylamido, alkylurea, etc. Preferred alkenyl groups are ethenyl (acetylene), and propenyl, and thus haloethenyl, dihaloethenyl, trihaloethenyl, halopropenyl, dihalopropenyl, trihalopropenyl, ethenoxy, propenoxy, arylethenyl, arylpropenyl, ethenylamino, propenylamino, diethenylamino, propenylamido, etc.

As used herein, the term “alkynyl” refers to an unsaturated hydrocarbon that contains at least one carbon-carbon triple bond. In some embodiments, the alkynyl comprises from 1 to 30 carbon atoms, for example 1 to 3, 1 to 6, 2 to 10, 3 to 10, 2 to 8, 1 to 10, or 2 to 12 carbon atoms, each represents a separate embodiment according to this invention, and each comprises at least two unsaturated SP carbon atoms. In some embodiments, the alkynyl group may be straight- or branched-chain containing up to about 30 carbons unless otherwise specified. In various embodiments, an alkynyl includes C₁-C₅ carbons. In some embodiments, an alkynyl includes C₁-C₆ carbons. In some embodiments, an alkynyl includes C₁-C₅ carbons. In some embodiments, an alkynyl includes C₁-C₁₀ carbons. In some embodiments, an alkynyl is a C₁-C₁₂ carbons. In some embodiments, an alkynyl includes C₁-C₂₀ carbons. In some embodiments, branched alkynyl is an alkynyl substituted by alkyl side chains of 1 to 5 carbons. In various embodiments, the alkynyl group may be unsubstituted. In some embodiments, the alkynyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. The alkynyl group can be a sole substituent or it can be a component of a larger substituent, such as in an alkoxy, alkoxyalkyl, haloalkyl, arylalkyl, alkylamino, dialkylamino, alkylamido, alkylurea, etc. Preferred alkynyl groups are ethynyl, propynyl and butynyl.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. Exemplary aryl groups include, without limitation, phenyl, tolyl, xylyl, naphthyl, phenylmethyl, phenylethyl, phenylamino, phenylamido, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or —C(O)NH₂.

As used herein, the term “heteroaryl” refers to any aromatic ring, which contain at least one heteroatom selected from O, N and S, that is directly bonded to another group and can be either substituted or unsubstituted. The heteroaryl group can be a sole substituent, or the heteroaryl group can be a component of a larger substituent, such as in an heteroarylalkyl, heteroarylamino, heteroarylamido, etc. Exemplary heteroaryl groups include, without limitation, furanyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or —C(O)NH₂.

As used herein, the term “alkoxy” refers to an ether group substituted by an alkyl group as defined above. Alkoxy refers both to linear and to branched alkoxy groups, as well as to cyclic alkoxy groups. Nonlimiting examples of alkoxy groups are methoxy, ethoxy, propoxy, iso-propoxy, tert-butoxy, cyclopropoxy, cyclobutoxy etc.

As used herein, the term “thioalkoxy” or “thioalkyl” refers to a thioether group substituted by an alkyl group as defined above (i.e., —SR). Thioalkyl refers both to linear and to branched thioalkyl groups, as well as to cyclic thioalkyl groups. Nonlimiting examples of thioalkyl groups are thiomethyl (methanthiolyl), thioethyl (ethanethiolyl), thiopropyl (or propanethiolyl), propane-2-thiolyl, 2-methylpropane-2-thiol, cyclopropanethiolyl, cyclobutanethiolyl etc.

As used herein, the term “aminoalkyl” refers to an amine group substituted by an alkyl group as defined above. Aminoalkyl refers to monoalkylamine, dialkylamine or trialkylamine. Nonlimiting examples of aminoalkyl groups are —N(Me)₂, —NHMe, —N(Et)₂.

A “haloalkyl” group refers, in some embodiments, to an alkyl group as defined above, which is substituted by one or more halogen atoms, e.g. by F, Cl, Br or I. The term “haloalkyl” include but is not limited to fluoroalkyl, i.e., to an alkyl group bearing at least one fluorine atom. Nonlimiting examples of haloalkyl groups are CF₃, CF₂CF₃, CF₂CH₃, CH₂CF₃.

An “haloalkoxy” group refers, in some embodiments, to an alkoxy group as defined above, which is substituted by one or more halogen atoms, e.g. by F, Cl, Br or I. The term “haloalkoxy” include but is not limited to fluoroalkoxy, i.e., to an alkoxy group bearing at least one fluorine atom. Nonlimiting examples of haloalkoxy groups are OCF₃, OCF₂CF₃, OCF₂CH₃, OCH₂CF₃ etc.

An “alkoxyalkyl” group refers, in some embodiments, to an alkyl group as defined above, which is substituted by alkoxy group as defined above, e.g. by methoxy, ethoxy, propoxy, i-propoxy, t-butoxy etc. Nonlimiting examples of alkoxyalkyl groups are —CH₂—O—CH₃, —CH₂—O—CH(CH₃)₂, —CH₂—O—C(CH₃)₃, —CH₂—CH₂—O—CH₃, —CH₂—CH₂—O—CH(CH₃)₂, —CH₂—CH₂—O—C(CH₃)₃.

A “cycloalkyl” or “carbocyclic” group refers, In various embodiments, to a ring structure comprising carbon atoms as ring atoms, which may be either saturated or unsaturated, substituted or unsubstituted, single or fused. In some embodiments the cycloalkyl is a 3-10 membered ring. In some embodiments the cycloalkyl is a 3-12 membered ring. In some embodiments the cycloalkyl is a 6 membered ring. In some embodiments the cycloalkyl is a 5-7 membered ring. In some embodiments the cycloalkyl is a 3-8 membered ring. In some embodiments, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In some embodiments, the cycloalkyl ring may be fused to another saturated or unsaturated cycloalkyl or heterocyclic 3-8 membered ring. In some embodiments, the cycloalkyl ring is a saturated ring. In some embodiments, the cycloalkyl ring is an unsaturated ring. Non limiteing examples of a cycloalkyl group comprise cyclohexyl, cyclohexenyl, cyclopropyl, cyclopropenyl, cyclopentyl, cyclopentenyl, cyclobutyl, cyclobutenyl, cycloctyl, cycloctadienyl (COD), cycloctaene (COE) etc.

A “heterocycle” or “heterocyclic” group refers, in various embodiments, to a ring structure comprising in addition to carbon atoms, sulfur, oxygen, nitrogen or any combination thereof, as part of the ring. A “heteroaromatic ring” refers in various embodiments, to an aromatic ring structure comprising in addition to carbon atoms, sulfur, oxygen, nitrogen or any combination thereof, as part of the ring. In some embodiments the heterocycle or heteroaromatic ring is a 3-10 membered ring. In some embodiments the heterocycle or heteroaromatic ring is a 3-12 membered ring. In some embodiments the heterocycle or heteroaromatic ring is a 6 membered ring. In some embodiments the heterocycle or heteroaromatic ring is a 5-7 membered ring. In some embodiments the heterocycle or heteroaromatic ring is a 3-8 membered ring. In some embodiments, the heterocycle group or heteroaromatic ring may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In some embodiments, the heterocycle ring or heteroaromatic ring may be fused to another saturated or unsaturated cycloalkyl or heterocyclic 3-8 membered ring. In some embodiments, the heterocyclic ring is a saturated ring. In some embodiments, the heterocyclic ring is an unsaturated ring. Non limiting examples of a heterocyclic ring or heteroaromatic ring systems comprise pyridine, piperidine, morpholine, piperazine, thiophene, pyrrole, benzodioxole, benzofuran-2(3H)-one, benzo[d][1,3]dioxole or indole.

As used herein, the term “pharmaceutically acceptable carrier” refers to a carrier or adjuvant that may be administered to a subject (e.g., a patient), together with a compound of this invention, and which does not destroy the pharmacological activity thereof and is nontoxic when administered in doses sufficient to deliver a therapeutic amount or an effective amount of the compound. “Pharmaceutically acceptable carrier” refers to any and all solvents, dispersion media. The use of such media and compounds for pharmaceutically active substances is well known in the art. In some embodiments, the carrier is suitable for oral, intravenous, intramuscular, subcutaneous, parenteral, spinal or epidural administration (e.g., by injection or infusion).

In various embodiments, this invention provides a compound of this invention or its isomer, solvate, metabolite, pharmaceutically acceptable salt, pharmaceutical product, tautomer, hydrate, N-oxide, prodrug, isotopic variant, PROTAC, polymorph, crystal or combinations thereof. In various embodiments, this invention provides an isomer of the compound of this invention. In some embodiments, this invention provides a metabolite of the compound of this invention. In some embodiments, this invention provides a pharmaceutically acceptable salt of the compound of this invention. In some embodiments, this invention provides a pharmaceutical product of the compound of this invention. In some embodiments, this invention provides a tautomer of the compound of this invention. In some embodiments, this invention provides a hydrate of the compound of this invention. In some embodiments, this invention provides an N-oxide of the compound of this invention. In some embodiments, this invention provides a prodrug of the compound of this invention. In some embodiments, this invention provides an isotopic variant (including but not limited to deuterated analog) of the compound of this invention. In some embodiments, this invention provides a PROTAC (Proteolysis targeting chimera) of the compound of this invention. In some embodiments, this invention provides a polymorph of the compound of this invention. In some embodiments, this invention provides a crystal of the compound of this invention. In some embodiments, this invention provides composition comprising a compound of this invention, as described herein, or, In some embodiments, a combination of an isomer, metabolite, pharmaceutically acceptable salt, pharmaceutical product, tautomer, hydrate, N-oxide, prodrug, isotopic variant, PROTAC, polymorph, or crystal of the compound of this invention.

In various embodiments, the term “isomer” includes, but is not limited to, geometrical isomers, optical isomers, structural isomers, conformational isomers, and the like. In some embodiments, the isomer is a geometrical isomer (e.g., E-Z, cis-trans etc.). In some embodiments, the isomer is an optical isomer.

As used herein, the term “geometric isomers” refers to “cis-trans isomers”, “E-Z isomers”, or to “configurational isomers”. Geometric isomers are stereoisomers, that is, pairs of molecules which have the same formula but whose functional groups are rotated into a different orientation in three-dimensional space. In general, geometric isomers contain double bonds that do not rotate, or they may contain ring structures, where the rotation of bonds is restricted or prevented. In some embodiments, geometric isomers refer to cis-trans isomers. In other embodiments, geometric isomers refer to E-Z isomers.

In various embodiments, this invention encompasses the use of various optical isomers of the compounds of the invention. It will be appreciated by those skilled in the art that the compounds of the present invention may contain at least one chiral center. Accordingly, the compounds used in the methods of the present invention may exist in, and be isolated in, optically-active or racemic forms. Accordingly, the compounds according to this invention may exist as optically-active isomers (enantiomers or diastereomers, including but not limited to: the (R), (S), (R)(R), (R)(S), (S)(S), (S)(R), (R)(R)(R), (R)(R)(S), (R)(S)(R), (S)(R)(R), (R)(S)(S), (S)(R)(S), (S)(S)(R) or (S)(S)(S) isomers); as racemic mixtures, or as enantiomerically enriched mixtures. Some compounds may also exhibit polymorphism. It is to be understood that the present invention encompasses any racemic, optically-active, polymorphic, or stereroisomeric form, or mixtures thereof, which form possesses properties useful in the treatment of the various conditions described herein.

It is well known in the art how to prepare optically-active forms (for example, by resolution of the racemic form by recrystallization techniques, by synthesis from optically-active starting materials, by chiral synthesis, or by chromatographic separation using a chiral stationary phase).

The compounds of the present invention can also be present in the form of a racemic mixture, containing substantially equivalent amounts of stereoisomers. In some embodiments, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 95% pure, more preferably at least about 98% pure, most preferably at least about 99% pure.

Compounds of the present invention can also be in the form of a solvate, which means that the compound further includes a stoichiometric or non-stoichiometric amount of solvent bound by non-covalent intermolecular forces.

Compounds of the present invention can also be in the form of a hydrate, which means that the compound further includes a stoichiometric or non-stoichiometric amount of water bound by non-covalent intermolecular forces.

Compounds of the present invention may exist in the form of one or more of the possible tautomers and depending on the particular conditions it may be possible to separate some or all of the tautomers into individual and distinct entities. It is to be understood that all of the possible tautomers, including all additional enol and keto tautomers and/or isomers are hereby covered. For example the following tautomers, but not limited to these, are included:

Tautomerization of the Imidazole Ring

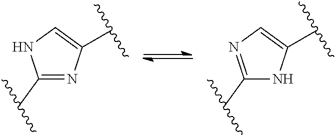 [SMILES: *c1c[nH]c(*)n1 ; *c1cnc(*)[nH]1]

Tautomerization of the Pyrazolone Ring:

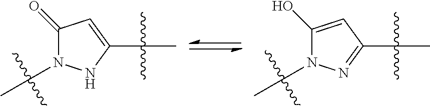 [SMILES: *c1cc(=O)n(*)[nH]1 ; *c1cc(O)n(*)n1]

The invention includes “pharmaceutically acceptable salts” of the compounds of this invention, which may be produced, by reaction of a compound of this invention with an acid or base. Certain compounds, particularly those possessing acid or basic groups, can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

In some embodiments, pharmaceutically acceptable salts of compounds described herein include but are not limited to: phosphate salt, methane sulfonate salt, hydrochloride salt, sulphate salt, citrate salt, and p-toluene sulfonate salt.

Suitable pharmaceutically-acceptable salts of amines of compounds the compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethaminesand ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

In some embodiments, pharmaceutically acceptable salts of compounds according to this invention include, without limitation, phosphate, methane sulfonate, hydrochloride, sulphate, citrate, and p-toluene sulfonate salts.

### Pharmaceutical Composition

Another aspect of the present invention relates to a pharmaceutical composition including a pharmaceutically acceptable carrier and a compound according to the aspects of the present invention. The pharmaceutical composition can contain one or more of the above-identified compounds of the present invention. Typically, the pharmaceutical composition of the present invention will include a compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In some embodiments, these compounds are tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, these active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

In some embodiments, the administration is via intraperitoneal injection. In some embodiments, the administration is via intravenous injection. In some embodiments, the intravenous injection is by bolus injection or infusion injection. In some embodiments, the administration is via subcutaneous injection. In some embodiments, the administration is oral.

For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Aspects of the invention relate to pharmaceutical compositions comprising one or more of the compounds described herein. In some embodiments, the pharmaceutical compositions comprise one or more of the following: pharmaceutically acceptable adjuvant, diluent, excipient, and carrier. In some embodiments, the pharmaceutical composition comprises one or more of the compounds described herein in combination with one or more therapeutic agents.

In various embodiments, the compounds of this invention are administered in combination with an anti-cancer agent. In various embodiments, the anti-cancer agent is a proteasome inhibitor.

In some embodiments, the pharmaceutical composition comprising compounds according to this invention, may be combined with a drug for treating multiple myeloma. In some embodiments, examples of the drug for treatment multiple myeloma can include, but are not limited to, proteasome inhibitors (e.g., but not limited to bortezomib, carfilzomib, etc.), immune-modifying drugs (IMiDs) (e.g., but not limited to, thalidomide, lenalidomide, pomalidomide, etc.), monoclonal antibodies (mAbs) (e.g., but not limited to, elotuzumab, daratumumab, MOR03087, isatuximab, bevacizumab, cetuximab, siltuximab, tocilizumab, elsilimomab, azintrel, rituximab, tositumomab, milatuzumab, lucatumumab, dacetuzumab, figitumumab, dalotuzumab, AVE1642, tabalumab, pembrolizumab, pidilizumab, nivolumab, which are described in Zagouri et al., Expert Opin Emerg Drugs (2016) June:21(2):225-37, which is hereby incorporated by reference in its entirety), chemotherapy (e.g., but not limited to, dexamethasone, melphalan, doxorubicin, cyclophosphamide, etc.), histone deacetylase inhibitors (e.g., but not limited to, Vorinostat and Panobinostat, as disclosed in Cea et al., Curr Pharm Des (2013); 19(4): 734-744, which is hereby incorporated by reference in its entirety).

In various embodiments, the compounds of this invention are administered in combination with at least one of the following: chemotherapy, radiation therapy, biological therapy, molecularly-targeted therapies, DNA damaging agents, hypoxia-inducing agents, or immunotherapy, each possibility represents a separate embodiment of this invention. Chemotherapy drug includes, for example, alkylating agents, nitrosourea agents, antimetabolites, antitumor antibiotics, alkaloids derived from plant, topoisomerase inhibitors, hormone therapy medicines, hormone antagonists, aromatase inhibitors, P-glycoprotein inhibitors, platinum complex derivatives, other immunotherapeutic drugs, and other anticancer agents. Further, they can be used together with hypoleukocytosis (neutrophil) medicines that are cancer treatment adjuvant, thrombopenia medicines, antiemetic drugs, and cancer pain medicines for patient's QOL recovery or be made as a mixture with them.

When administering the compounds of the present invention, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biological Activity

In various embodiments, the compounds according to this invention exhibit cytotoxicity upon exposure to a variety of cancer cells. In some embodiments, the compounds according to this invention inhibit the Ubiquitin Proteasome System (UPS). In some embodiments, the compounds according to this invention induce the accumulation of poly-ubiquinated proteins in cells treated therewith. In some embodiments, the compounds according to this invention do not inhibit the proteasomal activity. In some embodiments, the compounds according to this invention do not inhibit the enzymatic functions of the proteasome. In some embodiments, the compounds according to this invention have a mechanism of action that is different from the proteasomes inhibitors. In some embodiments, the compounds according to this invention inhibit protein degradation.

In some embodiments, the present invention is directed to a method for reducing the growth of at least one tumor in a subject in need thereof comprising: administering a therapeutically effective amount of a compound according to this invention, for a sufficient period of time so as to result in reducing growth by at least 10 percent, compared to an untreated tumor or a tumor treated with a vehicle (i.e., a carrier or excipient) without (i.e., in the absence of) the compound described herein.

As used herein, the term “tumor” includes both solid and non-solid malignancies.

In some embodiments, the method comprises administering a composition comprising a therapeutically effective amount of a compound according to this invention. In some embodiments, the method reduces tumor growth by at least 20 percent, by at least 30 percent, by at least 40 percent, by at least 50 percent, by at least 60 percent, by at least 70 percent, by at least 80 percent, by at least 90 percent, by at least 95 percent, by at least 99 percent, by up to 100 percent of the at least one tumor in the subject, compared to an untreated tumor or a tumor treated with the vehicle without the compounds described herein; each represents a separate embodiment according to this invention.

In some embodiments, the present invention is directed to a method for reducing growth of at least one tumor in a subject comprising: obtaining a compound according to this invention, and administering a therapeutically effective amount thereof for a sufficient period of time so as to result in reducing growth by at least 10 percent compared to an untreated tumor or a tumor treated with the vehicle without the compounds described herein. In some embodiments, the method reduces tumor growth by at least 20 percent, by at least 30 percent, by at least 40 percent, by at least 50 percent, by at least 60 percent, by at least 70 percent, by at least 80 percent, by at least 90 percent, by at least 95%, by at least 99 percent, by up to 100 percent of the at least one tumor in the subject, compared to an untreated tumor or a tumor treated with the vehicle without the compounds described herein; each represents a separate embodiment according to this invention. In some embodiments, the method comprises administering a pharmaceutical composition comprising a therapeutically effective amount of a compound according to this invention. In some embodiments, the tumor is a solid tumor. In some embodiments, the tumor is SMARCB1-deficient tumor.

As used herein, the term “reducing tumor growth” is also intended to encompass inhibiting tumor growth or cancer growth which includes the prevention of the growth of a tumor in a subject or a reduction in the growth of a pre-existing tumor in a subject. A cancer is “inhibited” if at least one symptom of the cancer is alleviated, terminated, slowed, or prevented. As used herein, cancer is also “inhibited” if recurrence of the cancer is reduced, slowed, delayed, or prevented.

In some embodiments, compounds according to this invention, and method or use thereof, reduce the tumor growth in a subject by about 10 percent to 70 percent, 10 percent to 80 percent, 10 percent to 90 percent, 10 percent to 100 percent compared to an untreated tumor or a tumor treated with the vehicle without the compounds described herein; each represents a separate embodiment according to this invention.

In some embodiments, the at least one tumor is a malignant tumor. In some embodiments, the malignant tumor is a cancer. In some embodiments, for example without limitation, the cancer can be a multiple myeloma, breast cancer, colon cancer, colorectal cancer, leukemia, lymphoma, lung cancer, ovarian cancer, cervical cancer, uterine cancer, renal cancer, prostate cancer, melanoma, bone cancer and CNS cancer. In some embodiments, the cancer is multiple myeloma (MM). In some embodiments, the cancer is multiple myeloma refractory to proteasome inhibitors.

In various embodiments, this invention is directed to a method of treating, suppressing, reducing the severity, reducing the risk of developing or inhibiting cancer comprising administering a compound of this invention to a subject suffering from cancer under conditions effective to treat, suppress, reduce the severity, reduce the risk of developing, or inhibit the cancer. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the cancer is early cancer. In some embodiments, the cancer is advanced cancer. In some embodiments, the cancer is invasive cancer. In some embodiments, the cancer is metastatic cancer. In some embodiments, the cancer is drug resistant cancer. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In some embodiments, the cancer is drug resistant cancer. In some embodiments, the cancer is selected from: Multiple myeloma, bladder cancer, Myelodysplasia, breast cancer, cervix cancer, endometrium cancer, esophagus cancer, head and neck cancer (squamous cell carcinoma), kidney cancer (renal cell carcinoma), liver cancer (hepatocellular carcinoma), lung cancer (non-small cell; NSCLC), nasopharynx cancer, solid tumor cancer, stomach cancer, adrenocortical carcinoma, Glioblastoma multiforme, acute myeloid Leukemia, chronic lymphocytic Leukemia, Hodgkin's (classical) Lymphoma, diffuse large B-cell Lymphoma, primary central nervous system Lymphoma, malignant Melanoma, uveal Melanoma, Meningioma, breast cancer, anus cancer, anus (squamous cell) cancer, biliary cancer, bladder cancer, muscle invasive urothelial carcinoma, colorectal cancer, fallopian tube cancer, gastroesophageal junction cancer, larynx (squamous cell) cancer, lung cancer (small cell, SCLC), merkel cell cancer, mouth cancer, ovary cancer, pancreas cancer, penis cancer, peritoneum cancer, prostate cancer, rectum cancer, skin cancer (basal cell carcinoma, squamous cell carcinoma), small intestine cancer, testis cancer, thymus cancer, anaplastic thyroid cancer, Cholangiocarcinoma, Chordoma, Cutaneous T-cell lymphoma, Digestive-gastrointestinal cancer, Familial pheochromocytoma-paraganglioma, Glioma, HTLV-1-associated adult T-cell leukemia-lymphoma, Hematologic-blood cancer, uterine Leiomyosarcoma, acute lymphocytic Leukemia, chronic myeloid Leukemia, T-cell Lymphoma, follicular Lymphoma, primary mediastinal large B-cell Lymphoma, testicular diffuse large B-cell Lymphoma, Melanoma, malignant Mesothelioma, pleural Mesothelioma, Mycosis fungoides, Neuroendocrine cancer, Oral epithelial dysplasia, Sarcoma, Uterine cancer, myeloma Smoldering, Soft tissue sarcoma, nasal natural killer (NK) cell T-cell lymphoma and peripheral T-cell lymphoma; each represents a separate embodiment according to this invention.

In some embodiments, for example without limitation, the cancer can be a multiple myeloma, breast cancer, colon cancer, colorectal cancer, leukemia, lymphoma, lung cancer, ovarian cancer, cervical cancer, uterine cancer, renal cancer, prostate cancer, melanoma, bone cancer and CNS cancer. In some embodiments, the compound is any one of the compounds listed in Table A; each compound represents a separate embodiment according to this invention.

In some embodiments, the cancer is selected from: Acute monocytic leukemia, Acute myeloid leukemia, T acute lymphoblastic leukemia, Alveolar rhabdomyosarcoma, Melanoma, Amelanotic melanoma, Cutaneous melanoma, Anaplastic large cell lymphoma, Diffuse large B-cell lymphoma, T lymphoblastic lymphoma, Astrocytoma, B acute lymphoblastic leukemia, Biphasic synovial sarcoma, Bladder carcinoma, Breast Cancer, Breast carcinoma, Breast adenocarcinoma, Cecum adenocarcinoma, Cervical carcinoma, Cervical squamous cell carcinoma, Chronic myelogenous leukemia, CNS cancer, Colon cancer, Colon carcinoma, Colon adenocarcinoma, Duodenal adenocarcinoma, Embryonal rhabdomyosarcoma, Endometrial adenocarcinoma, Endometrial adenosquamous carcinoma, Epithelioid sarcoma, Fibrosarcoma, Gastric adenocarcinoma, Gastric carcinoma, Signet ring cell gastric adenocarcinoma, Gestational choriocarcinoma, Glioblastoma, Hereditary thyroid gland medullary carcinoma, Hypopharyngeal squamous cell carcinoma, Invasive ductal carcinoma, Liposarcoma, Lung cancer, Large cell lung carcinoma, Lung adenocarcinoma, Small cell lung carcinoma, Squamous cell lung carcinoma, Neuroblastoma, Osteosarcoma, Ovarian cancer, Ovarian clear cell adenocarcinoma, Ovarian mixed germ cell tumor, High grade ovarian serous adenocarcinoma, Uterine cancer, Pancreatic adenocarcinoma, Pancreatic ductal adenocarcinoma, Papillary renal cell carcinoma, Primitive neuroectodermal tumor, Prostate carcinoma, Rectal adenocarcinoma, Medulloblastoma, Renal cancer, Renal cell carcinoma, Testicular embryonal carcinoma and Tongue squamous cell carcinoma; each represents a separate embodiment according to this invention. In some embodiments, the compound is any one of the compounds listed in Table A; each compound represents a separate embodiment according to this invention.

Accordingly, in various embodiments, this invention is directed to a method of treating, suppressing, reducing the severity, reducing the risk of developing or inhibiting multiple myeloma (MM) comprising administering a compound of this invention to a subject suffering from multiple myeloma (MM) under conditions effective to treat, suppress, reduce the severity, reduce the risk of developing, or inhibit the multiple myeloma (MM). In some embodiments, the multiple myeloma (MM) is early multiple myeloma (MM). In some embodiments, the multiple myeloma (MM) is advanced multiple myeloma (MM). In some embodiments, the multiple myeloma (MM) is invasive multiple myeloma (MM). In some embodiments, the multiple myeloma (MM) is metastatic multiple myeloma (MM). In some embodiments, the multiple myeloma (MM) is drug resistant multiple myeloma (MM). In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting leukemia comprising administering a compound of this invention to a subject suffering from leukemia under conditions effective to treat, suppress, reduce the severity, reduce the risk of developing, or inhibit the leukemia. In some embodiments, the leukemia is early. In some embodiments, the leukemia is advanced. In some embodiments, the leukemia is invasive. In some embodiments, the leukemia is metastatic. In some embodiments, the leukemia is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting lymphoma comprising administering a compound of this invention to a subject suffering from lymphoma under conditions effective to treat, suppress, reduce the severity, reduce the risk of developing, or inhibit the lymphoma. In some embodiments, the lymphoma is B-cell non-Hodgkin's lymphoma (NHL). In some embodiments, the lymphoma is Mantle cell lymphoma (MCL). In some embodiments, the lymphoma is early. In some embodiments, the lymphoma is advanced. In some embodiments, the lymphoma is invasive. In some embodiments, the lymphoma is metastatic. In some embodiments, the lymphoma is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting Monoclonal gammopathy of undetermined significance (MGUS) comprising administering a compound of this invention to a subject suffering from MGUS under conditions effective to treat, suppress, reduce the severity, reduce the risk of developing, or inhibit the MGUS. In some embodiments, the MGUS is early. In some embodiments, the MGUS is advanced. In some embodiments, the MGUS is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting breast cancer comprising administering a compound of this invention to a subject suffering from breast cancer under conditions effective to treat, suppress, reduce the severity, reduce the risk of developing, or inhibit the breast cancer. In some embodiments, the breast cancer is early. In some embodiments, the breast cancer is advanced. In some embodiments, the breast cancer is invasive. In some embodiments, the breast cancer is metastatic. In some embodiments, the breast cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting ovarian cancer comprising administering a compound of this invention to a subject suffering from ovarian cancer under conditions effective to treat, suppress, reduce the severity, reduce the risk of developing, or inhibit the ovarian cancer. In some embodiments, the ovarian cancer is early. In some embodiments, the ovarian cancer is advanced. In some embodiments, the ovarian cancer is invasive. In some embodiments, the ovarian cancer is metastatic. In some embodiments, the ovarian cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting cervical cancer comprising administering a compound of this invention to a subject suffering from cervical cancer under conditions effective to treat, suppress, reduce the severity, reduce the risk of developing, or inhibit the cervical cancer. In some embodiments, the cervical cancer is early. In some embodiments, the cervical cancer is advanced. In some embodiments, the cervical cancer is invasive. In some embodiments, the cervical cancer is metastatic. In some embodiments, the cervical cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting uterine cancer comprising administering a compound of this invention to a subject suffering from uterine cancer under conditions effective to treat, suppress, reduce the severity, reduce the risk of developing, or inhibit the uterine cancer. In some embodiments, the uterine cancer is early. In some embodiments, the uterine cancer is advanced. In some embodiments, the uterine cancer is invasive. In some embodiments, the uterine cancer is metastatic. In some embodiments, the uterine cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting colon cancer comprising administering a compound of this invention to a subject suffering from colon cancer under conditions effective to treat, suppress, reduce the severity, reduce the risk of developing, or inhibit the colon cancer. In some embodiments, the colon cancer is early. In some embodiments, the colon cancer is advanced. In some embodiments, the colon cancer is invasive. In some embodiments, the colon cancer is metastatic. In some embodiments, the colon cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting colorectal cancer comprising administering a compound of this invention to a subject suffering from colorectal cancer under conditions effective to treat, suppress, reduce the severity, reduce the risk of developing, or inhibit the colorectal cancer. In some embodiments, the colorectal cancer is early. In some embodiments, the colorectal cancer is advanced. In some embodiments, the colorectal cancer is invasive. In some embodiments, the colorectal cancer is metastatic. In some embodiments, the colorectal cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting renal cancer comprising administering a compound of this invention to a subject suffering from renal cancer under conditions effective to treat, suppress, reduce the severity, reduce the risk of developing, or inhibit the renal cancer. In some embodiments, the renal cancer is early. In some embodiments, the renal cancer is advanced. In some embodiments, the renal cancer is invasive. In some embodiments, the renal cancer is metastatic. In some embodiments, the renal cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting prostate cancer comprising administering a compound of this invention to a subject suffering from prostate cancer under conditions effective to treat, suppress, reduce the severity, reduce the risk of developing, or inhibit the prostate cancer. In some embodiments, the prostate cancer is early. In some embodiments, the prostate cancer is advanced. In some embodiments, the prostate cancer is invasive. In some embodiments, the prostate cancer is metastatic. In some embodiments, the prostate cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting bone cancer comprising administering a compound of this invention to a subject suffering from bone cancer under conditions effective to treat, suppress, reduce the severity, reduce the risk of developing, or inhibit the bone cancer. In some embodiments, the bone cancer is early. In some embodiments, the bone cancer is advanced. In some embodiments, the bone cancer is invasive. In some embodiments, the bone cancer is metastatic. In some embodiments, the bone cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting central nervous system (CNS) cancer comprising administering a compound of this invention to a subject suffering from CNS cancer under conditions effective to treat, suppress, reduce the severity, reduce the risk of developing, or inhibit the CNS cancer. In some embodiments, the CNS cancer is early. In some embodiments, the CNS cancer is advanced. In some embodiments, the CNS cancer is invasive. In some embodiments, the CNS cancer is metastatic. In some embodiments, the CNS cancer is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting melanoma comprising administering a compound of this invention to a subject suffering from melanoma under conditions effective to treat, suppress, reduce the severity, reduce the risk of developing, or inhibit the melanoma. In some embodiments, the melanoma is early. In some embodiments, the melanoma is advanced. In some embodiments, the melanoma is invasive. In some embodiments, the melanoma is metastatic. In some embodiments, the melanoma is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of suppressing, reducing or inhibiting tumor growth in a subject, comprising administering a compound according to this invention, to a subject suffering from a proliferative disorder (e.g., cancer) under conditions effective to suppress, reduce or inhibit said tumor growth in said subject. In various embodiments, the tumor is SMARCB1-deficient tumor. In various embodiments, the tumor is a solid tumor.

In various embodiments, this invention is directed to a method of treating, suppressing, reducing the severity, reducing the risk of developing or inhibiting a plasma cell disorder comprising administering a compound of this invention to a subject suffering from a plasma cell disorder under conditions effective to treat, suppress, reduce the severity, reduce the risk of developing, or inhibit the plasma cell disorder. In some embodiments, the plasma cell disorder is Monoclonal Gammopathy of Undetermined Significance (MGUS), smoldering multiple myeloma (SMM), Asymptomatic Plasma Cell Myeloma, Multiple myeloma (MM), Waldenstrom's macroglobulinemia (WM), immunoglobulin light chain (AL) amyloidosis, POEMS syndrome, plasma cell (PC) leukemia, Plasmacytoma, Primary amyloidosis, or any combination thereof. In some embodiments, the plasma cell disorder is Monoclonal Gammopathy of Undetermined Significance (MGUS). In some embodiments, the plasma cell disorder is Asymptomatic Plasma Cell Myeloma. In some embodiments, the plasma cell disorder is Multiple myeloma (MM). In some embodiments, the plasma cell disorder is plasma cell (PC) leukemia. In some embodiments, the plasma cell disorder is Plasmacytoma. In some embodiments, the plasma cell disorder is Primary amyloidosis. In some embodiments, the plasma cell disorder is smoldering multiple myeloma (SMM). In some embodiments, the plasma cell disorder is Waldenstrom's macroglobulinemia (WM). In some embodiments, the plasma cell disorder is immunoglobulin light chain (AL) amyloidosis. In some embodiments, the plasma cell disorder is POEMS syndrome. In some embodiments, the plasma cell disorder is malignant. In some embodiments, the plasma cell disorder is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting a Non-plasma-cell hematologic malignancy in a subject, comprising administering a compound according to this invention to a subject suffering from Non-plasma-cell hematologic malignancy under conditions effective to treat, suppress, reduce the severity, reduce the risk of developing, or inhibit said Non-plasma-cell hematologic malignancy. In various embodiments, the Non-plasma-cell hematologic malignancy is a B-cell non-Hodgkin's lymphoma (NHL) such as Mantle cell lymphoma (MCL). In various embodiments, the Non-plasma-cell hematologic malignancy is Mantle cell lymphoma (MCL). In various embodiments, the Non-plasma-cell hematologic malignancy is a B-cell non-Hodgkin's lymphoma (NHL). In some embodiments, the Non-plasma-cell hematologic malignancy is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting a hematologic condition comprising administering a compound according to this invention to a subject suffering from hematologic condition under conditions effective to treat, suppress, reduce the severity, reduce the risk of developing, or inhibit said hematologic condition. In various embodiments, the hematologic conditions is AL Amyloidosis. In various embodiments, the hematologic conditions is post-transplant lymphoproliferative disease (PTLD). In some embodiments, the hematologic condition is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting a SMARCB1-deficient malignancy in a subject, comprising administering a compound according to this invention to a subject suffering from a SMARCB1-deficient malignancy under conditions effective to treat, suppress, reduce the severity, reduce the risk of developing, or inhibit said SMARCB1-deficient malignancy. In some embodiments, the SMARCB-deficient malignancy is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention is directed to a method of treating, suppressing, reducing the severity, reducing the risk of developing or inhibiting a post-transplant lymphoproliferative disease (PTLD) comprising administering a compound of this invention to a subject suffering from a PTLD under conditions effective to treat, suppress, reduce the severity, reduce the risk of developing, or inhibit the PTLD. In some embodiments, the PTLD is B cell lymphoma, T cell lymphoma, plasmacytoma, pediatric plasmacytoma-like PTLD, or any combination thereof. In some embodiments, the PTLD is B cell lymphoma. In some embodiments, the PTLD is T cell lymphoma. In some embodiments, the PTLD is plasmacytoma. In some embodiments, the PTLD is pediatric plasmacytoma-like PTLD. In some embodiments, the PTLD is polymorphic PTLD. In some embodiments, the PTLD is monomorphic PTLD. In some embodiments, the PTLD is classical Hodgkin-lymphoma-type PTLD. In some embodiments, the PTLD is drug resistant. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the compound is Compound AA. In some embodiments, the compound is Compound B1. In some embodiments, the compound is any one of the compounds listed in Table A; each compound represents a separate embodiment according to this invention. In some embodiments, the compound induces accumulation of poly-ubiquitinated proteins in cells treated therewith. In some embodiments, the compound disrupts autophagosomal flux in cells treated therewith. In some embodiments, the compound induces proteotoxic stress and UPR by modulating protein degradation pathways and disrupting protein homeostasis.

In various embodiments, this invention provides methods for treating, suppressing, reducing the severity, reducing the risk, or inhibiting metastatic cancer comprising the step of administering to said subject a compound of this invention and/or an isomer, solvate, metabolite, pharmaceutically acceptable salt, pharmaceutical product, tautomer, hydrate, N-oxide, prodrug, isotopic variant (e.g., deuterated analog), PROTAC, polymorph, or crystal of said compound, or any combination thereof. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the cancer is multiple myeloma. In some embodiments, the cancer is leukemia. In some embodiments, the cancer is lymphoma. In some embodiments, the cancer is breast cancer. In some embodiments, the cancer is prostate cancer. In some embodiments, the cancer is ovarian cancer. In some embodiments, the cancer is cervical cancer. In some embodiments, the cancer is uterine cancer. In some embodiments, the cancer is colon carcinoma. In some embodiments, the cancer is lung cancer. In some embodiments, the cancer is renal cancer. In some embodiments, the cancer is melanoma. In some embodiments, the cancer is CNS. In some embodiments, the cancer is bone cancer. In some embodiments, the cancer is CNS. In some embodiments, the cancer is colorectal cancer.

In various embodiments, this invention provides methods for increasing the survival of a subject suffering from metastatic cancer comprising the step of administering to said subject a compound of this invention and/or an isomer, metabolite, pharmaceutically acceptable salt, pharmaceutical product, tautomer, hydrate, N-oxide, prodrug, isotopic variant (e.g., deuterated analog), PROTAC, polymorph, or crystal of said compound, or any combination thereof. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the cancer is multiple myeloma. In some embodiments, the cancer is leukemia. In some embodiments, the cancer is lymphoma. In some embodiments, the cancer is breast cancer. In some embodiments, the cancer is prostate cancer. In some embodiments, the cancer is ovarian cancer. In some embodiments, the cancer is cervical cancer. In some embodiments, the cancer is uterine cancer. In some embodiments, the cancer is colon carcinoma. In some embodiments, the cancer is lung cancer. In some embodiments, the cancer is renal cancer. In some embodiments, the cancer is melanoma. In some embodiments, the cancer is CNS. In some embodiments, the cancer is bone cancer. In some embodiments, the cancer is colorectal cancer.

In various embodiments, this invention provides methods for treating, suppressing, reducing the severity, reducing the risk, or inhibiting advanced cancer comprising the step of administering to said subject a compound of this invention and/or an isomer, metabolite, pharmaceutically acceptable salt, pharmaceutical product, tautomer, hydrate, N-oxide, prodrug, isotopic variant (e.g., deuterated analog), PROTAC, polymorph, or crystal of said compound, or any combination thereof. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the cancer is multiple myeloma. In some embodiments, the cancer is leukemia. In some embodiments, the cancer is lymphoma. In some embodiments, the cancer is breast cancer. In some embodiments, the cancer is prostate cancer. In some embodiments, the cancer is ovarian cancer. In some embodiments, the cancer is cervical cancer. In some embodiments, the cancer is uterine cancer. In some embodiments, the cancer is colon carcinoma. In some embodiments, the cancer is lung cancer. In some embodiments, the cancer is renal cancer. In some embodiments, the cancer is melanoma. In some embodiments, the cancer is CNS. In some embodiments, the cancer is bone cancer. In some embodiments, the cancer is colorectal cancer.

In various embodiments, this invention provides methods for increasing the survival of a subject suffering from advanced cancer comprising the step of administering to said subject a compound of this invention and/or an isomer, metabolite, pharmaceutically acceptable salt, pharmaceutical product, tautomer, hydrate, N-oxide, prodrug, isotopic variant (e.g., deuterated analog), PROTAC, polymorph, or crystal of said compound, or any combination thereof. In some embodiments, the compound is a protein degradation inhibitor. In some embodiments, the compound is a UPS inhibitor. In some embodiments, the compound is an autophagy modulator. In some embodiments, the compound is a UPR inducer. In some embodiments, the cancer is multiple myeloma. In some embodiments, the cancer is leukemia. In some embodiments, the cancer is lymphoma. In some embodiments, the cancer is breast cancer. In some embodiments, the cancer is prostate cancer. In some embodiments, the cancer is ovarian cancer. In some embodiments, the cancer is cervical cancer. In some embodiments, the cancer is uterine cancer. In some embodiments, the cancer is colon carcinoma. In some embodiments, the cancer is lung cancer. In some embodiments, the cancer is renal cancer. In some embodiments, the cancer is melanoma. In some embodiments, the cancer is CNS. In some embodiments, the cancer is bone cancer. In some embodiments, the cancer is colorectal cancer.

The compounds of the present invention are useful in the treatment, reducing the severity, reducing the risk, or inhibition of cancer, metastatic cancer, advanced cancer, drug resistant cancer, and various forms of cancer. In a preferred embodiment the cancer is multiple myeloma, leukemia, lymphoma, breast cancer, ovarian cancer, cervical cancer, uterine cancer, colon cancer, lung cancer, renal cancer, prostate cancer, melanoma, CNS, colorectal cancer and bone cancer; each represents a separate embodiment according to this invention. Based upon their believed mode of action, it is believed that other forms of cancer will likewise be treatable or preventable upon administration of the compounds or compositions of the present invention to a patient. Preferred compounds of the present invention are selectively disruptive to cancer cells, causing ablation of cancer cells but preferably not normal cells. Significantly, harm to normal cells is minimized because the cancer cells are susceptible to disruption at much lower concentrations of the compounds of the present invention.

In various embodiments, other types of cancers that may be treatable with the protein degradation inhibitors according to this invention include: multiple myeloma, leukemia, lymphoma, breast cancer, ovarian cancer, cervical cancer, uterine cancer, colon cancer, colorectal cancer, lung cancer, renal cancer, prostate cancer, melanoma, central nervous system (CNS) cancer, bone cancer, adrenocortical carcinoma, anal cancer, bladder cancer, brain tumor, brain stem tumor, glioma, cerebellar astrocytoma, cerebral astrocytoma, ependymoma, medulloblastoma, supratentorial primitive neuroectodermal, pineal tumors, hypothalamic glioma, carcinoid tumor, carcinoma, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, non-small cell lung cancer, small cell, lymphoma, AIDS-related lymphoma, central nervous system (primary), cutaneous T-cell lymphoma, Hodgkin's disease, non-Hodgkin's disease, malignant mesothelioma, Merkel cell carcinoma, metasatic squamous carcinoma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, rhabdomyosarcoma, rectal cancer, renal cell cancer, salivary gland cancer, Sezary syndrome, skin cancer, skin cancer, Kaposi's sarcoma, small intestine cancer, soft tissue sarcoma, testicular cancer, thymoma, thyroid cancer, urethral cancer, sarcoma, unusual cancer of childhood, vaginal cancer, vulvar cancer, Wilms' tumor, hepatocellular cancer, hematological cancer or any combination thereof. In some embodiments the cancer is invasive. In some embodiments the cancer is metastatic cancer. In some embodiments the cancer is advanced cancer. In some embodiments the cancer is drug resistant cancer.

In various embodiments “metastatic cancer” refers to a cancer that spread (metastasized) from its original site to another area of the body. Virtually all cancers have the potential to spread. Whether metastases develop depends on the complex interaction of many tumor cell factors, including the type of cancer, the degree of maturity (differentiation) of the tumor cells, the location and how long the cancer has been present, as well as other incompletely understood factors. Metastases spread in three ways—by local extension from the tumor to the surrounding tissues, through the bloodstream to distant sites or through the lymphatic system to neighboring or distant lymph nodes. Each kind of cancer may have a typical route of spread. The tumor is called by the primary site (ex. breast cancer that has spread to the brain is called metastatic breast cancer to the brain).

In various embodiments “drug-resistant cancer” refers to cancer cells that acquire resistance to chemotherapy. Cancer cells can acquire resistance to chemotherapy by a range of mechanisms, including the mutation or overexpression of the drug target, inactivation of the drug, or elimination of the drug from the cell. Tumors that recur after an initial response to chemotherapy may be resistant to multiple drugs (they are multidrug resistant). In the conventional view of drug resistance, one or several cells in the tumor population acquire genetic changes that confer drug resistance. Accordingly, the reasons for drug resistance, inter alia, are: a) some of the cells that are not killed by the chemotherapy mutate (change) and become resistant to the drug. Once they multiply, there may be more resistant cells than cells that are sensitive to the chemotherapy; b) Gene amplification. A cancer cell may produce hundreds of copies of a particular gene. This gene triggers an overproduction of protein that renders the anticancer drug ineffective; c) cancer cells may pump the drug out of the cell as fast as it is going in using a molecule called p-glycoprotein; d) cancer cells may stop taking in the drugs because the protein that transports the drug across the cell wall stops working; e) the cancer cells may learn how to repair the DNA breaks caused by some anti-cancer drugs; f) cancer cells may develop a mechanism that inactivates the drug. One major contributor to multidrug resistance is overexpression of P-glycoprotein (P-gp). This protein is a clinically important transporter protein belonging to the ATP-binding cassette family of cell membrane transporters. It can pump substrates including anticancer drugs out of tumor cells through an ATP-dependent mechanism; g) Cells and tumors with activating RAS mutations are relatively resistant to most anti-cancer agents. Thus, the resistance to anticancer agents used in chemotherapy is the main cause of treatment failure in malignant disorders, provoking tumors to become resistant. Drug resistance is the major cause of cancer chemotherapy failure.

In various embodiments “resistant cancer” refers to drug-resistant cancer as described herein above. In some embodiments “resistant cancer” refers to cancer cells that acquire resistance to any treatment such as chemotherapy, radiotherapy or biological therapy.

In various embodiments, this invention is directed to treating, suppressing, reducing the severity, reducing the risk, or inhibiting cancer in a subject, wherein the subject has been previously treated with chemotherapy, radiotherapy or biological therapy.

In various embodiments “Chemotherapy” refers to chemical treatment for cancer such as drugs that kill cancer cells directly. Such drugs are referred as “anti-cancer” drugs or “antineoplastics.” Today's therapy uses more than 100 drugs to treat cancer. To cure a specific cancer. Chemotherapy is used to control tumor growth when cure is not possible; to shrink tumors before surgery or radiation therapy; to relieve symptoms (such as pain); and to destroy microscopic cancer cells that may be present after the known tumor is removed by surgery (called adjuvant therapy). Adjuvant therapy is given to prevent a possible cancer reoccurrence.

In various embodiments, “Radiotherapy” (also referred herein as “Radiation therapy”) refers to high energy x-rays and similar rays (such as electrons) to treat disease. Many people with cancer will have radiotherapy as part of their treatment. This can be given either as external radiotherapy from outside the body using x-rays or from within the body as internal radiotherapy. Radiotherapy works by destroying the cancer cells in the treated area. Although normal cells can also be damaged by the radiotherapy, they can usually repair themselves. Radiotherapy treatment can cure some cancers and can also reduce the chance of a cancer coming back after surgery. It may be used to reduce cancer symptoms.

In various embodiments “Biological therapy” refers to substances that occur naturally in the body to destroy cancer cells. There are several types of treatment including: monoclonal antibodies, cancer growth inhibitors, vaccines and gene therapy. Biological therapy is also known as immunotherapy.

When the compounds or pharmaceutical compositions of the present invention are administered to treat, suppress, reduce the severity, reduce the risk, or inhibit a cancerous condition, the pharmaceutical composition can also contain, or can be administered in conjunction with, other therapeutic agents or treatment regimen presently known or hereafter developed for the treatment of various types of cancer. Examples of other therapeutic agents or treatment regimen include, without limitation, radiation therapy, immunotherapy, chemotherapy, surgical intervention, and combinations thereof.

In various embodiments, the compound according to this invention, is administered in combination with an anti-cancer therapy. Examples of such therapies include but are not limited to: chemotherapy, immunotherapy, radiotherapy, biological therapy, surgical intervention, and combinations thereof.

In various embodiments, the compound is administered in combination with an anti-cancer agent by administering the compounds as herein described, alone or in combination with other agents.

In various embodiments, the composition for cancer treatment of the present invention can be used together with existing chemotherapy drugs or be made as a mixture with them. Such a chemotherapy drug includes, for example, alkylating agents, nitrosourea agents, antimetabolites, antitumor antibiotics, alkaloids derived from plant, topoisomerase inhibitors, hormone therapy medicines, hormone antagonists, aromatase inhibitors, P-glycoprotein inhibitors, platinum complex derivatives, other immunotherapeutic drugs, and other anticancer agents. Further, they can be used together with hypoleukocytosis (neutrophil) medicines that are cancer treatment adjuvant, thrombopenia medicines, antiemetic drugs, and cancer pain medicines for patient's QOL recovery or be made as a mixture with them.

In various embodiments, this invention is directed to a method of destroying a cancerous cell comprising providing a compound of this invention and contacting the cancerous cell with the compound under conditions effective to destroy the contacted cancerous cell. According to various 9?embodiments of destroying the cancerous cells, the cells to be destroyed can be located either in vivo or ex vivo (i.e., in culture).

A still further aspect of the present invention relates to a method of treating or preventing a cancerous condition that includes providing a compound of the present invention and then administering an effective amount of the compound to a patient in a manner effective to treat or prevent a cancerous condition.

According to one embodiment, the patient to be treated is characterized by the presence of a precancerous condition, and the administering of the compound is effective to prevent development of the precancerous condition into the cancerous condition. This can occur by destroying the precancerous cell prior to or concurrent with its further development into a cancerous state.

According to other embodiments, the patient to be treated is characterized by the presence of a cancerous condition, and the administering of the compound is effective either to cause regression of the cancerous condition or to inhibit growth of the cancerous condition, i.e., stopping its growth altogether or reducing its rate of growth. This preferably occurs by destroying cancer cells, regardless of their location in the patient body. That is, whether the cancer cells are located at a primary tumor site or whether the cancer cells have metastasized and created secondary tumors within the patient body.

In some embodiments, the present invention is a method for reducing growth of at least one tumor in a subject comprising: obtaining a compound according to this invention and administering a therapeutically effective amount of a compound according to this invention for a sufficient period of time so as to result in reducing growth of the at least one tumor in the subject, compared to an untreated tumor, e.g. by 30 to 70 percent.

In some embodiments, the sufficient period of time is from 1 to 20 weeks. In some embodiments, the sufficient period of time is from 2 to 20 weeks. In some embodiments, the sufficient period of time is from 3 to 20 weeks. In some embodiments, the sufficient period of time is from 4 to 20 weeks. In some embodiments, the sufficient period of time is from 5 to 20 weeks. In some embodiments, the sufficient period of time is from 6 to 20 weeks. In some embodiments, the sufficient period of time is from 8 to 20 weeks. In some embodiments, the sufficient period of time is from 10 to 20 weeks. In some embodiments, the sufficient period of time is from 12 to 20 weeks. In some embodiments, the sufficient period of time is from 14 to 20 weeks. In some embodiments, the sufficient period of time is from 16 to 20 weeks. In some embodiments, the sufficient period of time is from 18 to 20 weeks.

In some embodiments, the sufficient period of time is from 1 to 18 weeks. In some embodiments, the sufficient period of time is from 1 to 16 weeks. In some embodiments, the sufficient period of time is from 1 to 14 weeks. In some embodiments, the sufficient period of time is from 1 to 12 weeks. In some embodiments, the sufficient period of time is from 1 to 10 weeks. In some embodiments, the sufficient period of time is from 1 to 8 weeks. In some embodiments, the sufficient period of time is from 1 to 6 weeks. In some embodiments, the sufficient period of time is from 1 to 4 weeks. In some embodiments, the sufficient period of time is from 1 to 2 weeks. In some embodiments, the sufficient period of time is from 2 to 4 weeks.

In some embodiments, the sufficient period of time is from 2 to 18 weeks. In some embodiments, the sufficient period of time is from 4 to 16 weeks. In some embodiments, the sufficient period of time is from 6 to 14 weeks. In some embodiments, the sufficient period of time is from 8 to 12 weeks.

In some embodiments, the therapeutically effective amount of a compound according to this invention, pharmaceutically acceptable salts or solvates thereof, is equivalent to an animal dose ranging from 0.1 mg/kg to 50 mg/kg.

In some embodiments, the therapeutically effective amount of a compound according to this invention, pharmaceutically acceptable salts or solvates thereof, ranges from 0.08 mg/kg to 4 mg/kg in humans. In some embodiments, the therapeutically effective amount of a compound according to this invention ranges from 0.1 mg/kg to 1 mg/kg in humans. In some embodiments, the therapeutically effective amount of a compound according to this invention ranges from 0.1 mg/kg to 10 mg/kg in humans.

In some embodiments, a compound according to this invention is administered daily, every other day, 5 times a week, 4 times a week, 3 times a week, twice a week, or once a week.

It should be understood that the regimen of administration can affect the effective amount. It should also be understood that a specific dosage and treatment regimen for any particular patient will depend upon a variety of factors, including the age, body weight, general health, sex, and diet of the patient, time of administration, drug combinations, the judgment of the treating physician, and the severity of the particular disease being treated.

In some embodiments, the therapeutically effective amount of a compound according to this invention is equivalent to an animal dose ranging from 0.1 mg/kg to 50 mg/kg

In some embodiments, the subject is a mammal. In some embodiments, the subject is a human. In some embodiments, the subject is a domestic animal, e.g., but not limited to, a dog, a cat, a rabbit, etc.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, throughout the specification, the meaning of “a,” “an,” and “the” include plural references. The meaning of “in” includes “in” and “on.”

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

### EXAMPLES

### General Methods

Preparative HPLC was performed on a Gilson system equipped with a UV detector using an XBridge Prep C-18 5 μm OBD, 19×50 mm column. Analytical HPLC-MS was performed using an Agilent 1100 series Liquid Chromatograph/Mass Selective Detector (MSD) (Single Quadropole) equipped with an electrospray interface and a UV diode array detector. Anal-yses were performed by two methods using either an ACE 3 C8 (3.0×50 mm) column with a gradient of acetonitrile in 0.1% aqueous TFA over 3 min and a flow of 1 mL/min, or an XBridge C18 (3.0×50 mm) column with a gradient of acetonitrile in 10 mM ammonium bicarbonate over 3 min and a flow of 1 mL/min. 1H-NMR spectra were recorded on a Bruker 400 MHz instrument at 25° C. The compounds have been named using the software MarvinSketch. In addition, the commercial names or trivial names were used for the commercial starting materials and reagents. All chromatography purifications were performed on silica gel (Sigma Aldrich) high-purity grade, pore size 60A, particle size 40-63 um; TLC silica gel 60F254 (Merck).

### Example 1

### Synthesis of Compound B1

The reaction below shows the synthesis of Compound B1:

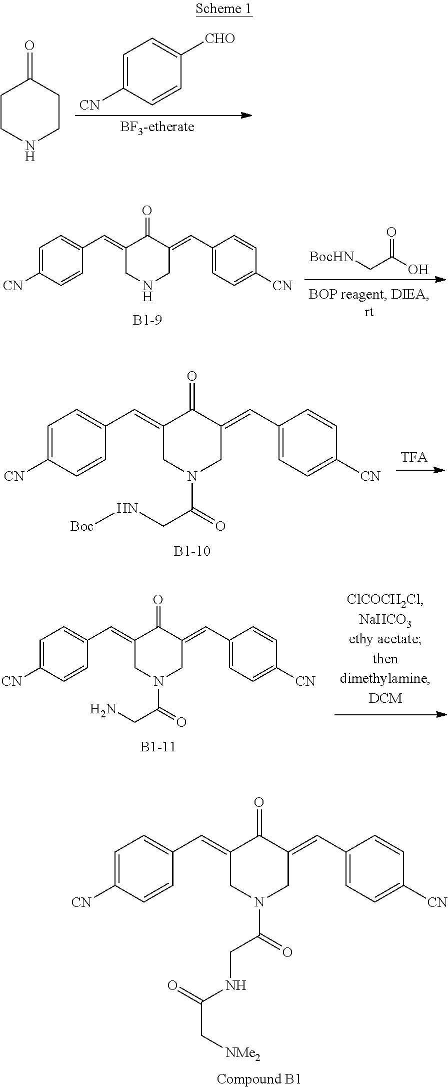

### Experimental Procedure

### Synthesis of Compound B1-9 (Scheme 1)

Procedure A: In a 250 milliliters (mL) flask, 2.0 grams (g) of 4-piperidone monohydrate hydrochloride was cooled in an ice water bath. Boron trifluoride diethyl etherate (22 mL) was added and to the stirred solution was added 3.4 g (2 equivalents) of 4-formyl benzonitrile. The reaction was warmed to ambient temperature and stirred for 24 hours (h). Saturated NaHCO₃ was poured into the reaction mixture and the resulting yellow solid was collected by filtration, washed with water then ethyl acetate. Upon drying 1.92 g of yellow solid compound B1-9 was obtained (45% yield).

Procedure B: 4-Piperidinone monohydrate hydrochloride (500 mg, 3.3 mmol) was placed in a 25 ml round bottom flask and cooled to 0° C. Boron trifluoride etherate (5 mL) was added dropwise, then aldehyde (854 mg, 6.6 mmol) was added to the reaction mixture in one portion. The reaction was stirred overnight at room temperature under nitrogen atmosphere. The reaction was carefully quenched with a saturated solution of NaHCO₃. A Yellow solid precipitated out was filtered under reduced pressure, washed with water and EtOH to give B1-9 (734 mg, 2.25 mmol, 71%) (Scheme 16). ¹H NMR (400 MHz, DMSO-d₆) δ 7.92 (d, J=8.3 Hz, 4H), 7.67 (d, J=8.3 Hz, 4H), 7.61 (s, 2H), 3.99 (s, 4H), 2.85 (s, 1H).

Procedure C: In a 50 ml flask 4-piperidinone monohydrate hydrochloride (1.434 gr, 1 eq) was dissolved in acetic acid (20 ml). Then 4-formylbenzonitrile (2.422 gr, 2 eq) was added followed by slowly addition of 1 ml sulfuric acid. The clear solution was stirred at r.t. overnight. Pre-cipitation was viewed, the product was observed by LC-MS and 7 ml of water were added. The product was separated by centrifugation and washed with methanol (16 ml×2) and diethyl ether (12 ml) with centrifugation in between. The yellow solid was dried under high vacuum overnight to obtain B1-9 (1.01 g, 33% yield) (Scheme 27). HPLC purity: 97%; MS (ESI+) m/z 326.0 [M+H]+.

### Synthesis of Compound B1-10 (Scheme 1)

The aldol product compound B1-9 (1 g, 3.1 mmol) and Boc-Gly-OH (0.54 g, 1 equivalent) were suspended in 15 mL dimethylformamide (DMF). (Benzotriazol-1-yloxy)tris(dimethylamino)phosphonium hexafluorophosphate (BOP reagent) (1.33 g, 1 equivalent) was added, followed by N,N-Diisopropylethylamine (DIPEA) (1.6 mL, 3 equivalents). The reaction was stirred at room temperature. After 1 h, the reaction mixture became a clear solution. Upon completion (as determined by thin layer chromatography (TLC)), the reaction mixture was poured into water. The resulting solid was collected by filtration, washed with water, ethyl acetate and methanol. Upon drying 1.42 g of compound B1-10 was obtained.

### Synthesis of Compound B1 (Scheme 1)

Compound B1-10 (390 mg) stirred in 1.5 mL of 2,2,2-trifluoroacetic acid (TFA) at ambient temperature for 2 hours. The reaction mixture was concentrated under reduced pressure and the residue suspended in 5 mL ethyl acetate. Saturated sodium bicarbonate solution (5 mL) was added, followed by chloroacetyl chloride (5 equivalents). The reaction mixture was stirred vigorously for 2 hours and the resulting solid (compound 11) collected by filtration, washed with water and ethyl acetate. Upon drying the solid compound 11 was dissolved in 5 mL dichloromethane (DCM) and 1 equivalent of dimethylamine (2.0 M solution in tetrahydrofuran (THF)) was added. Upon stirring 2 hours at ambient temperature the reaction mixture was concentrated and the residue purified by column chromatography to give 135 mg of final product Compound B1. The results were: ¹H NMR (CDCl3, 400 MHz): δ 2.28 (s, 6H), 2.94 (s, 2H), 3.98 (d, 2H, J=4 Hz), 4.73 (s, 2H), 4.89 (s, 2H), 7.55 (m, 5H), 7.84 (M, 5H); HPLC purity: 95%; MS (ESI+) m/z 468.19 [M+H]+.

### Synthesis of Salts of Compound B1

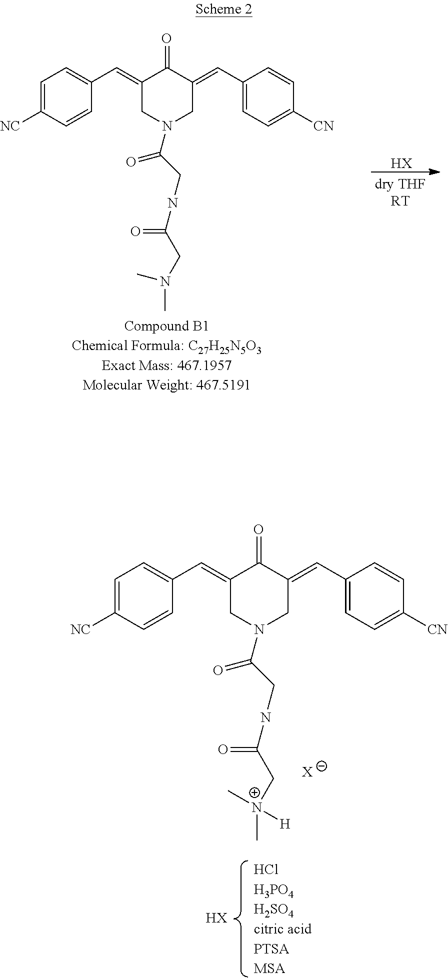

Compound B1 (50 mg, 0.1 mmol) was totally dissolved in dry THF (about 10 mL), then the appropriate acid (0.15 mmol) was added slowly to the solution. The reaction mixture was stirred at room temperature for at least 2 hr. Upon completion (as determined by TLC), the resulting solid was collected.

### Example 2

### Synthesis of Compound C1

The synthesis of partially reduced Compound C1 is shown below:

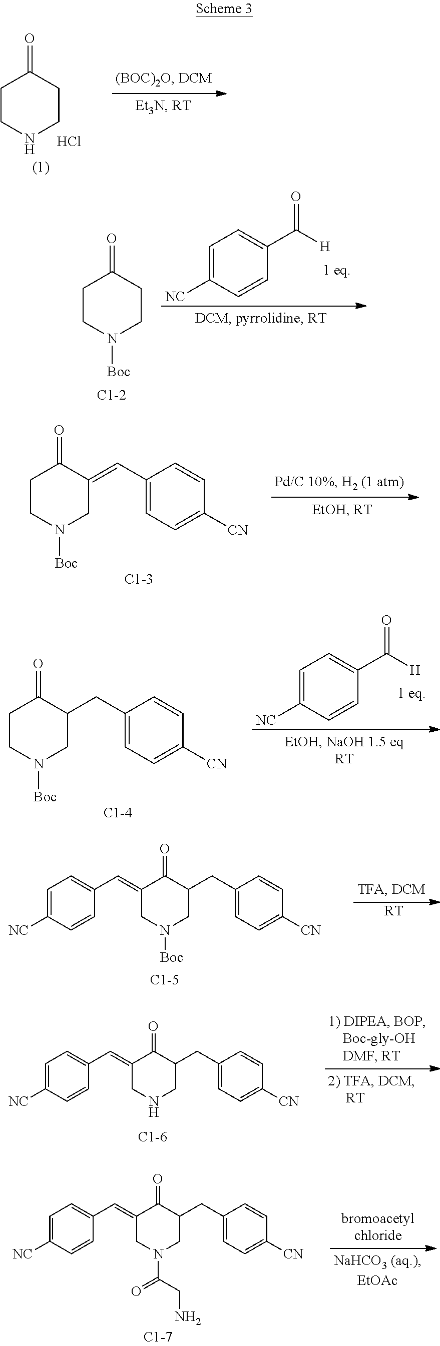

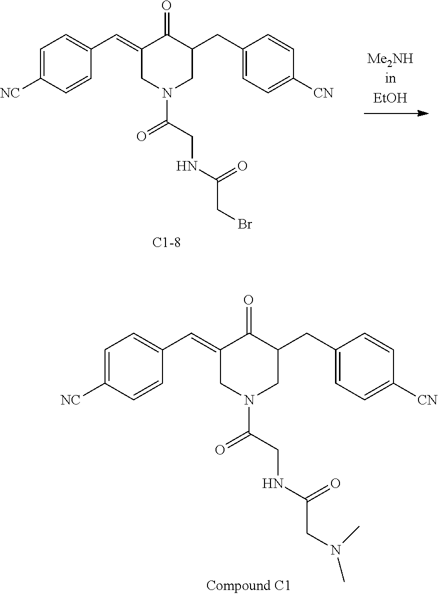 [SMILES: N#Cc1ccc(/C=C2\CN(C(=O)CNC(=O)CBr)CC(Cc3ccc(C#N)cc3)C2=O)cc1 ; CN(C)CC(=O)NCC(=O)N1C/C(=C\c2ccc(C#N)cc2)C(=O)C(Cc2ccc(C#N)cc2)C1]

### Experimental Procedure

### Synthesis of Compound (C1-2) (Scheme 3)

In a 250 ml flask, 4-piperidone monohydrate hydrochloride (4 g) [compound (1)] and triethyl amine (2 eq.) was dissolved in DCM (30 ml). Then di-tert-butyl dicarbonate (Boc anhydride) (1eq.) was added. The reaction mixture was stirred for overnight at room temperature (RT). The reaction mixture was poured into water, extracted with dichloromethane (DCM), dried over sodium sulfate, filtered and concentrated. Upon drying 5.3 gr of white solid was obtained (88% yield).

¹H-NMR (CDCl₃): 1.43 (s, 9H), 2.37 (t, 4H), 3.65 (t, 4H).

### Synthesis of Compound (3) (Scheme 3)

BOC-protected 4-piperidone C₁₋₂ (1.5 g) and pyrrolidine (0.6 ml, 1 eq.) was dissolved in DCM (20 ml), and then 4-cyanobenzaldeyde (1.eq, 1 gr) was added. The reaction mixture was stirred for overnight under nitrogen at room temperature. The solvent was evaporated to dryness and the crude was purified by column chromatography (5%-40% ethyl acetate—hexane) to give compound C₁₋₃ as a white solid (1.5 g, 63%). The product was confirmed by GC-MS.

### Synthesis of Compound (C1-4) (Scheme 3)

Compound C1-3 (0.5 g) was dissolved in ethanol and Pd/C (10% w/w) was added to the solution. The mixture was stirred under hydrogen atmosphere for 2 hr. After completion of the reaction; the mixture was filtered through Celite pad and filtrate was evaporated. The product was confirmed by GC-MS. (quantitative yield).

### Synthesis of Compound (C1-5) (Scheme 3)

To a solution of compound C₁₋₄ (0.5 g) and 4-cyanobenzaldehyde (1 eq.) in EtOH was added the solution of NaOH (1.50 eq) in EtOH (5.00 mL). The mixture was stirred at RT for 3 hr. The reaction was followed by TLC and HPLC. After completion, water was added and extracted with ethyl acetate. The organic phase was washed with brine and dried over Na₂SO₄, filtered and evaporated and after silica gel column chromatography, the product was obtained as a yellow solid (70% yield).

### Synthesis of Compound (C1-6) (Scheme 3)

To the solution of compound C₁₋₅ (0.45 g) in DCM (15 ml) was added TFA (1 ml) and the reaction mixture was stirred at RT. After completion by HPLC the solvent was evaporated, and the mixture was used as such in the next step with further purification.

### Synthesis of Compound (C1-7) (Scheme 3)

Compound C1-6 (0.4 g) and boc-gly-OH (1 eq.) were dissolved in DMF (10 ml). BOP reagent (1 eq.) was added, followed by DIPEA (4eq.). The reaction mixture was stirred at room temperature for 1 hr. the mixture poured into water and extracted with ethyl acetate. The organic phase dried over sodium sulfate, filtered and evaporated to dryness. The crude was dissolved in DCM (15 ml) and TFA (1 ml) was then added and the reaction mixture stirred at RT. Upon completion by HPLC, the solvent was evaporated to make compound C₁₋₇.

### Synthesis of Compound (C1-8) and Compound C1 (Scheme 3)

The crude mixture from the previous step was dissolved in ethyl acetate (20 ml). Saturated sodium bicarbonate solution was added, followed by the addition of bromoacetyl chloride (3eq.). The reaction mixture was stirred at RT and the reaction progress was determined using HPLC. Upon completion, the organic phase was separated and dried over sodium sulfate, filtered and evaporated. After drying, the crude compound 8 was dissolved in DCM and 3 eq. of dimethylamine in ethanol was added. After 2 hr (as determined by HPLC), the solvent was evaporated and the crude mixture was purified by column chromatography using 5% MeOH/2% Et₃N-DCM as the eluent to give partially reduced Compound C1 (100 mg).

The structure of Compound C1 was confirmed by LC-MS and ¹HNMR. The concentration/purity of major isomer at 8.67 minute in HPLC at 225 nM, 254 nM, 270 nM, and 285 nM wavelengths showed from 91.5% to 97.2%. The intensity of the minor isomers were from 0.4% to 3.1%.

### Example 3

### Synthesis of Compound AA and Salts Thereof

The synthesis of Compound AA is shown below:

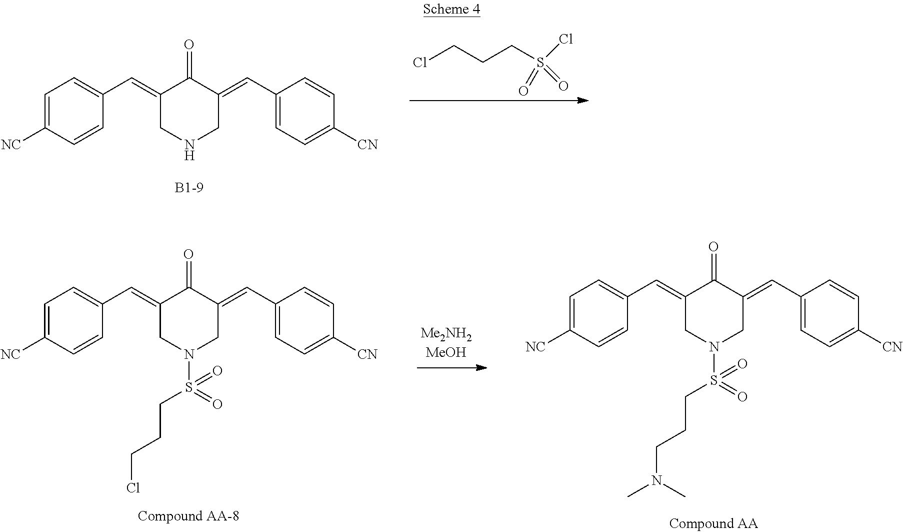 [SMILES: N#Cc1ccc(/C=C2\CNC/C(=C\c3ccc(C#N)cc3)C2=O)cc1 ; O=S(=O)(Cl)CCCCl ; N#Cc1ccc(/C=C2\CN(S(=O)(=O)CCCCl)C/C(=C\c3ccc(C#N)cc3)C2=O)cc1 ; CN(C)CCCS(=O)(=O)N1C/C(=C\c2ccc(C#N)cc2)C(=O)/C(=C/c2ccc(C#N)cc2)C1]

### Synthesis of Compound (AA-8) (Scheme 4)

Compound B1-9 (3 gr, 9.2 mmol) and triethylamine (TEA) (37 mmol, 2.7 ml) were stirred in 100 mL dichloromethane (DCM) under nitrogen atmosphere. The reaction was cooled down to 0° C., then, sulfonyl chloride (3.56 ml, 13.8 mmol) was added dropwise and the reaction stirred for 3 hours at r.t. Saturated NaHCO₃(30 ml) was added to the reaction mixture and extracted with 30 ml DCM three times. The combined organic phases were dried with anhydrous sodium sulfate and evaporated under reduced pressure. The crude was purified with silica gel chromatography (20% ethyl acetate (EtOAc) in hexanes). Yellow solid Compound AA-8 (2.85 gr, 67% yield) was isolated.

### Synthesis of Compound AA (Scheme 4)

Compound AA-8 (424 mg, 0.9 mmol) was dissolved in 10 ml, 5.6M dimethyl amine solution in ethanol. Catalytic amount of sodium iodide was added and the reaction stirred at room temperature for 4 days. The reaction mixture quenched with 10 ml NaHCO₃ saturated solution and extracted with 10 ml DCM three times. The combined organic phase were dried with sodium sulfate and evaporated under reduced pressure. The crude was purified with silica gel chromatography (A=1% TEA in methanol (MeOH); B=DCM mixture, gradient of A in B up to 20%). 223 mg of yellow solid was obtained (0.47 mmol, 52%). Thin layer chromatography (TLC) (3% MeOH in DCM, 1 drop of TEA): retention factor (Rf)=0.16.

¹H NMR (600 MHz, DMSO) δ 7.96 (d, J=7.9 Hz, 4H), 7.77 (s, 2H), 7.73 (d, J=7.9 Hz, 4H), 4.65 (s, 4H), 3.18 (t, 2H), 2.28 (t, J=6.4 Hz, 6H), 1.73 (m, 2H); (high performance liquid chromatography (HPLC) purity 95%.

### Formation of Compound AA's Salts (Scheme 5)

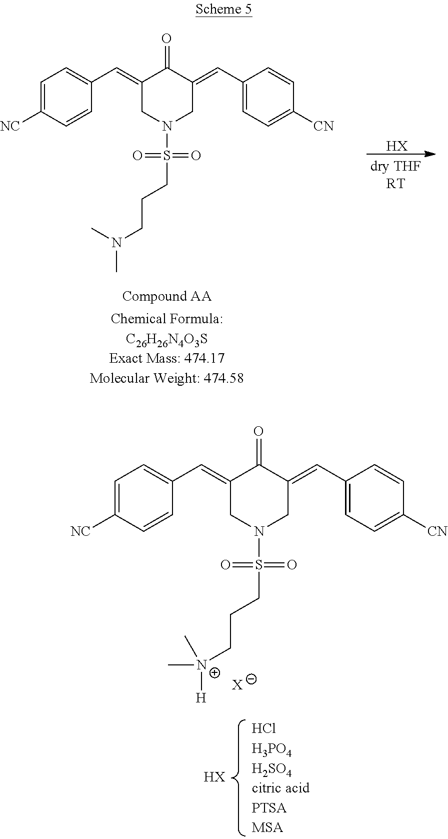

Compound AA (50 mg, 0.1 mmol) was completely dissolved in dry THF (ca. 10 ml), then the appropriate acid (0.15 mmol) was added slowly to the solution. The reaction mixture was stirred at room temperature for at least 2 hours. Upon completion (as determined by thin layer chromatography (TLC)), the resulting solid was collected.

### Example 4

### Synthesis of Compound E1

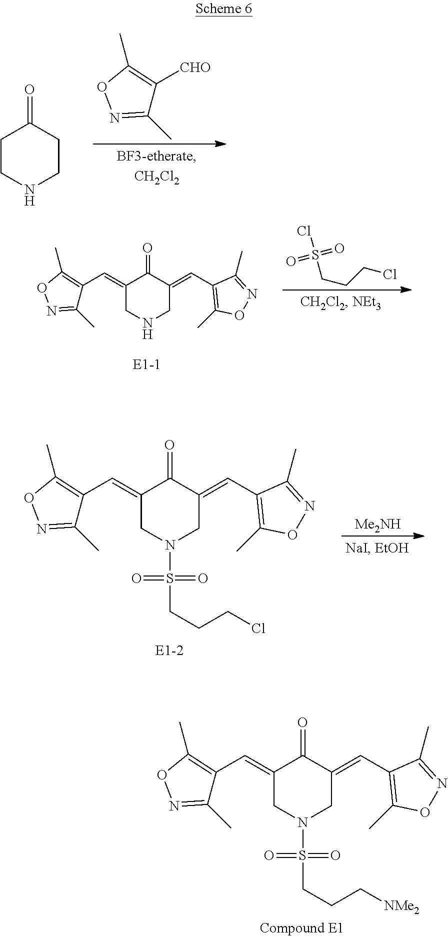

### Synthesis of Intermediate (E1-1) (Scheme 7)

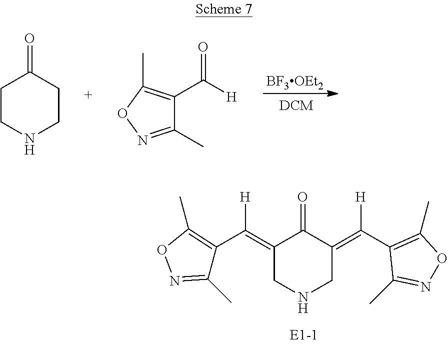

4-Piperidinone (0.92 gr, 6 mmol) was placed in a 25 ml round bottom flask and cooled to 0° C. Boron trifluoride (10 mL) was added dropwise followed by the aldehyde (1.5 gr, 12 mmol) in one portion. The reaction mixture was stirred overnight at room temperature under nitrogen atmosphere. The reaction was carefully quenched with a saturated solution of NaHCO₃. The solid precipitated out from the solution was filtered under reduced pressure, washed with water and EtOH to give the intermediate E1-1 (Scheme 7) as a yellow solid (1.2 gr, 3.8 mmol, 63%).

### Synthesis of Intermediate (E1-2) (Scheme 8)

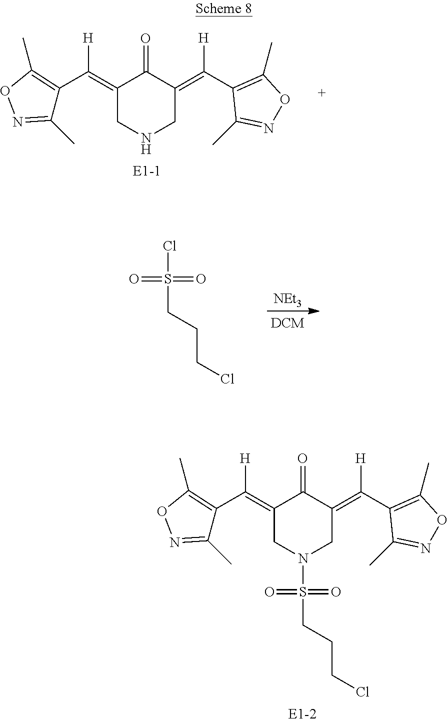 [SMILES: Cc1noc(C)c1/C=C1\CNC/C(=C\c2c(C)noc2C)C1=O ; O=S(=O)(Cl)CCCCl ; Cc1noc(C)c1/C=C1\CN(S(=O)(=O)CCCCl)C/C(=C\c2c(C)noc2C)C1=O ; "NEt3"]

Intermediate E1-1 (0.5 gr, 1.6 mmol) (Scheme 8) and triethylamine (6.7 mmol, 0.7 ml) were stirred in dichloromethane (100 mL) under nitrogen atmosphere. The reaction was cooled to 0° C., and chloropropylsulfonyl chloride (3.56 ml, 13.8 mmol) was added dropwise. The reaction was stirred for 3 h at room temperature. Saturated NaHCO₃ (30 ml) was added to the reaction mixture and extracted with DCM (30 ml×3). The combined organic phases were dried with anhydrous sodium sulfate and evaporated under reduced pressure. The crude was purified with silica gel column chromatography (20% EtOAc in hexanes) provided a yellow solid (0.43 gr, 59% yield).

### Synthesis of Compound E1 (Scheme 9)

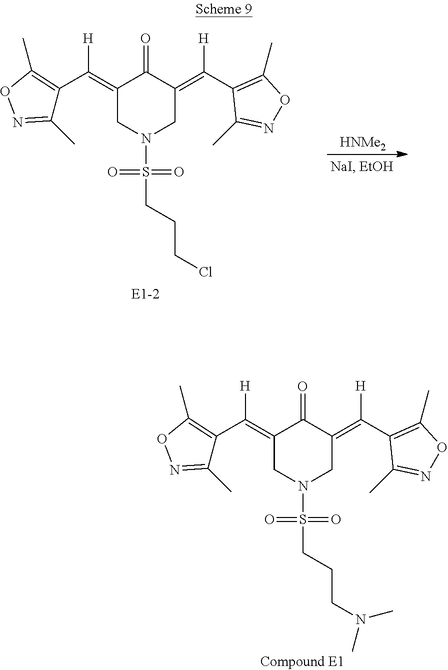 [SMILES: Cc1noc(C)c1/C=C1\CN(S(=O)(=O)CCCCl)C/C(=C\c2c(C)noc2C)C1=O ; Cc1noc(C)c1/C=C1\CN(S(=O)(=O)CCCN(C)C)C/C(=C\c2c(C)noc2C)C1=O ; "HNMe2"]

Intermediate E1-2 (300 mg, 0.66 mmol) (Scheme 9) was dissolved in 5.6M dimethyl amine solution in ethanol (10 mL). Catalytic amount of sodium iodide was added and the reaction mixture was stirred at room temperature for 4 days. The reaction mixture was quenched with 20 ml saturated NaHCO₃ solution and extracted with DCM (20 ml×3). The combined organic extract was dried with anhydrous sodium sulfate and evaporated under reduced pressure. The crude was purified with silica gel column chromatography (A=1% TEA in MeOH; B=DCM mixture, gradient of A in B up to 20%) provided a yellow solid (170 mg, 0.37 mmol, 56% yield). HPLC purity-96%.

¹H NMR (400 MHz, DMSO-D₆) δ 7.38 (s, 2H), 4.23 (s, 4H), 3.16-3.02 (m, 2H), 2.46-2.37 (m, J=7.6 Hz, 2H), 2.36 (s, 6H), 2.24 (bs, 6H), 2.20 (bs, 6H), 1.81-1.70 (m, 2H).

### Example 5

### Synthesis of Compound F1

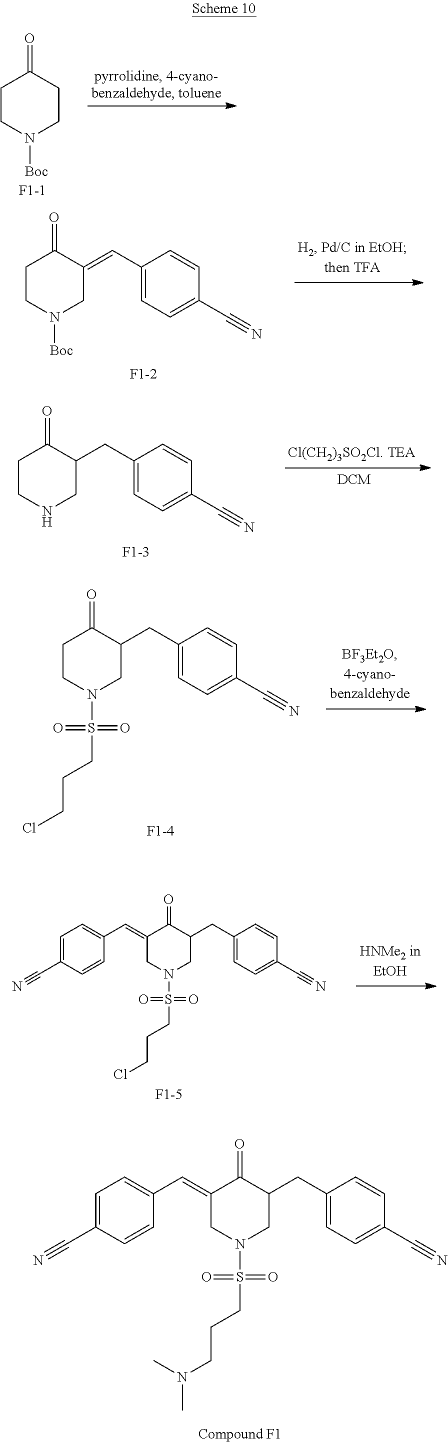 [SMILES: CC(C)(C)OC(=O)N1CCC(=O)CC1 ; CC(C)(C)OC(=O)N1CCC(=O)/C(=C/c2ccc(C#N)cc2)C1 ; N#Cc1ccc(CC2CNCCC2=O)cc1 ; N#Cc1ccc(CC2CN(S(=O)(=O)CCCCl)CCC2=O)cc1 ; N#Cc1ccc(/C=C2\CN(S(=O)(=O)CCCCl)CC(Cc3ccc(C#N)cc3)C2=O)cc1 ; CN(C)CCCS(=O)(=O)N1C/C(=C\c2ccc(C#N)cc2)C(=O)C(Cc2ccc(C#N)cc2)C1]

### Synthesis of Intermediate (F1-2) (Scheme 11)

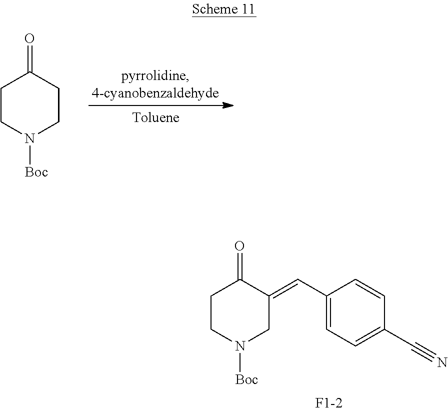 [SMILES: CC(C)(C)OC(=O)N1CCC(=O)CC1 ; CC(C)(C)OC(=O)N1CCC(=O)/C(=C/c2ccc(C#N)cc2)C1]

To a solution of 4-cyanobenzaldehyde (1.88 g, 0.014 mol) in toluene (30 mL) was added pyrrolidine (1.66 mL, 0.02 mol) and the reaction mixture was refluxed for 2 h. After cooling to room temperature, Boc-4-piperidone (2.86 g, 0.014 mol) was added and the mixture was refluxed for 6 h. The mixture was diluted with ethyl acetate, washed with saturated aqueous sodium chloride. The organic phase was dried on anhydrous sodium sulfate and evaporated under reduced pressure. The crude product was purified on silica gel column chromatography (40% EtOAc—hexanes). Yellow solid was isolated (1.5 gr, 35% yield).

¹H-NMR (CDCl₃): 1.43 (s, 9H), 2.37 (t, 4H), 3.65 (t, 4H).

### Synthesis of Intermediate (F1-3) (Scheme 12)

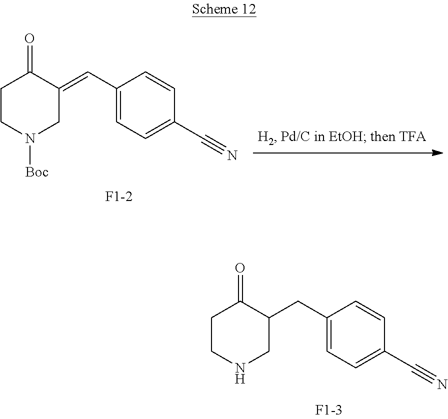 [SMILES: CC(C)(C)OC(=O)N1CCC(=O)/C(=C/c2ccc(C#N)cc2)C1 ; N#Cc1ccc(CC2CNCCC2=O)cc1]

To intermediate F1-2 (1 gr, 3.2 mmol) (Scheme 12) in ethanol (20 mL) was added 10% Pd/C (100 mg, 0.1 w/w %). The reaction was stirred at room temperature under hydrogen atmosphere for 12 h. The reaction mixture filtered through a pad of silica and washed with ethyl acetate. The organic phase was concentrated under reduced pressure. To the crude product was added DCM (15 mL) and TFA (2.5 mL) and the reaction stirred at room temperature for 6 h. TLC showed consumption of starting material. The reaction mixture concentrated to give crude of intermediate F1-3 which was used as such in the next step.

### Synthesis of Intermediate (F1-4) (Scheme 13)

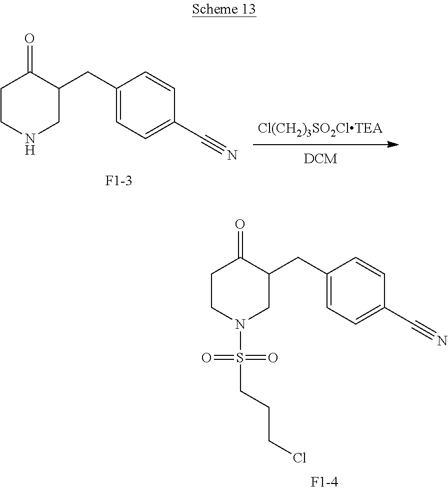 [SMILES: N#Cc1ccc(CC2CNCCC2=O)cc1 ; N#Cc1ccc(CC2CN(S(=O)(=O)CCCCl)CCC2=O)cc1]

Intermediate F1-3 (0.7 g, 2.8 mmol) (Scheme 13) was mixed in DCM (20 mL) and cooled to 0° C. TEA (1.56 mL, 0.01) was added followed by a slow addition of 3-chloropropanesulfonyl chloride (3.3 mmol, 0.4 mL). The reaction was stirred at r.t. for 12 h. Saturated NaHCO₃ (30 mL) was added and the mixture was extracted with DCM (30 mL×3). The combined organic phases were dried on anhydrous sodium sulfate and evaporated under reduced pressure. The crude was purified on silica gel column (0-60% EtOAc—hexanes) to give intermediate F1-4 as a white solid (0.52 g, 53% yield).

### Synthesis of Intermediate (F1-5) (Scheme 14)

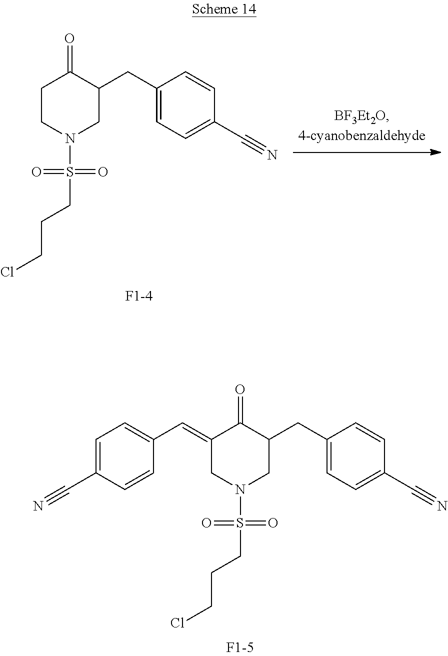 [SMILES: N#Cc1ccc(CC2CN(S(=O)(=O)CCCCl)CCC2=O)cc1 ; N#Cc1ccc(/C=C2\CN(S(=O)(=O)CCCCl)CC(Cc3ccc(C#N)cc3)C2=O)cc1]

Intermediate F1-4 (360 mg, 1 mmol) (Scheme 14) was placed in a 25 ml round bottom flask and cooled to 0° C. Boron trifluoride (5 mL) was added dropwise, and then aldehyde (133 mg, 1 mmol) was added to the reaction mixture in one portion. The reaction was stirred overnight at room temperature under nitrogen atmosphere. The reaction was carefully quenched with a saturated solution of NaHCO₃ and extracted with DCM (30 mL×3). Organic layer was dried on anhydrous sodium sulfate, filtrated and evaporated under reduced pressure. The crude product was purified on silica gel column (20% EtOAc—hexanes) to provide intermediate F1-5 as a yellow colored solid (280 mg, 60% yield).

### Synthesis of Compound F1 (Scheme 15)

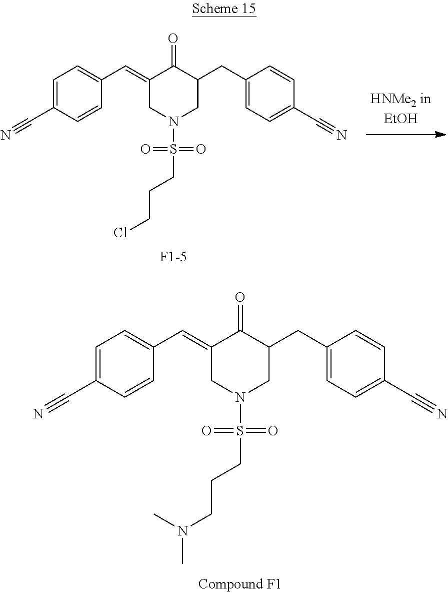 [SMILES: N#Cc1ccc(/C=C2\CN(S(=O)(=O)CCCCl)CC(Cc3ccc(C#N)cc3)C2=O)cc1 ; CN(C)CCCS(=O)(=O)N1C/C(=C\c2ccc(C#N)cc2)C(=O)C(Cc2ccc(C#N)cc2)C1]

Intermediate F1-5 (0.28 gr, 0.6 mmol) (Scheme 15) was dissolved in 5.6M dimethyl amine solution in ethanol (30 mL). Catalytic amount of sodium iodide was added and the reaction stirred at room temperature for 3 days. The solvent was evaporated and the crude mixture was purified on silica gel chromatography (MeOH-DCM gradient up to 20%) to furnish Compound F1 as a yellow colored solid (100 mg, 35% yield).

[M+H]: 477.19 found: 477.2. ¹H NMR (400 MHz, CDCl₃) δ 7.73 (d, J=8.3 Hz, 2H), 7.62 (d, J=8.1 Hz, 2H), 7.63 (s, 1H), 7.44 (d, J=8.3 Hz, 2H), 7.39 (d, J=8.2 Hz, 2H), 4.49 (q, J=15.6 Hz, 2H), 3.64 (dd, J=12.8, 4.4 Hz, 1H), 3.31 (ddd, J=15.3, 12.7, 4.8 Hz, 2H), 3.07-2.91 (m, 4H), 2.37 (t, J=6.6 Hz, 2H), 2.19 (s, 6H), 1.94 (m, 2H).

HPLC: The concentration/purity of major isomer at 9.46 minute RT in HPLC at 225 nM, 254 nM, 270 nM, 285 nM and 325 nM wavelengths showed from 92% to 97.99%. The intensity of the minor isomers were from 0.5% to 5.8%.

### Example 6

### Synthesis of Compound CA

### Synthesis of Intermediate CA-1 (Scheme 16)

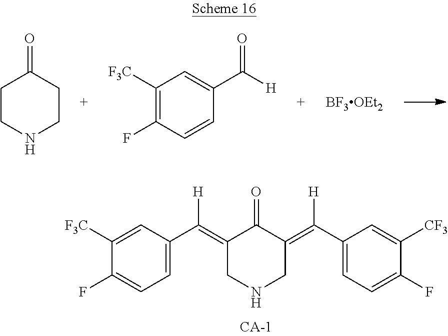 [SMILES: O=C1CCNCC1 ; O=Cc1ccc(F)c(C(F)(F)F)c1 ; O=C1/C(=C/c2ccc(F)c(C(F)(F)F)c2)CNC/C1=C\c1ccc(F)c(C(F)(F)F)c1 ; "BF3·OEt2"]

4-Piperidinone monohydrate hydrochloride (2 gr, 0.013 mol) was placed in a 100 ml round bottom flask and cooled to 0° C. Boron trifluoride etherate (10 mL) was added dropwise followed by aldehyde (5 gr, 0.026 mol) in one portion. The reaction was stirred overnight at room temperature under nitrogen atmosphere. The reaction was carefully quenched with a saturated solution of NaHCO₃. A Yellow solid precipitated out was filtered under reduced pressure, washed with water and EtOH to give intermediate CA-1 (Scheme 16) (3.7 gr, 8.3 mmol, 63%).

### Synthesis of Intermediate CA-2 (Scheme 17)

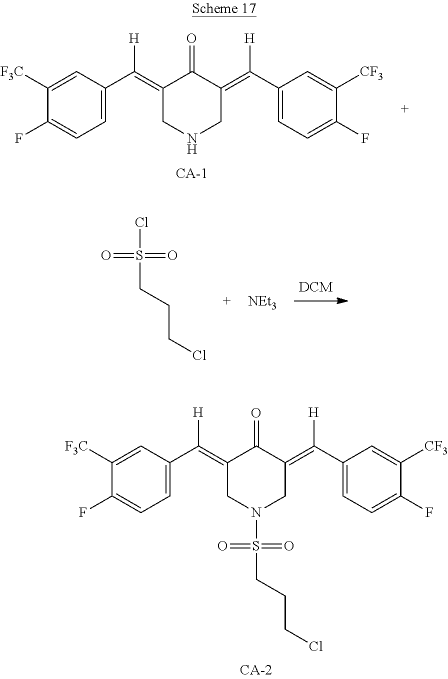 [SMILES: O=C1/C(=C/c2ccc(F)c(C(F)(F)F)c2)CNC/C1=C\c1ccc(F)c(C(F)(F)F)c1 ; O=S(=O)(Cl)CCCCl ; O=C1/C(=C/c2ccc(F)c(C(F)(F)F)c2)CN(S(=O)(=O)CCCCl)C/C1=C\c1ccc(F)c(C(F)(F)F)c1 ; "NEt3"]

Intermediate CA-1 (2 gr, 4.6 mmol) (Scheme 17) and TEA (18 mmol, 2.5 ml) were stirred in DCM (100 ml) under nitrogen atmosphere. The reaction was cooled down to 0° C., sulfonyl chloride (1.22 ml, 6.9 mmol) was added dropwise and the reaction stirred for 3 h. Saturated NaHCO₃ (30 ml) was added to the reaction mixture and extracted with DCM (30 ml×3). The combined organic phases were dried with anhydrous sodium sulfate and evaporated under reduced pressure. The crude was purified by silica gel chromatography (20% EtOAc in hexanes) to give intermediate CA-2 (2.85 gr, 67% yield) (Scheme 17).

### Synthesis of Compound CA (Scheme 18)

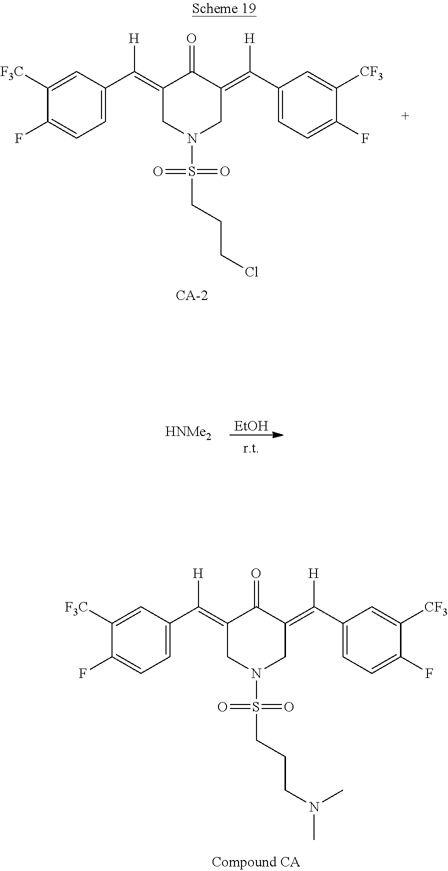 [SMILES: "EtOH" ; O=C1/C(=C/c2ccc(F)c(C(F)(F)F)c2)CN(S(=O)(=O)CCCCl)C/C1=C\c1ccc(F)c(C(F)(F)F)c1 ; CN(C)CCCS(=O)(=O)N1C/C(=C\c2ccc(F)c(C(F)(F)F)c2)C(=O)/C(=C/c2ccc(F)c(C(F)(F)F)c2)C1 ; "HNMe2"]

Intermediate CA-2 (1 gr, 0.9 mmol) (Scheme 18) was dissolved in 5.6M dimethyl amine solution in ethanol (30 ml). Catalytic amount of sodium iodide was added and the reaction was stirred at room temperature for 4 days. The solvent was evaporated and the crude was purified with silica gel chromatography (A=1% TEA in MeOH; B=DCM mixture, gradient of A in B up to 20%) to produce Compound CA (800 mg, 1.34 mmol, 78% yield) (Scheme 18).

¹H NMR (400 MHz, DMSO-d₆) δ 8.00 (d, J=6.6 Hz, 2H), 7.95-7.86 (m, 2H), 7.81 (s, 2H), 7.66 (t, J=9.6 Hz, 2H), 4.64 (s, 4H), 3.22-3.03 (m, 2H), 2.24 (t, J=6.9 Hz, 2H), 2.07 (s, 6H), 1.79-1.63 (m, 2H).

### Example 7

### Synthesis of Compound BA

### Synthesis of Intermediate (BA-2) (Scheme 20)

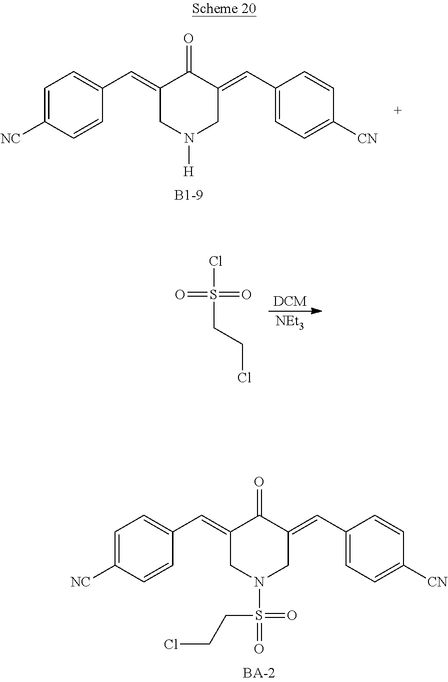

Intermediate B1-9 (0.78 gr, 2.4 mmol) (Scheme 20) and TEA (9.6 mmol, 1.4 ml) were stirred in 25 mL DCM under nitrogen atmosphere. The reaction was cooled down to 0° C., then, sulfonyl chloride (0.38 ml, 3.6 mmol) was added dropwise and the reaction stirred for 3 h at room temperature. Saturated NaHCO₃ (30 ml) was added to the reaction mixture and extracted with 30 ml DCM three times. The combined organic phases were dried with anhydrous sodium sulfate and evaporated under reduced pressure. The crude was purified with silica gel chromatography (20% EtOAc—hexanes). Yellow solid (0.8 gr, 73% yield).

### Synthesis of Compound BA (Scheme 21)

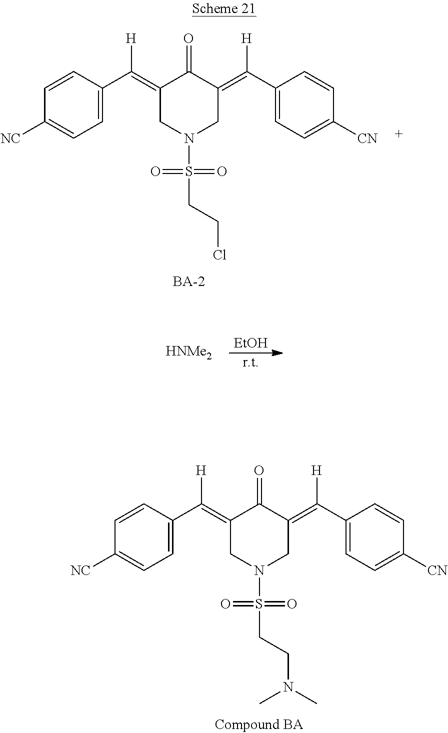 [SMILES: "EtOH" ; N#Cc1ccc(/C=C2\CN(S(=O)(=O)CCCl)C/C(=C\c3ccc(C#N)cc3)C2=O)cc1 ; "r.t." ; CN(C)CCS(=O)(=O)N1C/C(=C\c2ccc(C#N)cc2)C(=O)/C(=C/c2ccc(C#N)cc2)C1 ; "HNMe2"]

Intermediate BA-2 (0.8 gr, 0.9 mmol) (Scheme 21) was dissolved in dimethyl amine solution in ethanol (30 ml 5.6M). Catalytic amount of sodium iodide was added and the reaction stirred at room temperature for 4 days. The solvent evaporated and the crude product was purified with silica gel chromatography (MeOH/DCM mixture, gradient up to 20%). Yellow solid (470 mg, 59% yield). [M+H]: 460.16 found: 461.0, HPLC purity: 95%.

¹H NMR (400 MHz, DMSO) δ 7.99 (d, J=8.3 Hz, 4H), 7.78 (s, 2H), 7.76 (d, J=8.3 Hz, 4H), 4.66 (s, 4H), 3.34 (t, J=6.9 Hz, 2H), 2.24 (t, J=6.9 Hz, 2H), 2.14 (s, 6H).

### Example 8

### Synthesis of Compound B3 and Compound B2

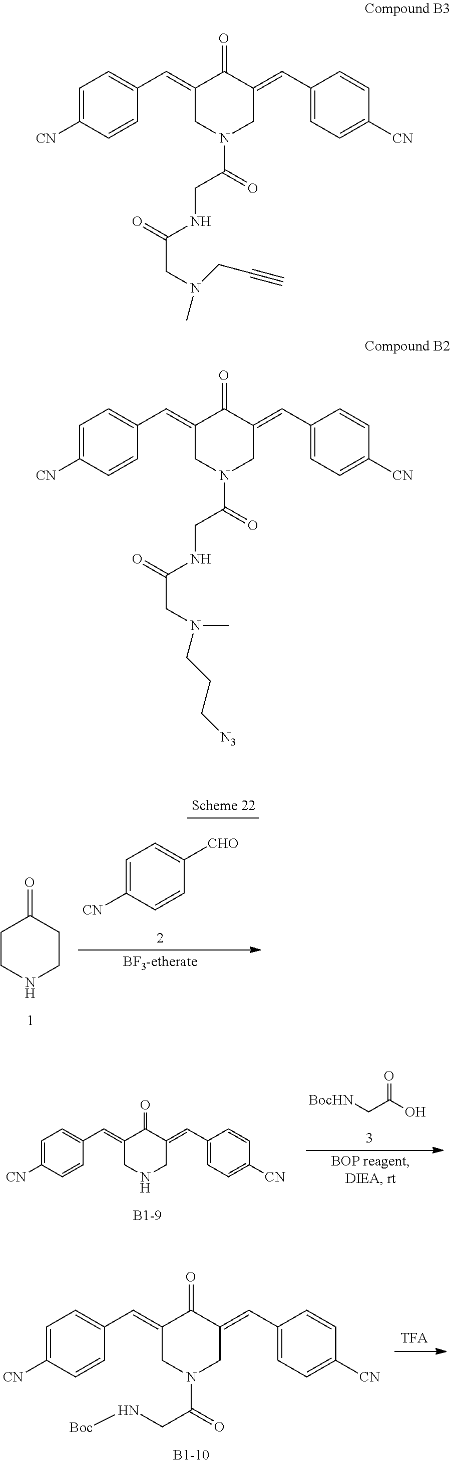

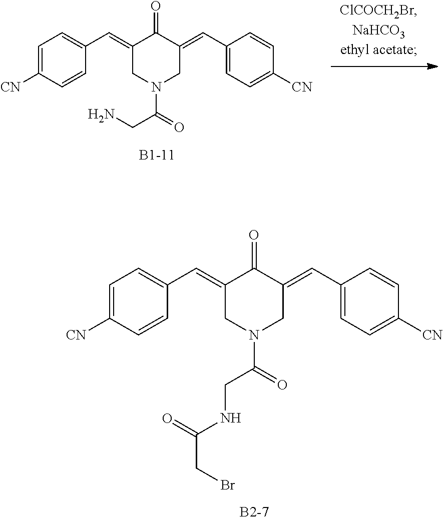

### Synthesis of Intermediate B2-7 (Scheme 22)

Intermediate B1-10 (390 mg) stirred in 1.5 mL of TFA at ambient temperature for 2 h. The reaction mixture was concentrated under reduced pressure to obtain crude compound B1-11. It was suspended in ethyl acetate (5 mL). Saturated sodium bicarbonate solution (5 mL) was added, followed by bromoacetyl chloride (5 eq). The reaction mixture was stirred vigorously for 2 h and the resulting solid collected by filtration, washed with water and ethyl acetate.

### Synthesis of Compound B3 and Compound B2 (Scheme 23)

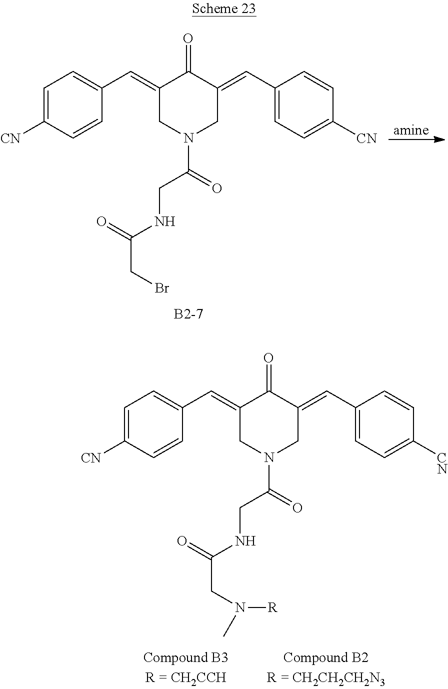

### Synthesis of Compound B3 (Scheme 23)

The starting material B2-7 (621 mg, 1.23 mmol) (Scheme 23), was suspended in DCM (20 mL) and toluene (10 mL). The N-methylpropargylamine (104 mg, 0.127 mL, 1.51 mmol) was added as a solution in 6 mL toluene. The solution was stirred overnight. To the yellow reaction mixture was added 1 mL of saturated NaHCO₃ solution and celite. After evaporation of the mixture to dryness, it was loaded on a combi-flash and colomed, starting from 100% DCM up to 50% EtOAc. The product arrived at 40% EtOAC. Compound B3 was obtained in 125 mg (20% yield).

¹H NMR (DMSO, 400 MHz): δ 2.21 (s, 3H), 2.26 (s, 1H), 2.92 (s, 2H), 3.33 (s, 2H), 3.87 (d, 2H, J=4 Hz), 4.82 (br s, 4H), 7.72-7.78 (m, 7H), 7.97 (d, 4H, J=8 Hz); HPLC purity: 95% (270 nm); MS (ESI+) m/z 492.1 [M+H]⁺.

### Synthesis of Compound B2 (Scheme 23)

The starting material B2-7 (400 mg, 0.8 mmol) (Scheme 23), was suspended in DCM (50 mL). The 3-azido-N-methylpropan-1-amine (181 mg, 1.59 mmol) was added as a solution in DCM (3 mL). The reaction mixture was stirred overnight. The solvent was evaporated and the crude was purified by column chromatography (0 to 20% MeOH-DCM). Compound B2 was obtained in 90 mg (21% yield). HPLC purity: 95%; MS (ESI+) m/z 537.1 [M+H]+.

¹H NMR (400 MHz, DMSO): δ 7.97 (d, J=8.3 Hz, 4H), 7.81-7.65 (m, 7H), 4.83 (s, 4H), 3.90 (d, J=5.5 Hz, 2H), 3.37-3.32 (m, 2H), 2.86 (s, 2H), 2.38 (t, J=6.9 Hz, 2H), 2.16 (s, 3H), 1.72-1.52 (m, 2H).

### Example 9

### Synthesis of Compounds B4-B7

### Synthesis of Compound B5

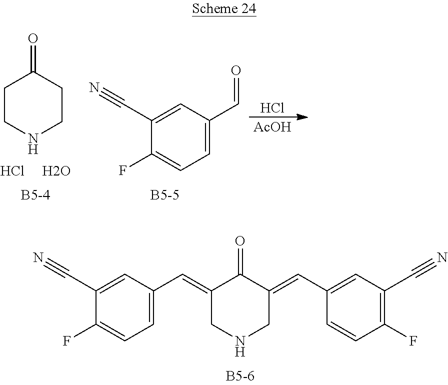

Reaction of 4-piperidone (B5-4) with two equivalents of 2-fluoro-5-formylbenzonitrile (B5-5) in glacial acetic acid afforded Intermediate B5-6 (Scheme 24).

### 5-{[(3E,5E)-5-[(3-cyano-4-fluorophenyl)methylidene]-4-oxopiperidin-3-ylidene]methyl}-2-fluorobenzonitrile (B5-6) (Scheme 24)

Hydrochloric acid was bubbled into a solution of 4-piperidone monohydrate hydrochloride (B5-4) (1.5 g, 1 eq) in acetic acid (15 ml) at r.t. for 15 min. Then 2-fluoro-5-formylbenzonitrile (B5-5) (2.9 g, 2 eq) was added and stirred for 12 h at r.t. LC-MS analysis indicated that starting material was consumed. The mixture was filtered and the solid was washed with ethanol, diethyl ether and dried in vacuum to obtain B5-6 as a yellow solid (1.17 g, 33% yield) (Scheme 24). HPLC purity: 99%; MS (ESI+) m/z 460.2 [M+H]+.

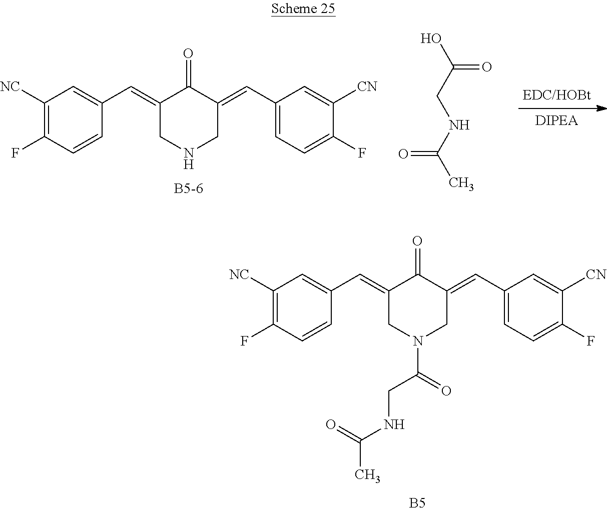

Intermediate B5-6 was coupled with N-acetyl glycine (Scheme 25) in the presence of EDC (1-Ethyl-3-(3-dimethylaminopropyl) carbodiimide) and HOBt (1-Hydroxybenzotriazole hydrate) to obtained Compound B5 (Scheme 25).

### N-{2-[(3E,5E)-3,5-bis[(3-cyano-4-fluorophenyl)methylidene]-4-oxopiperidin-1-yl]-2-oxoethyl}acetamide (Compound B5) (Scheme 25)

N-(3-Dimethylaminopropyl)-N′-ethylcarbodiimide hydrochloride (EDC) (0.95 g, 1.2 eq) and N, N-diisopropylethylamine (0.217 ml, 2.5 eq) were added to acid N-acetyl glycine (50 mg, 1 eq) and 1-Hydroxybenzotriazole hydrate (HOBt) (202 mg, 1.2 eq) in DMF (10 ml) and stirred for 20 min, then B5-6 (0.15 g, 1 eq) was added and stirred overnight under N₂. The reaction mixture was heated to 50° C. for 10 h. The compound was purified by preparative HPLC (XBridge C18 column, gradient of AcCN in 50 mM NH4HCO3). The purest fractions were concentrated in vacuo and the residue was dried under high vacuum to give light yellow solid (11.7 mg, 12% yield). HPLC purity: 97%; MS (ESI+) m/z 461 [M+H]+.

### Synthesis of Compounds B6 and B7 (Scheme 26)

N-acetyl glycine and N-acetyl L-serine were coupled with Compound B1-9 using HATU to give the respective amide products Compound B6 and Compound B7 (Scheme 26).

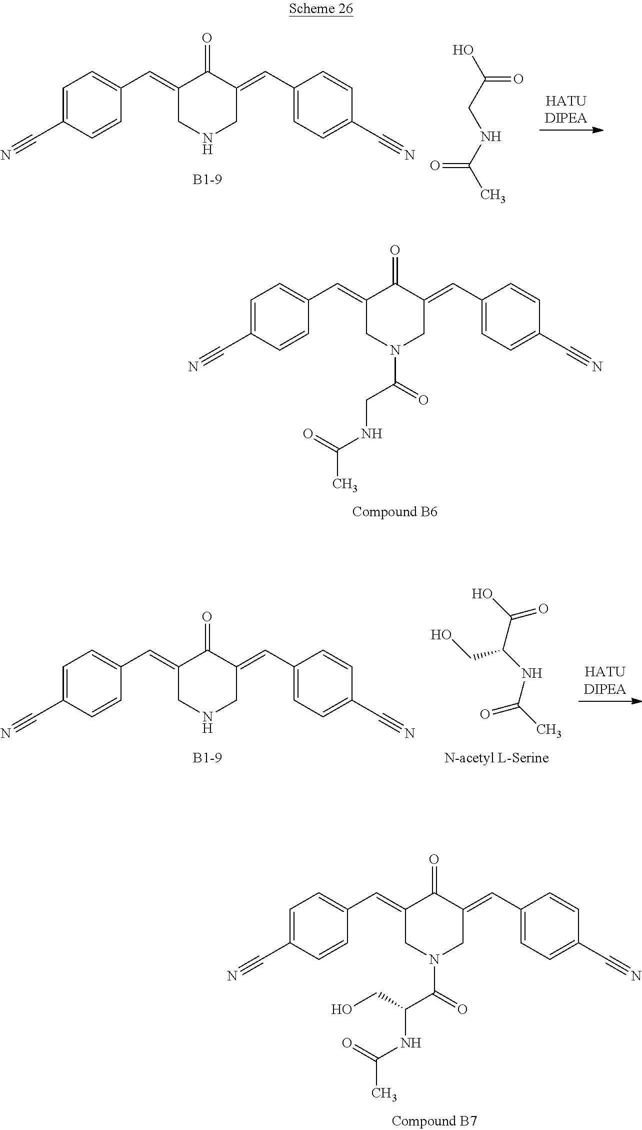

### N-{2-[(3E,5E)-3,5-bis[(4-cyanophenyl)methylidene]-4-oxopiperidin-1-yl]-2-oxoethyl}acetamide (Compound B6) (Scheme 26)

To a solution of glycine N-acetate (66.0 mg, 1.1 eq) in 1.5 ml DMF were added DIPEA (0.39 ml, 4.6 eq) and HATU (261 mg, 1.4 eq). After 2 min at r.t. B1-9 (207 mg, 1 eq) was added in 4 ml DMF. The reaction mixture was stirred at r.t. for 2 h. The product was observed by LC-MS and water were added. The product was extracted with ethyl acetate ×3 and was washed with brine ×2 and water. The combined organic phases were dried with anhydrous Na₂SO₄, filtered and evaporated. The yellow solid was dried under high vacuum overnight. Water were added to the mixture and the mixture was filtered under vacuum. The crude was dissolved in 12 ml ACN and 1 ml 1,4-dioxane and was purified by prepara-tive HPLC (ACE C8 column, gradient of ACN in 0.1% TFA water). The purest fractions were concentrated under vacuum to give the product as a yellow solid (40.2 mg, 19% yield). HPLC purity: 99%; MS (ESI+) m/z 425 [M+H]+.

### N-[(2R)-1-[(3E,5E)-3,5-bis[(4-cyanophenyl)methylidene]-4-oxopiperidin-1-yl]-3-hydroxy-1-oxopropan-2-yl]acetamide (Compound B7) (Scheme 26)

To a solution of N-acetyl-L-serine (75.5 mg, 1.1 eq) in 7 ml DMF, DIPEA (0.34 ml, 4.1 eq) and HATU (247.6 mg, 1.4 eq) were added. After 2 min at r.t. B1-9 (201.3 mg, 1 eq) was added in 3 ml DMF. The reaction mixture was stirred at r.t. for 1 h. According to LC-MS, a new peak as obtained. Next, the product was washed with brine and extracted with ethyl acetate (×3). The combined organic phases were evaporated. Water were added to the mixture and the mixture was filtered under vacuum. The crude was dissolved in 15 ml ACN and 1.5 ml 1,4-dioxane and was purified by preparative HPLC (ACE C8 column, gra-dient of ACN in 0.1% TFA water). The purest fractions were concentrated under vacuum to give the product as a yellow solid (37 mg, 17% yield). HPLC purity: 99%; MS (ESI+) m/z 455 [M+H]+.

### Example 10

### Synthesis of Compounds B8

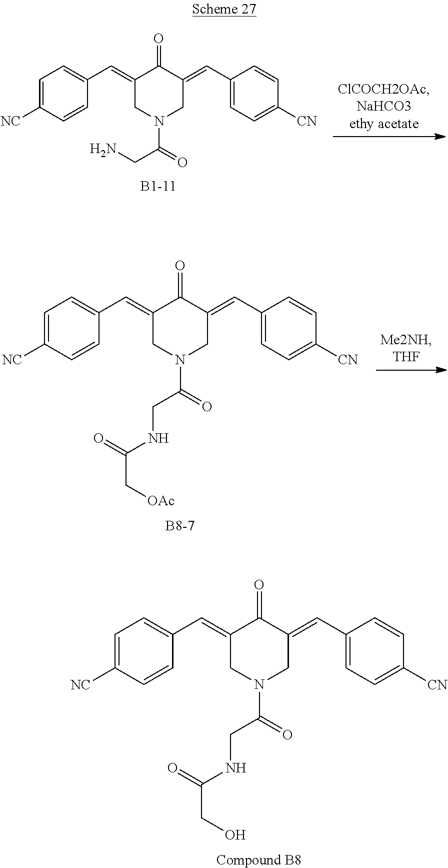

### Synthesis of Compound B8-7

Compound B1-11 (1 g) (Scheme 27) was stirred in 3 mL of TFA at ambient temperature for 2 h. The reaction was concentrated and the residue dissolved in 10 mL ethyl acetate, followed by addition of 10 mL saturated sodium bicarbonate solution. To the reaction mixture was added 5 eq of acetoxyacetyl chloride. After stirring for 2 h the resulting solid was collected by filtration, washed with water ethyl acetate. Upon drying, 508 mg of the acetate compound B8-7 (Scheme 27) was obtained.

### Synthesis of Compound B8 (Scheme 27)

Compound B8-7 (Scheme 27) was dissolved in 5 mL of DCM and 1 eq of dimethylamine (2.0 M solution in THF) was added. Upon stirring 2 h at ambient temperature the reaction mixture was concentrated and the residue purified by column chromatography to give 124 mg of final product (Scheme 27).

¹H NMR (DMSO-d6, 400 MHz): δ 3.73 (d, 2H, J=4 Hz), 3.89 (d, 2H, J=4 Hz), 4.80 (d, 4H, J=9 Hz), 5.52 (t, 1H, J=4 Hz), 7.66 (m, 8H), 7.95 (d, 4H J=4 Hz); HPLC purity: 97%; MS (ESI+) m/z 441.16 [M+H]+.

### Example 11

### Biological Activity of Compounds of the Invention

### Experimental Methods

Cell Viability. The assay was performed only when cell viability was ≥90%. Cells were seeded in 96-well white clear bottom plates at concentration of 100,000 cells/mL and treated with serially diluted compounds or vehicle (DMSO) control in triplicates for 48 hours (h). Cell viability was determined using the ATPlite 1-step assay system (PerkinElmer). The method was based on the production of light caused by the reaction of ATP, which was a marker for cell viability. Luciferin and its substrate were added to the plates which were read in a plate reader for luminescence. The emitted light is proportional to the ATP concentration. Viability was calculated as the percent of viable cells from control vehicle-treated cells. EC₅₀ was calculated using Prism software.

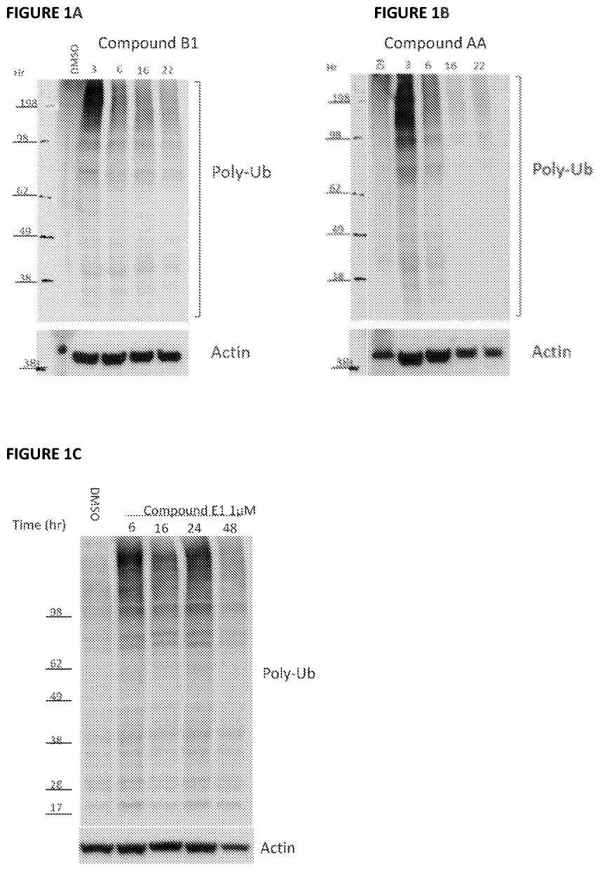

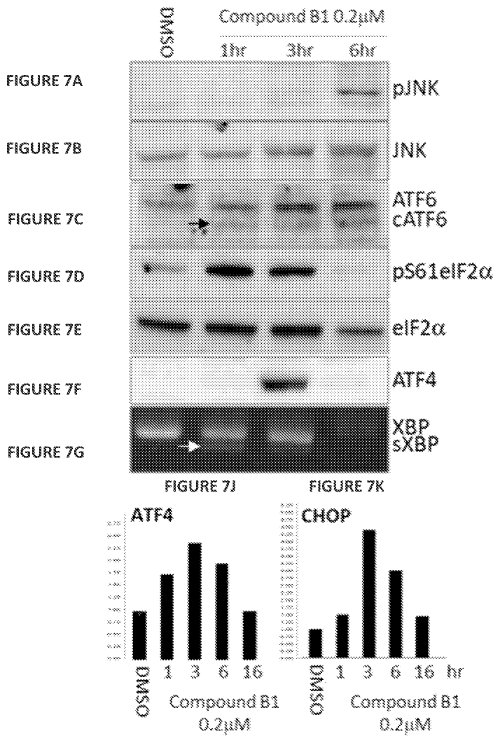

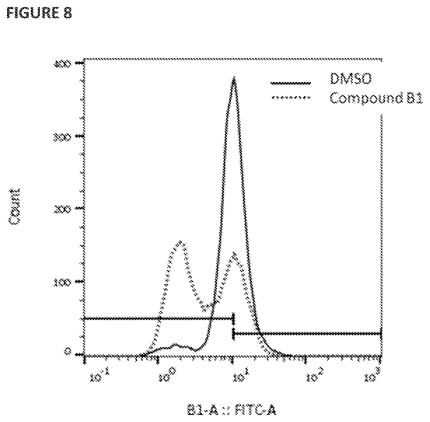

Protein analysis by Western blot. Cells (100,000 cells/ml) were treated with compounds or vehicle control as indicated in FIGS. 1 and 7. At the end of treatment period, the cells were lysed with M-PER mammalian protein extraction reagent (ThermoFisher Scientific) supplemented with protease inhibitors. Equal protein amounts were resolved on pre-cast SDS-PAGE (ThermoFisher Scientific) and transferred to a PVDF membrane. The membrane was immunoblotted with antibodies as indicated. The following antibodies were used: Ub MAb (SC-8017, Santa-Cruz), ATF4, ATF6, phospho JNK, JNK (Cell signaling); eIF2alpha, phospho eIF2alpha (Novus).

Proteasome activity. The assay was performed only when cell viability is ≥90%. Cells were seeded in 96-well white clear bottom plates and treated with diluted compounds or vehicle control in triplicates for 3 hours at various concentrations. Catalytic activity was measured using three luminogenic proteasome substrates: Suc-LLVY-aminoluciferin (Succinyl-leucine-leucine-valine-tyrosine-aminoluciferin), Z-LRR-aminoluciferin (Z-leucine-arginine-arginine-aminoluciferin) and Z-nLPnLD-aminoluciferin (Z-norleucine-proline-norleucine-aspartate-aminoluciferin) for the chymotrypsin-like, trypsin-like and caspase-like activities, respectively (Proteasome-GLO, Promega). The emitted light was proportional to the proteasomal activity. Catalytic activity was calculated as the percent activity from control vehicle-treated cells.

Kinetic solubility quantification. Compound B1/Compound E1 DMSO stock was diluted with PBS by serial 2-fold dilutions. Then, the samples were centrifuged for 5 minutes at 17,000 g to precipitate any insoluble compound. Each sample was tested at a single wave length (Compound B1 at 330 nM; Compound E1 at 315 nM) in duplicate before and after centrifugation to assess solubility of the compound. Soluble concentrations were determined when optical density (OD) was equivalent between centrifuged and non-centrifuged fractions.

Animal xenograft models. MM1.S, HCT-116, SW620 cell lines were purchased from ATCC and used for xenograft model. The cells were cultured in RPMI medium (Sigma-Aldrich) supplemented with 10% fetal bovine serum (FBS) and divided for up to 5 passages. Shortly prior to injection, cell suspension was mixed with Matrigel at 1:1 (V/V) and injected subcutaneously to the rear flank area of male nude athymic 6 weeks old mice to obtain administration of 5×10⁶ cells/animal. On day 20-23, when tumor volume reached 100-150 mm³, mice were randomized for equivalent distribution of tumor volumes to treatment groups (n=5/group) and treated via intravenous injection.

Cell viability panel studies. Cells were diluted in the corresponding ATCC recommended medium and dispensed in a 384-well plate, depending on the cell line used, at a density of 200-6400 cells per well. For each used cell line, the optimal cell density was used. Compound was serially diluted and 8 concentration (0.04-32 μM) were added to the cells for 72 h exposure. At t=end, of ATPlite 1Step™ (PerkinElmer) were used to calculate cell viability. Cell lines marked with asterisk were seeded 24 h prior to treatment in 96-well plates at the density range of 5,000-40,000 cells/well, according to duplication rate, in RPMI 1640 medium containing 10% fetal bovine serum and 2 mM L-glutamine. Compounds were diluted from 10 mM DMSO stock and treatment was applied at the range of 0.04-1 μM. Following incubation at 37° C. for 48 hours with increasing concentrations of the compounds, viability was determined with CyQUANT® Direct Assay Kit (Invitrogen). Output intensities were normalized to values after treatment with DMSO alone, and EC₅₀ values were calculated using the absorbance measurements [time zero, (Tz), growth control, (C), plus the test growth at the four drug concentration levels (Ti)] as follows: [(Ti−Tz)/(C−Tz)]×100 for concentrations for which Ti>/=Tz; [(Ti−Tz)/Tz]×100 for concentrations for which Ti−<Tz.

RT-PCR: MM cells were treated with compounds as indicated. Total RNA was extracted using RNAeasy kit (QIAGEN) and cDNA was synthesized using reverse transcriptase (Quantaces biosciences). mRNA levels of ATF4 and CHOP were determined by quantitative PCR using a StepOnePlus™ Real Time PCR system (Life Technologies) with gene specific assays (Thermo Scientific). XBP splicing were addressed by differential migration of XBP-1 gene transcript full size versus spliced form on 2% Agarose gel.

Autophagy Quantification: Quantification of autophagosomes were done with CytoID (ENZO). CYTO-ID® Autophagy Detection Kit measures autophagic vacuoles and monitors autophagic flux in live cells using a dye that selectively labels autophagic vacuoles. The probe is a cationic amphiphilic tracer dye that rapidly partitions into cells in a similar manner as drugs that induce phospholipidosis. MM1.S cells were treated with Compound B1 or vehicle for 5 hours. Following treatment period the cells were harvested and stained with CytoID dye according to manufacturer instructions. Autophagosomes were analyzed and quantified using flow cytometer (Miltenyi). Data of cell counts were plotted as FITC (FL1) fluorescence intensity.

### Results

### Compound B1 is Cytotoxic to Multiple Types of Cancer Cells.

As shown in Table 1, Compound B1 was shown to exhibit cytotoxicity upon exposure of a variety of cancer cells. Table 1 shows the associated potency upon treating cancer cell lines with Compound B1 generated from the NCI60 screen (as described in, e.g., Nature Reviews Cancer 6, 813-823 (October 2006), which is hereby incorporated by reference in its entirety).

TABLE 1
Associated potency upon treating cancer
cell lines with Compound B1

| | | Compound B1 EC₅₀ |
|---|---|---|
| | | +++: EC₅₀ < 0.3 μM; |
| | | ++: 0.3 μM ≤ EC₅₀ < 1 μM; |
| Cancer type | Cell line | +: EC₅₀ ≥1 μM |
| Breast cancer | MDA-MB-231 | +++ |
| Breast cancer | MDA-MB-468 | +++ |
| Breast cancer | BT-549 | +++ |
| Breast cancer | HS-578T | ++ |
| Breast cancer | MCF-7 | ++ |
| Colon cancer | KM-12 | +++ |
| Colon cancer | SW-620 | +++ |
| Colon cancer | HCT-116 | +++ |
| Colon cancer | HCC-2998 | +++ |
| Colon cancer | HCT-15 | +++ |
| Colon cancer | HT-29 | ++ |
| Colon cancer | COLO-205 | ++ |
| Leukemia | CCRF-CEM | +++ |
| Leukemia | MOLT-4 | +++ |
| Leukemia | HL-60 | +++ |
| Leukemia | K-562 | +++ |
| Lung cancer | HOP-62 | +++ |
| Lung cancer | NCI-H23 | +++ |
| Lung cancer | NCI-H522 | +++ |
| Lung cancer | NCI-H460 | +++ |
| Lung cancer | A-549 | ++ |
| Lung cancer | HOP-92 | ++ |
| Lung cancer | EKVX | ++ |
| Lung cancer | NCI-H322M | ++ |
| Ovarian cancer | OVCAR-3 | +++ |
| Ovarian cancer | SK-OV-3 | +++ |
| Ovarian cancer | OVCAR-8 | +++ |
| Ovarian cancer | IGR-OV1 | +++ |
| Ovarian cancer | OVCAR-5 | +++ |
| Ovarian cancer | OVCAR-4 | ++ |
| Renal cancer | TK-10 | ++ |
| Renal cancer | UO-31 | ++ |
| Renal cancer | 786-0 | ++ |
| Renal cancer | ACHN | +++ |
| Renal cancer | SN12C | +++ |
| Prostate cancer | PC-3 | +++ |
| Prostate cancer | DU-145 | +++ |
| Melanoma | LOX IMVI | +++ |
| Melanoma | MALME-3M | +++ |
| Melanoma | M14 | +++ |
| Melanoma | MDA-MB-435 | +++ |
| Melanoma | SK-MEL-2 | +++ |
| Melanoma | SK-MEL-28 | ++ |
| Melanoma | SK-MEL-5 | ++ |
| Melanoma | UACC-257 | ++ |
| Melanoma | UACC-62 | ++ |
| CNS | SF-268 | +++ |
| CNS | SF-295 | +++ |
| CNS | SF-539 | ++ |
| CNS | SNB-19 | ++ |
| CNS | SNB-75 | ++ |
| CNS | U251 | +++ |

### Compound B1, Compound AA and Compound E1 Induces the Accumulation of Poly-Ubiquitinated Proteins.

MM1.S cells treated with Compound B1, Compound AA or Compound E1 had an observable accumulation of poly-ubiquitinated proteins, a result which is a hallmark of UPS inhibition (FIG. 1A-1C).

Compound B1 and Compound AA does not Inhibit the Enzymatic Functions of the Proteasome.

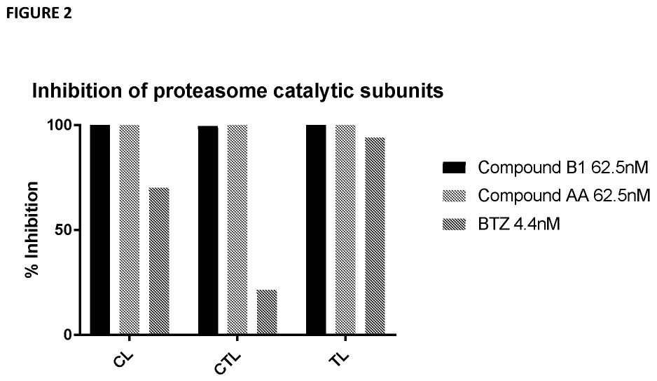

MM1.S cells, were treated with Compound B1, Compound AA or Bortezomib (BTZ) at various concentrations for 3 hr at 37° C. (FIG. 2). Proteasome activity was measured by cleavage of proteasome-specific peptide substrates for TL, CTL and PL activities. Inhibition of proteasome was detected only by BTZ, which specifically inhibits CTL activity

### Kinetic Solubility of Compound B1 and Compound E1.

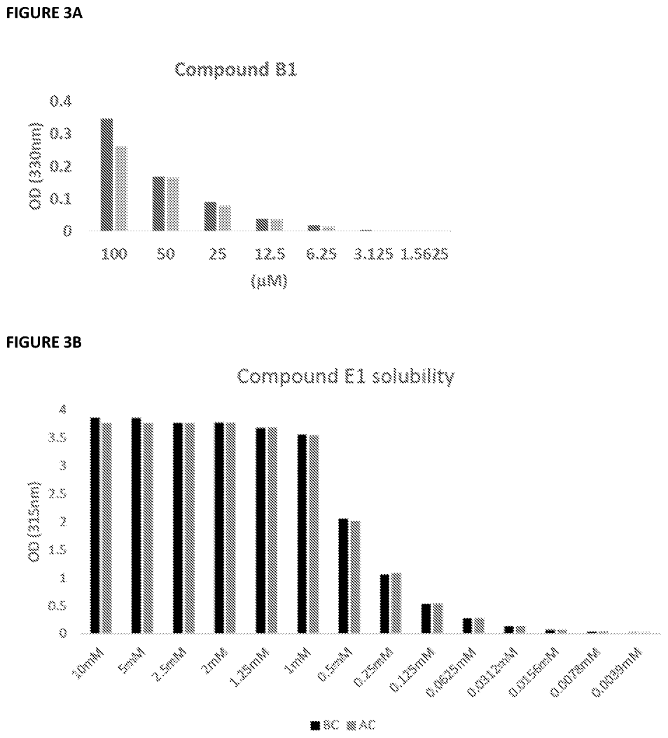

Kinetic solubility of all Compounds was determined by differential UV absorbance before and after centrifugation. Compound B1, and Compound E1 possessed a clear UV signature (measured at 310-360 nm), which may be used for compound detection. Solubility concentration was determined when OD was equivalent between centrifuged and non-centrifuged fractions (FIG. 3A-3B). Compound 131 and Compound E1 solubility is 50 μM and 2.5 mM respectively

### In-Vivo Efficacy of Compound B1 and Compound a in a Multiple Myeloma (MM) Subcutaneous Flank Xenografts in Athymic Nude Mice.

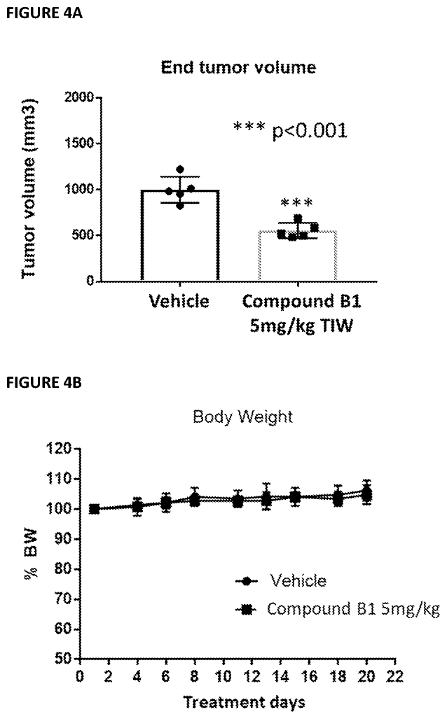

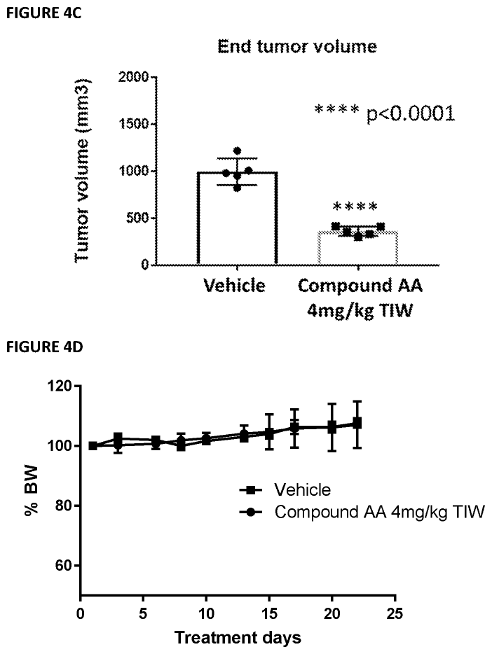

Treatment of tumor-bearing mice with 5 mg/kg Compound B1 and 4 mg/kg Compound AA significantly inhibited MM tumor growth compared to vehicle control (FIG. 4A, FIG. 4C). Blood chemistry profiles of Compound B1 and Compound AA treated mice showed no clinical abnormalities suggestive of liver or kidney toxicity. In addition, animal body weight was not considerably affected by the treatment (FIG. 41, FIG. 4D). FIG. 4A, FIG. 4C show tumor growth inhibition observed at end point measurements. FIG. 4B, FIG. 4D show the body weight % changes in treated animals. No significant weight loss was observed in mice treated with Compound B1 at 5 mg/k-g and Compound AA at 4 mg/kg.

In-Vitro Safety in PBMCs from Healthy Donors.

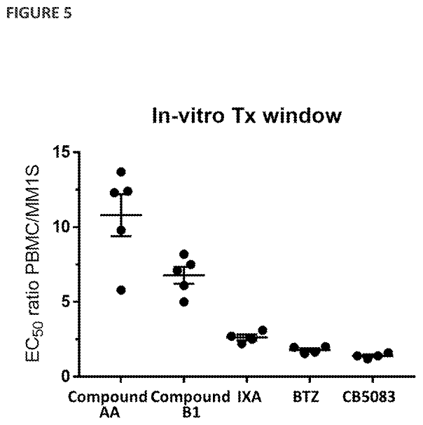

PBMCs from healthy donors were exposed to Compound B1 and Compound AA for 6 hr and analyzed for viability by ATPight following 48 h of incubation. Results are representative of PBMCs from 5 healthy volunteers (FIG. 5). EC₅₀ (PBMC)/EC₅₀ (MM1.S) ratio, generated from 5 healthy donor PBMC samples, is shown for Compound B1, Compound AA and other UPS inhibitors [ixazomib, Bortezomib (BTZ) and CB5083]. MM1.S cells were more sensitive to Compound I and Compound AA than PBMCs from healthy donors. FIG. 5 shows that under current assay settings, Compound B1 and Compound AA have larger Tx window than competing UPS inhibitors suggesting an improved therapeutic window for Compound B1 and Compound AA compared to clinical proteasome inhibitors.

### Beyond MM—Compound AA Potently Targets Additional Hematologic and Solid Tumors Cell Lines

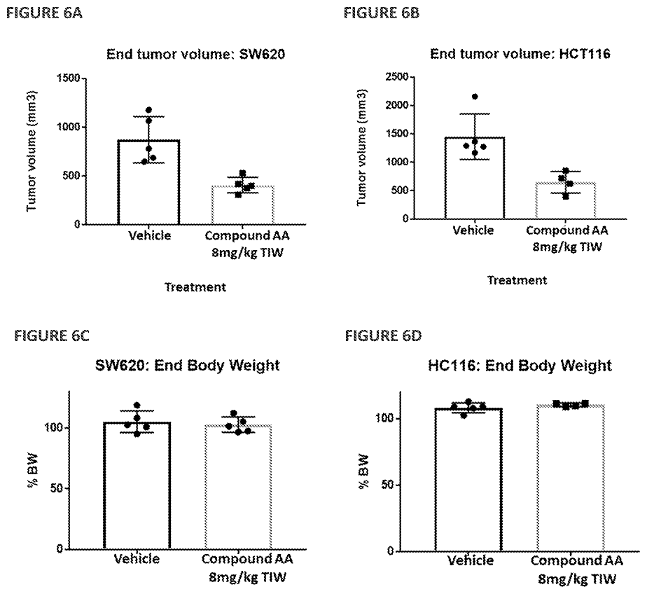

Colon cancer was chosen according to results from viability screening panel with Compound AA. Two cell lines, HCT-116 and SW620, were selected to represent the above indication. Treatment of tumor-bearing mice with 8 mg/kg Compound AA significantly inhibited tumor growth compared to vehicle control (FIG. 6A, FIG. 6B). Animal body weight was not considerably affected by the treatment (FIG. 6C, FIG. 6D).

### Compound AA was Cylotoxic to Multiple Types of Cancer Cells.

### Efficacy of Compounds of the Invention in MM Cells:

TABLE 2
Associated EC₅₀ values upon treatment of MM
cell lines with compounds of the invention

| | | Compound EC₅₀ |
|---|---|---|
| | | +++: EC₅₀ < 0.3 μM; |
| | | ++: 0.3 μM ≤ EC₅₀ < 1 μM; |
| Compound # | Cell line | +: EC₅₀ ≥ 1 μM |
| B9 | U266 | + |
| B10 | U266 | + |
| B11 | U266 | + |
| B12 | U266 | ++ |
| B13 | MM1S | +++ |
| B14 | U266 | ++ |
| B15 | U266 | + |
| B16 | MM1S | +++ |
| B17 | U266 | +++ |
| B18 | U266 | +++ |
| B19 | U266 | +++ |
| B20 | U266 | +++ |
| B21 | U266 | ++ |
| B22 | U266 | +++ |
| B23 | MM1S | +++ |
| B24 | U266 | ++ |
| B25 | U266 | +++ |
| B26 | MM1S | + |
| B27 | U266 | + |
| B28 | U266 | +++ |
| B29 | N/A | + |
| B30 | U266 | +++ |
| B3 | U266 | +++ |
| B4 | MM1.S | +++ |
| B32 | U266 | +++ |
| B5 | U266 | +++ |
| B6 | MM1.S | +++ |
| B7 | U266 | ++ |
| B1 | MM1.S | +++ |
| B8 | MM1.S | +++ |
| CA | MM1.S | +++ |
| BA | MM1.S | +++ |
| B3 | MM1.S | +++ |
| B2 | MM1.S | +++ |
| F1 | MM1.S | +++ |
| D1 | U266 | +++ |
| E1 | MM1.S | +++ |

MM cell lines exhibit differential cytotoxicity upon exposure to the compounds of the invention. Potency of compounds were assessed by viability assay. Table 2 shows the associated EC₅₀ values upon treatment of MM cell lines (U266 and MM1.S)

### Example 12

### UPR Activation by Compounds of the Invention—Mechanistic Investigation

The UPR is initiated by three ER transmembrane proteins: Inositol Requiring 1 (IRE1), PKR-like ER kinase (PERK), and Activating Transcription Factor 6 (ATF6). Upon activation of UPR, a cascade of signaling events is initiated. Those will eventually regulate both survival and death factors that govern whether the cell will live or not depending on the severity of the ER stress condition. To characterize the UPR activation by compounds of the invention, major signaling events were tested. Endogenous expression levels of PERK and IRE1 molecules are low and hard to detect. Thus, alternatively, it is acceptable to measure expression and activation levels of downstream components. Measuring eIF2a phosphorylation levels by immunoblot using anti-phospho-eIF2a-specific antibody indirectly reflects PERK activation. MM1.S incubation with Compound B1 increases eI2Falpha phosphorylation after 1 hr of treatment. Phosphorylated eIF2apha triggers global mRNA translation attenuation. This reduction in ER workload protects cells from ER stress mediated apoptosis (Harding et al., 2000). Meanwhile, some mRNAs require eIF2alpha phosphorylation for translation such as the mRNA encoding ATF4. ATF4 is a b ZIP transcription factor that regulates several UPR target genes, including those involved in ER stress-mediated apoptosis such as C/EBP homologous protein (CHOP; Harding et al., 2000). Compound B1 treatment leads to a transcriptional increase of both ATF4 and CHOP, peaking at 3 hr post treatment. IRE1, a type I ER transmembrane kinase, senses ER stress by its N-terminal luminal domain (Urano et al., 2000). Upon sensing the presence of unfolded or misfolded proteins, IRE1 dimerizes and autophosphorylates to become active. Activated IRE1a splices X-box binding protein 1 (XBP-1) mRNA (Calfon et al., 2002; Shen et al., 2001; Yoshida et al., 2001). Spliced XBP-1 mRNA encodes a basic leucine zipper (b-ZIP) transcription factor that upregulates UPR target genes, including genes that function in ERAD such as ER-degradation-enhancing-a-mannidose-like protein (EDEM; Yoshida et al., 2003), as well as genes that function in folding proteins such as protein disulfide isomerase (PDI; Lee et al., 2003a). High levels of chronic ER stress can lead to the recruitment of TNF-receptor-associated factor 2 (TRAF2) by IRE1 and the activation of apoptosis-signaling-kinase 1 (ASK1). Activated ASK1 activates c-Jun N-terminal protein kinase (JNK), which in turn plays a role in apoptosis by regulating the BCL2 family of proteins (Nishitoh et al., 1998, 2002; Urano et al., 2000b).

Following Compound B1 treatment, there is a significant upregulation in phosphorylated JNK, without changes in total protein levels. Spliced XBP was detected by differentiated migration of XBP gene transcript along with upregulation of TXNDC5 and PIK3R genes (RNAseq, Diag2Tec, data not shown). This gene encodes a member of the disulfide isomerase (PDI) family of ER proteins that catalyze protein folding and thiol-disulfide interchange reactions, regulated by spliced XBP. PIK3R modulates the cellular response to ER stress by promoting nuclear translocation of XBP. A third regulator of ER stress signaling is the type II ER transmembrane transcription factor, ATF6 (Yoshida et al., 1998). ATF6 has been extensively studied in the context of ER stress. Upon ER stress conditions, ATF6 transits to the Golgi where it is cleaved by site 1 (S1) and site 2 (S2) proteases, generating an activated b-ZIP factor (Ye et al., 2000). This processed form of ATF6 translocates to the nucleus to activate UPR genes involved in protein folding, processing, and degradation (Haze et al., 1999; Yoshida et al., 2000). Compound B1 treatment causes short term upregulation of ATF6 full size form followed by a rapid decline.

The UPS and autophagy are two distinct but interacting proteolytic systems. Aggregated proteins failing to undergo proteasomal degradation may be sequestered by autophagosomes and delivered to lysosomes for clearance. Autophagy, which is largely considered cytoprotective in cancer cells, may thus compensate for UPS inhibition.

FIG. 8 shows a quantitative FACS analysis of autophagosomal vesicles in Compound B1 treated cells vs. vehicle control. MM1.S cells, treated with Compound B1 for 5 hours demonstrate significantly lower fluorescent dye then vehicle treated cells, which indicative to reduced autophagy.

### Example 13

### The Effect of Compound AA on Various Types of Cancer Cells

The compounds of the invention are cytotoxic to cancer cells in-vitro. Table 3 shows the effect of Compound AA treatment on a panel of cancer cells representing different tumor types.

TABLE 3
Efficacy screen performed with Compound
AA on a panel of cancer cell lines

| | | Compound EC₅₀ |
|---|---|---|
| | | +++: EC₅₀ < 0.3 μM; |
| | | ++: 0.3 μM ≤ |
| | | EC₅₀ < 1 μM; |
| Cancer type | Cell line | +: EC₅₀ ≥ 1 μM |
| Acute monocytic leukemia | THP-1 | ++ |
| Acute myeloid leukemia | HL-60 | +++ |
| Acute myeloid leukemia | KG-1 | +++ |
| Alveolar rhabdomyosarcoma | SJCRH30 | +++ |
| Amelanotic melanoma | A375 | +++ |
| Anaplastic large cell lymphoma | SR | +++ |
| Anaplastic large cell lymphoma | SU-DHL-1 | +++ |
| Astrocytoma | CCF-STTG1 | +++ |
| Astrocytoma | U-118 MG | +++ |
| B acute lymphoblastic leukemia | RS4-11 | +++ |
| Biphasic synovial sarcoma | SW982 | +++ |
| Bladder carcinoma | 5637 | +++ |
| Bladder carcinoma | J82 | ++ |
| Bladder carcinoma | RT4 | ++ |
| Bladder carcinoma | T24 | +++ |
| Bladder carcinoma | TCCSUP | +++ |
| Breast adenocarcinoma | AU-565 | +++ |
| Breast Cancer | MDA-MB- | +++ |
| | 231* | |
| Breast cancer | MDA-MB- | +++ |
| | 468* | |
| Breast carcinoma | DU4475 | ++ |
| Cecum adenocarcinoma | LS411N | ++ |
| Cecum adenocarcinoma | SNU-C2B | ++ |
| Cervical carcinoma | DoTc2 4510 | +++ |
| Cervical squamous cell carcinoma | C-33 A | +++ |
| Chronic myelogenous leukemia | K-562 | +++ |
| Chronic myelogenous leukemia | KU812 | +++ |
| CNS | SF-268* | +++ |
| CNS | SF-295* | +++ |
| CNS | SF-539* | +++ |
| CNS | SNB-19* | +++ |
| CNS | SNB-75* | ++ |
| CNS | U251* | +++ |
| Colon adenocarcinoma | COLO 205 | +++ |
| Colon adenocarcinoma | DLD-1 | +++ |
| Colon adenocarcinoma | HCT-15 | ++ |
| Colon adenocarcinoma | LoVo | +++ |
| Colon adenocarcinoma | LS 174T | +++ |
| Colon adenocarcinoma | SW48 | ++ |
| Colon adenocarcinoma | SW480 | +++ |
| Colon adenocarcinoma | SW620 | +++ |
| Colon adenocarcinoma | SW626 | +++ |
| Colon adenocarcinoma | SW948 | +++ |
| Colon cancer | HCC-2998* | ++ |
| Colon cancer | HT-29* | ++ |
| Colon cancer | KM-12* | +++ |
| Colon carcinoma | HCT 116 | +++ |
| Colon carcinoma | RKO | +++ |
| Diffuse large B-cell lymphoma | DB | +++ |
| Diffuse large B-cell lymphoma | HT | +++ |
| Diffuse large B-cell lymphoma | RL | +++ |
| Diffuse large B-cell lymphoma | SU-DHL-6 | +++ |
| Duodenal adenocarcinoma | HuTu 80 | +++ |
| Embryonal rhabdomyosarcoma | A-204 | +++ |
| Embryonal rhabdomyosarcoma | RD | +++ |
| Endometrial adenocarcinoma | AN3 CA | +++ |
| Endometrial adenocarcinoma | KLE | + |
| Endometrial adenosquamous | RL95-2 | +++ |
| carcinoma | | |
| Epithelioid sarcoma | VA-ES-BJ | +++ |
| Fibrosarcoma | HT-1080 | +++ |
| Gastric adenocarcinoma | Hs 746T | +++ |
| Gastric carcinoma | SNU-5 | ++ |
| Signet ring cell gastric | KATO III | ++ |
| adenocarcinoma | | |
| Gestational choriocarcinoma | JAR | +++ |
| Glioblastoma | A-172 | +++ |
| Glioblastoma | T98G | ++ |
| Glioblastoma | U-87 MG | +++ |
| Hereditary thyroid gland medullary | TT | ++ |
| carcinoma | | |
| Hypopharyngeal squamous cell | FaDu | +++ |
| carcinoma | | |
| Invasive ductal carcinoma | BT-20 | + |
| Invasive ductal carcinoma | BT-549 | +++ |
| Invasive ductal carcinoma | Hs 578T | +++ |
| Invasive ductal carcinoma | MCF7 | + |
| Liposarcoma | SW872 | +++ |
| Large cell lung carcinoma | NCI-H460 | +++ |
| Large cell lung carcinoma | NCI-H661 | ++ |
| Lung adenocarcinoma | A-427 | +++ |
| Lung adenocarcinoma | A-549 | + |
| Lung cancer | EKVX* | ++ |
| Lung cancer | HOP-62* | ++ |
| Lung cancer | HOP-92* | ++ |
| Lung cancer | NCI-H23* | ++ |
| Lung cancer | NCI-H322M* | ++ |
| Lung Cancer | NCI-H522* | +++ |
| Small cell lung carcinoma | NCI-H82 | +++ |
| Small cell lung carcinoma | SHP-77 | ++ |
| Squamous cell carcinoma | A388 | +++ |
| Squamous cell lung carcinoma | SW900 | ++ |
| Cutaneous melanoma | COLO 829 | +++ |
| Melanoma | G-361 | +++ |
| Melanoma | MeWo | ++ |
| Melanoma | RPMI-7951 | +++ |
| Melanoma | LOX IMVI* | +++ |
| Melanoma | M14* | +++ |
| Melanoma | MALME-3M* | +++ |
| Melanoma | MDA-MB- | +++ |
| | 435* | |
| Melanoma | SK-MEL-2* | +++ |
| Melanoma | SK-MEL-28* | +++ |
| Melanoma | SK-MEL-5* | +++ |
| Melanoma | UACC-62* | +++ |
| Melanoma | UACC-257* | ++ |
| Neuroblastoma | SK-N-AS | +++ |
| Neuroblastoma | SK-N-FI | +++ |
| Osteosarcoma | MG-63 | +++ |
| Osteosarcoma | U-2 OS | +++ |
| Ovarian | SK-OV-3* | +++ |
| Ovarian cancer | IGR-OV1* | +++ |
| Ovarian Cancer | OVCAR-4* | ++ |
| Ovarian Cancer | OVCAR-5* | +++ |
| Ovarian Cancer | OVCAR-8* | +++ |
| Ovarian clear cell adenocarcinoma | ES-2 | +++ |
| Ovarian mixed germ cell tumor | PA-1 | +++ |
| High grade ovarian serous | OVCAR-3 | ++ |
| adenocarcinoma | | |
| Pancreatic adenocarcinoma | Hs 766T | ++ |
| Pancreatic ductal adenocarcinoma | AsPC-1 | ++ |
| Pancreatic ductal adenocarcinoma | BxPC-3 | +++ |
| Pancreatic ductal adenocarcinoma | MIA PaCa-2 | ++ |
| Papillary renal cell carcinoma | ACHN | ++ |
| Primitive neuroectodermal tumor | PFSK-1 | +++ |
| Prostate carcinoma | LNCaP FGC | ++ |
| Prostate carcinoma | PC-3 | ++ |
| Prostate carcinoma | DU 145 | ++ |
| Rectal adenocarcinoma | SW837 | ++ |
| Medulloblastoma | Daoy | +++ |
| Renal cancer | CAKI-1* | + |
| Renal cancer | RXF-393* | + |
| Renal cancer | SN12C* | +++ |
| Renal cancer | TK-10* | ++ |
| Renal cancer | UO-31* | ++ |
| Renal cell carcinoma | 769-P | +++ |
| Renal cell carcinoma | 786-O | +++ |
| Renal cell carcinoma | A-498 | ++ |
| Renal cell carcinoma | A-704 | ++ |
| T acute lymphoblastic leukemia | CCRF-CEM | +++ |
| T acute lymphoblastic leukemia | Jurkat E6.1 | +++ |
| T acute lymphoblastic leukemia | MOLT-4 | +++ |
| T lymphoblastic lymphoma | SUP-T1 | +++ |
| Testicular embryonal carcinoma | NCCIT | +++ |
| Tongue squamous cell carcinoma | CAL 27 | +++ |

1. A compound represented by the structure of Formula IV: 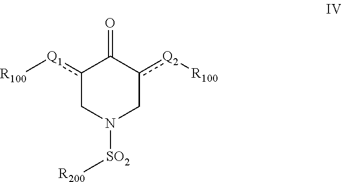
wherein
Q₁ and Q₂ are each independently, either CH or CH₂,
R₁₀₀ is selected from:
(i) phenyl, optionally substituted with 1-5 substituents (i.e., aryl) selected from the group consisting of: F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl;
(ii) naphthyl, optionally substituted with 1-5 substituents selected from the consisting of F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl;
(iii) a 5 or 6 membered monocyclic heteroaryl group, having 1-3 heteroatoms selected from the group consisting of O, N, and S, optionally substituted with 1-3 substituents selected from the group consisting of: F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl;
(iv) an 8 to 10 membered bicyclic heteroaryl group containing 1-3 heteroatoms selected from the group consisting of O, N, and S; and the second ring is fused to the first ring using 3 to 4 carbon atoms, and the bicyclic heteroaryl group is optionally substituted with 1-3 substituents selected from the group consisting of F, Cl, Br, I, OH, R₁₃, OR₁₃, SH, SR₁₃, R₁₅—OH, R₁₅—SH, —R₁₅—O—R₁₃, CF₃, OCF₃, CD₃, OCD₃, CN, NO₂, —R₁₅—CN, NH₂, NHR₁₃, N(R₁₃)₂, NR₁₃R₁₄, R₁₅—N(R₁₃)(R₁₄), R₁₆-R₁₅—N(R₁₃)(R₁₄), B(OH)₂, —OC(O)CF₃, —OCH₂Ph, NHC(O)—R₁₃, NR₁₃C(O)R₁₄, NR₁₃C(O)OR₁₄, NR₁₃SO₂R₁₄, NHCO—N(R₁₃)(R₁₄), COOH, —C(O)Ph, C(O)O—R₁₃, R₁₅—C(O)—R₁₃, C(O)H, C(O)—R₁₃, C₁-C₅ linear or branched C(O)-haloalkyl, —C(O)NH₂, C(O)NHR₁₃, C(O)N(R₁₃)(R₁₄), SO₂R₁₃, S(O)R₁₃, SO₂N(R₁₃)(R₁₄), CH(CF₃)(NH—R₁₃), C₁-C₁₄ linear or branched haloalkyl, C₁-C₁₄ linear, branched or cyclic alkyl, C₁-C₁₄ linear, branched or cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl; and
(v) a substituted or unsubstituted C₁-C₅ linear or branched alkyl or a substituted or unsubstituted C₁-C₅ linear or branched alkene wherein substitutions include at least one selected of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, aryl, phenyl, heteroaryl, OH, COOH, NH₂, N(R₁₃)(R₁₄), N₃, CF₃, CN or NO₂;
R₂₀₀ is amine (—NR₁₃R₁₄), OH, —OCOR₁₃, OR₁₃, substituted or unsubstituted linear or branched (C₂-C₁₄) alkyl, substituted or unsubstituted linear or branched (C₁-C₁₄) alkyl-NR₁₃R₁₄, substituted or unsubstituted linear or branched (C₁-C₁₄) alkyl-NHR₁₃, substituted or unsubstituted linear or branched (C₂-C₁₄) alkenyl-NR₁₃R₁₄, substituted or unsubstituted linear or branched (C₂-C₁₄) alkenyl-NHR₁₃, substituted or unsubstituted linear or branched (C₁-C₁₄) alkyl-OR₁₃, substituted or unsubstituted (C₃-C₈) cycloalkyl, R₁₅—N(R₁₃)(R₁₄), R₁₅—O(R₁₃), R₁₅—Cl, R₁₅—Br, R₁₅—F, R₁₅—I, R₁₅—N₃, R₁₅—CH═CH₂, and R₁₅—C≡CH; wherein substitutions include at least one selected of: F, Cl, Br, I, C₁-C₅ linear or branched alkyl, C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, OH, COOH, NH₂, N(R₁₃)(R₁₄), N₃, CF₃, CN or NO₂;
R₁₃ and R₁₄ are each independently selected from: H, Cl, Br, I, F, OH, substituted or unsubstituted C₁-C₁₄ linear or branched alkyl group, substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl (e.g. CH₂—C≡CH), aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₅ is [CH₂]ₚ
wherein p is between 1 and 10; and
R₁₆ is [CH]_{q}, [C]_{q}
wherein q is between 2 and 10;
or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof.

2. The compound of claim 1, wherein R₂₀₀ is R₁₅—N(R₁₃)(R₁₄), R₁₅—O(R₁₃), R₁₅—Cl, or R₁₅—Br;
R₁₅ is (CH₂)₂ or (CH₂)₃;
R₁₃ is CH₃;
R₁₄ is CH₃ or a C₁-C₁₄ linear alkyl group substituted with C₁-C₁₄ linear or branched alkynyl or N₃;
R₁₀₀ is substituted phenyl or a substituted 5 or 6 membered monocylclic heteroaryl, preferably wherein R₁₀₀ is substituted with at least one selected from: CH₃, F, Cl, NO₂, CF₃ or CN; or
R₁₀₀ is an aryl represented by the structure of formula V: 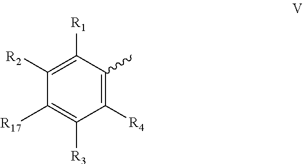
wherein
R₁, R₂, R₃, R₄ and R₁₇ are each independently selected from: H, NO₂, OH, COOK NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group, R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —N(R₁₃)(R₁₄), —CON(R₁₃)(R₁₄), N₃, S(O)R₁₃, and S(O)₂R₁₃;
R₁₃ and R₁₄ are each independently selected from: H, Cl, Br, I, F, OH, substituted or unsubstituted C₁-C₁₄ linear or branched alkyl group, substituted or unsubstituted (C₃-C₈) cycloalkyl, substituted or unsubstituted (C₃-C₈) heterocyclic ring having one or more heteroatoms selected from N, O and S; substituted or unsubstituted aryl (e.g., phenyl), substituted or unsubstituted heteroaryl (e.g., pyridyl), —C(O)—C₁-C₁₄ substituted or unsubstituted linear or branched alkyl (e.g., C(O)—CH₃), or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl (e.g. CH₂—C≡CH), aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₅ is [CH₂]ₚ
wherein p is between 1 and 10; and
R₁₆ is [CH]_{q}, [C]_{q}
wherein q is between 2 and 10.

3. (canceled)

4. (canceled)

5. The compound of claim 2, wherein R₁₇ is CN, Cl or F and R₂ is Cl, CF₃ or H.

6. (canceled)

7. (canceled)

8. The compound of claim 1 represented by the structure of the following compounds:

| Com- | |
|---|---|
| pound | |
| name | Structure |
| D1 | 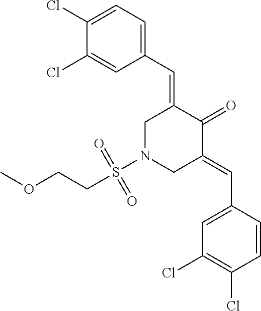 [SMILES: COCCS(=O)(=O)N1C/C(=C\c2ccc(Cl)c(Cl)c2)C(=O)/C(=C/c2ccc(Cl)c(Cl)c2)C1] |
| AA | 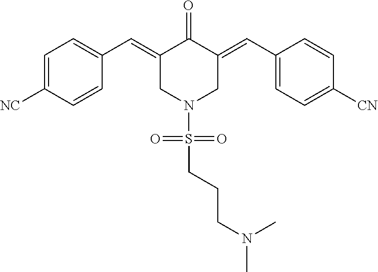 [SMILES: CN(C)CCCS(=O)(=O)N1C/C(=C\c2ccc(C#N)cc2)C(=O)/C(=C/c2ccc(C#N)cc2)C1] |
| CA | 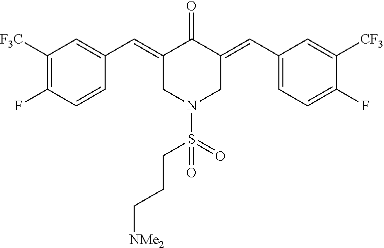 [SMILES: CN(C)CCCS(=O)(=O)N1C/C(=C\c2ccc(F)c(C(F)(F)F)c2)C(=O)/C(=C/c2ccc(F)c(C(F)(F)F)c2)C1] |
| E1 | 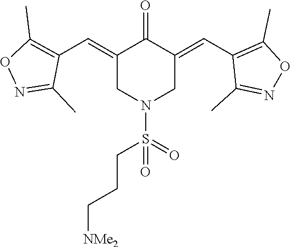 [SMILES: Cc1noc(C)c1/C=C1\CN(S(=O)(=O)CCCN(C)C)C/C(=C\c2c(C)noc2C)C1=O] |
| BA | 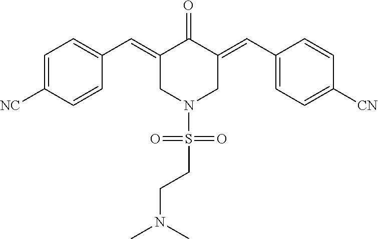 [SMILES: CN(C)CCS(=O)(=O)N1C/C(=C\c2ccc(C#N)cc2)C(=O)/C(=C/c2ccc(C#N)cc2)C1] |
| F1 | 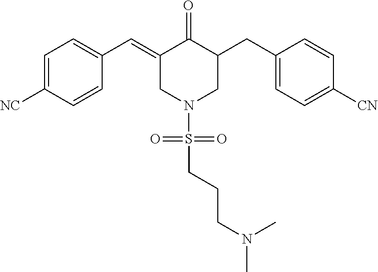 [SMILES: CN(C)CCCS(=O)(=O)N1C/C(=C\c2ccc(C#N)cc2)C(=O)C(Cc2ccc(C#N)cc2)C1] |
| A2 | 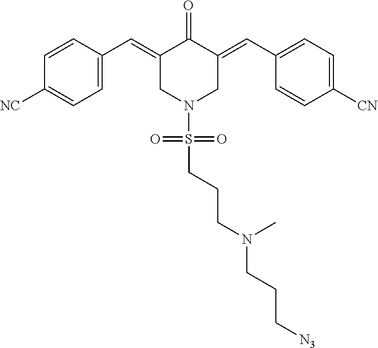 [SMILES: CN(CCCN=[N+]=[N-])CCCS(=O)(=O)N1C/C(=C\c2ccc(C#N)cc2)C(=O)/C(=C/c2ccc(C#N)cc2)C1] |
| BA-2 | 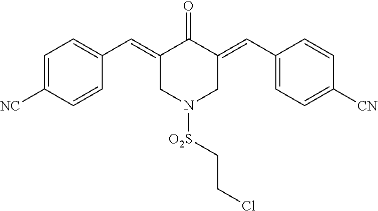 [SMILES: N#Cc1ccc(/C=C2\CN(S(=O)(=O)CCCl)C/C(=C\c3ccc(C#N)cc3)C2=O)cc1] |
| A3 | 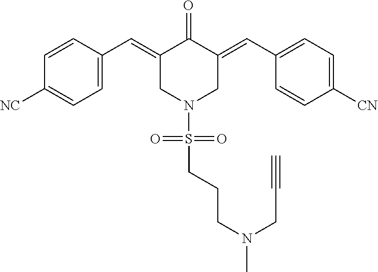 [SMILES: C#CCN(C)CCCS(=O)(=O)N1C/C(=C\c2ccc(C#N)cc2)C(=O)/C(=C/c2ccc(C#N)cc2)C1] |
| CA-2 | 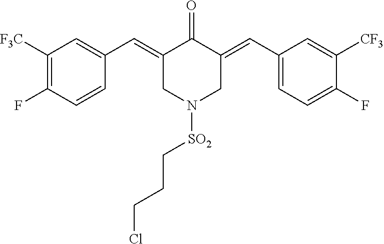 [SMILES: O=C1/C(=C/c2ccc(F)c(C(F)(F)F)c2)CN(S(=O)(=O)CCCCl)C/C1=C\c1ccc(F)c(C(F)(F)F)c1] |
| F1-5 | 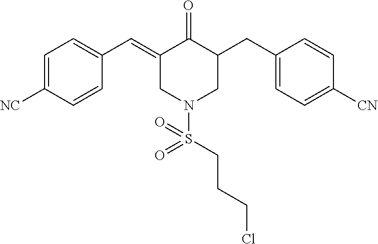 [SMILES: N#Cc1ccc(/C=C2\CN(S(=O)(=O)CCCCl)CC(Cc3ccc(C#N)cc3)C2=O)cc1] |
| E1-2 | 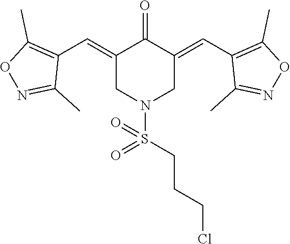 [SMILES: Cc1noc(C)c1/C=C1\CN(S(=O)(=O)CCCCl)C/C(=C\c2c(C)noc2C)C1=O] |
| AA-8 | 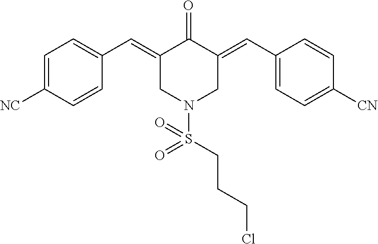 [SMILES: N#Cc1ccc(/C=C2\CN(S(=O)(=O)CCCCl)C/C(=C\c3ccc(C#N)cc3)C2=O)cc1] |

or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof.

9. A compound represented by the structure of Formula III: 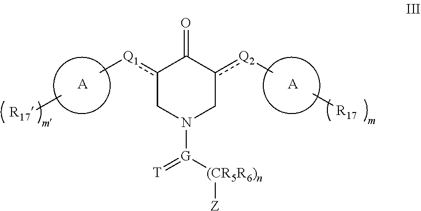
wherein
A ring is a single or fused aromatic or heteroaromatic ring system, or a single or fused C₃-C₁₀ cycloalkyl, or a single or fused C₃-C₁₀ heterocyclic ring;
Q₁ and Q₂ are each independently, either CH or CH₂;
R₅ and R₆ are each, independently, selected from: H, F, Cl, Br, I, OH, R₁₅—OH, COOH, CN, unsubstituted C₁-C₁₀ alkyl, OR₁₃, N(R₁₃)(R₁₄), substituted or unsubstituted (C₃—C₈) cycloalkyl, substituted or unsubstituted (C₃-C₈) heterocyclic ring having one or more heteroatoms selected from N, O and S;
or R₅ and R₆ are joint to form a substituted or unsubstituted (C₃-C₈) cycloalkyl or an unsubstituted (C₃-C₈) heterocyclic ring; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₇ and R₈ are each independently selected from: H, F, Cl, Br, I, substituted or unsubstituted linear or branched C₁-C₁₀ alkyl, substituted or unsubstituted linear or branched C₁-C₁₀ alkoxy, substituted or unsubstituted aryl, substituted or unsubstituted heteroaryl, C(O)—R₁₃, S(O)—R₁₃, S(O)₂—R₁₃, R₁₅-Ph, R₁₅-aryl, R₁₅-heteroaryl, R₁₅-R₁₃, R₁₅R₁₆-R₁₃, —CH₂—CH═CH—C₁-C₁₀ alkyl, —CH₂—CH═CH₂, substituted or unsubstituted (C₃-C₈) cycloalkyl, substituted or unsubstituted (C₃-C₈) heterocyclic ring having one or more heteroatoms selected from N, O and S; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q1}R₁₃;
R₁₃ is selected from: H, Cl, Br, I, F, OH, substituted or unsubstituted C₁-C₁₄ linear or branched alkyl group, substituted or unsubstituted (C₃-C₈) cycloalkyl, substituted or unsubstituted (C₃-C₈) heterocyclic ring having one or more heteroatoms selected from N, O and S; substituted or unsubstituted aryl, substituted or unsubstituted heteroaryl, —C(O)—C₁-C₁₄ substituted or unsubstituted linear or branched alkyl, or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₄ is selected from: Cl, Br, I, F, OH, substituted or unsubstituted C₁-C₁₄ linear or branched alkyl group, substituted or unsubstituted (C₃-C₈) cycloalkyl, substituted or unsubstituted (C₃-C₈) heterocyclic ring having one or more heteroatoms selected from N, O and S; substituted or unsubstituted aryl, substituted or unsubstituted heteroaryl, —C(O)—C₁-C₁₄ substituted or unsubstituted linear or branched alkyl, or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₅ is [CH₂]p
wherein p is between 1 and 10;
R₁₆ is [CH]_{q}, [C]_{q}
wherein q is between 2 and 10;
n is an integer between 1 and 15;
R₁₇ and R₁₇′ are each independently selected from H, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group, R₁₅—C00R₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
m and m′ are each independently an integer between 0 and 5;
G is C, S or N;
T is O, S, NH, N—OH, CH₂, or CR₁₃R₁₄; or
G=T is SO₂
Z is —NH—C(O)—R₁₅—N(R₇)(R₈), F, Br, I, N(R₁₃)(R₁₄), OR₁₃, —NH—C(O)—R₁₅-R₁₃, substituted or unsubstituted aryl, substituted or unsubstituted heteroaryl, substituted or unsubstituted R₁₅-aryl, substituted or unsubstituted R₁₅-heteroaryl, or C(O)—NH—R₁₃;
or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof.

10. The compound of claim 9, wherein A is a phenyl or an isoxazole; m and m′ are each independently 1 or 2, and R₁₇ and R₁₇′ are each independently H, F, Cl, Br, I, CN, CH₃ or CF₃; O₁ is CH; O₂ is CH or CH₂; R₅ and R₆ are each independently H, OH, R₁₅—OH, CH₂—OH, COOH, C₁-C₁₀ alkyl, iPr, OR₁₃, OMe, NH₂, N(R₁₃)(R₁₄), N(CH₃)₂, or R and R₆ are joined to form a substituted or unsubstituted (C₃-C₈) cycloalkyl, a cyclopropyl, a substituted or unsubstituted (C₃-C₈) heterocyclic ring, or a morpholine; G is C, T is O, or G=T is SO₂, R₁₃ is H, OH, methyl, methoxyethyl, phenyl, pyridyl, or C(O)—CH₃; R₄ is H, or methyl; R₇ is a methyl, C₃ alkyl substituted with N₃, a propyl-azide or CH₂—C≡CH; R₈ is a methyl; or any combination thereof.

11.-16. (canceled)

17. The compound of claim 9, represented by the following structures:

| Compound name | Structure |
|---|---|
| B9 | 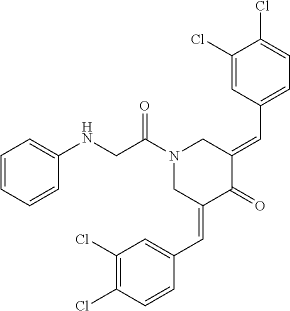 [SMILES: O=C1/C(=C/c2ccc(Cl)c(Cl)c2)CN(C(=O)CNc2ccccc2)C/C1=C\c1ccc(Cl)c(Cl)c1] |
| B10 | 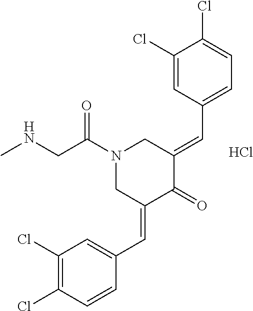 [SMILES: CNCC(=O)N1C/C(=C\c2ccc(Cl)c(Cl)c2)C(=O)/C(=C/c2ccc(Cl)c(Cl)c2)C1 ; Cl] |
| B11 | 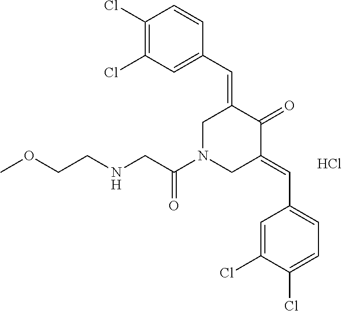 [SMILES: COCCNCC(=O)N1C/C(=C\c2ccc(Cl)c(Cl)c2)C(=O)/C(=C/c2ccc(Cl)c(Cl)c2)C1 ; Cl] |
| B12 | 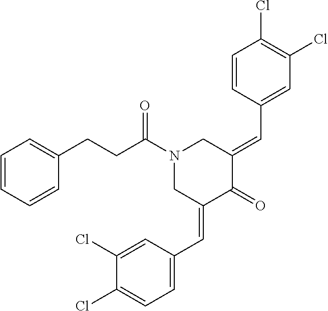 [SMILES: O=C1/C(=C/c2ccc(Cl)c(Cl)c2)CN(C(=O)CCc2ccccc2)C/C1=C\c1ccc(Cl)c(Cl)c1] |
| B13 | 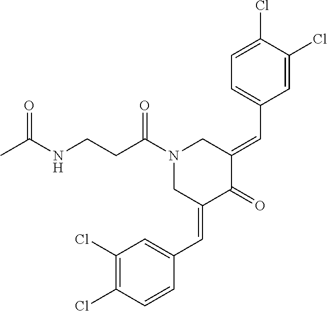 [SMILES: CC(=O)NCCC(=O)N1C/C(=C\c2ccc(Cl)c(Cl)c2)C(=O)/C(=C/c2ccc(Cl)c(Cl)c2)C1] |
| B14 | 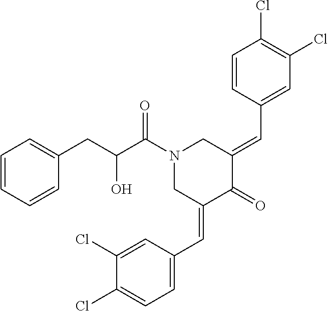 [SMILES: O=C1/C(=C/c2ccc(Cl)c(Cl)c2)CN(C(=O)C(O)Cc2ccccc2)C/C1=C\c1ccc(Cl)c(Cl)c1] |
| B15 | 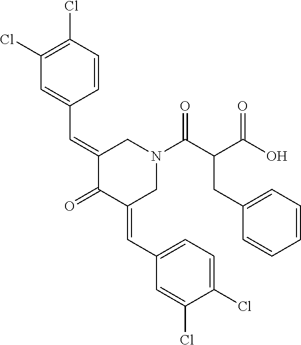 [SMILES: O=C1/C(=C/c2ccc(Cl)c(Cl)c2)CN(C(=O)C(Cc2ccccc2)C(=O)O)C/C1=C\c1ccc(Cl)c(Cl)c1] |
| B16 | 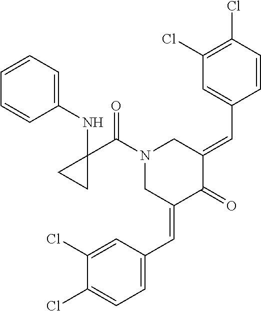 [SMILES: O=C1/C(=C/c2ccc(Cl)c(Cl)c2)CN(C(=O)C2(Nc3ccccc3)CC2)C/C1=C\c1ccc(Cl)c(Cl)c1] |
| B17 | 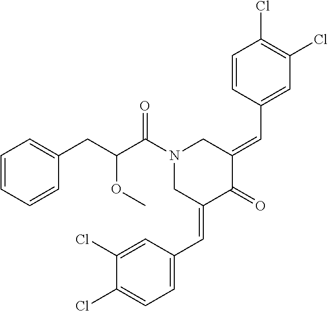 [SMILES: COC(Cc1ccccc1)C(=O)N1C/C(=C\c2ccc(Cl)c(Cl)c2)C(=O)/C(=C/c2ccc(Cl)c(Cl)c2)C1] |
| | 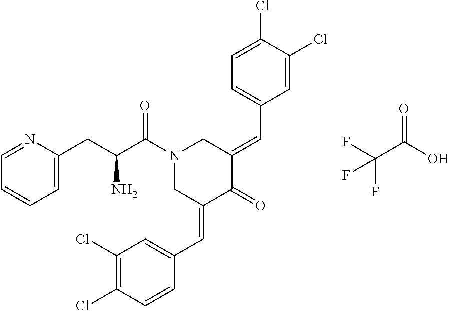 [SMILES: N[C@@H](Cc1ccccn1)C(=O)N1C/C(=C\c2ccc(Cl)c(Cl)c2)C(=O)/C(=C/c2ccc(Cl)c(Cl)c2)C1 ; O=C(O)C(F)(F)F] |
| | 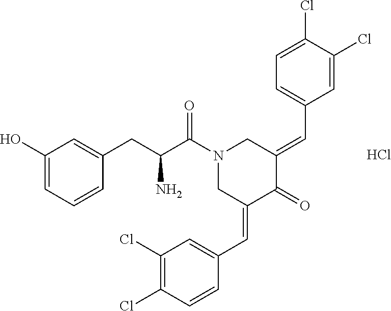 [SMILES: N[C@@H](Cc1cccc(O)c1)C(=O)N1C/C(=C\c2ccc(Cl)c(Cl)c2)C(=O)/C(=C/c2ccc(Cl)c(Cl)c2)C1 ; Cl] |
| | 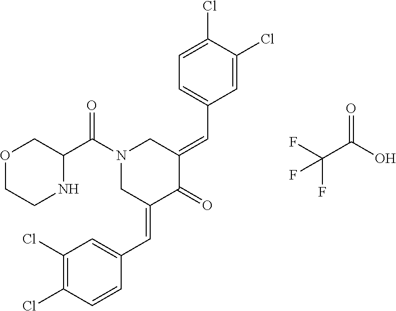 [SMILES: O=C1/C(=C/c2ccc(Cl)c(Cl)c2)CN(C(=O)C2COCCN2)C/C1=C\c1ccc(Cl)c(Cl)c1 ; O=C(O)C(F)(F)F] |
| | 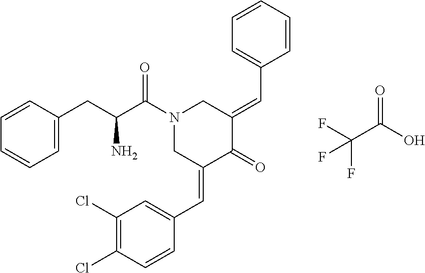 [SMILES: N[C@@H](Cc1ccccc1)C(=O)N1C/C(=C\c2ccccc2)C(=O)/C(=C/c2ccc(Cl)c(Cl)c2)C1 ; O=C(O)C(F)(F)F] |
| | 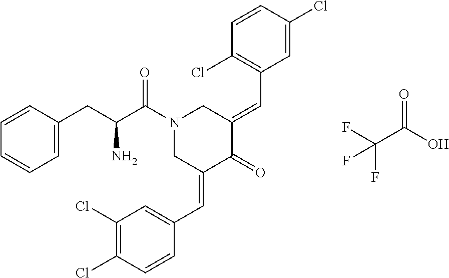 [SMILES: N[C@@H](Cc1ccccc1)C(=O)N1C/C(=C\c2ccc(Cl)c(Cl)c2)C(=O)/C(=C/c2cc(Cl)ccc2Cl)C1 ; O=C(O)C(F)(F)F] |
| B23 | 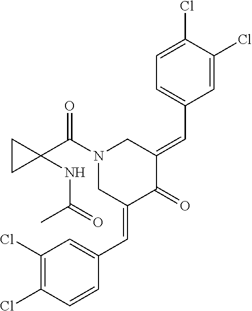 [SMILES: CC(=O)NC1(C(=O)N2C/C(=C\c3ccc(Cl)c(Cl)c3)C(=O)/C(=C/c3ccc(Cl)c(Cl)c3)C2)CC1] |
| B24 | 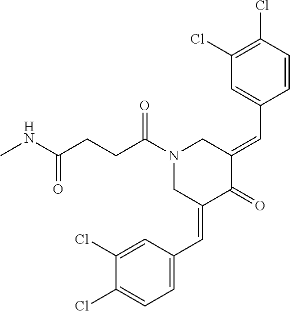 [SMILES: CNC(=O)CCC(=O)N1C/C(=C\c2ccc(Cl)c(Cl)c2)C(=O)/C(=C/c2ccc(Cl)c(Cl)c2)C1] |
| B25 | 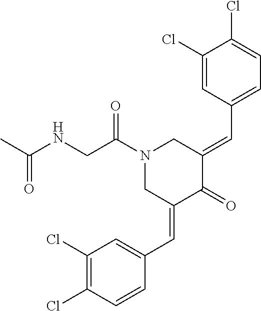 [SMILES: CC(=O)NCC(=O)N1C/C(=C\c2ccc(Cl)c(Cl)c2)C(=O)/C(=C/c2ccc(Cl)c(Cl)c2)C1] |
| B26 | 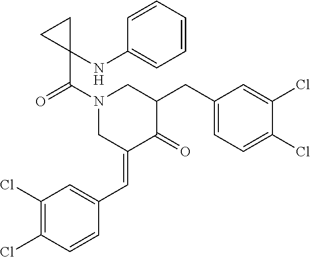 [SMILES: O=C1/C(=C/c2ccc(Cl)c(Cl)c2)CN(C(=O)C2(Nc3ccccc3)CC2)CC1Cc1ccc(Cl)c(Cl)c1] |
| B27 | 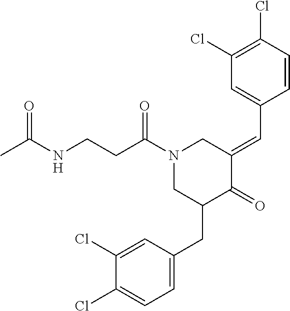 [SMILES: CC(=O)NCCC(=O)N1C/C(=C\c2ccc(Cl)c(Cl)c2)C(=O)C(Cc2ccc(Cl)c(Cl)c2)C1] |
| B28 | 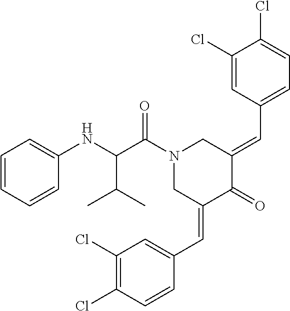 [SMILES: CC(C)C(Nc1ccccc1)C(=O)N1C/C(=C\c2ccc(Cl)c(Cl)c2)C(=O)/C(=C/c2ccc(Cl)c(Cl)c2)C1] |
| B29 | 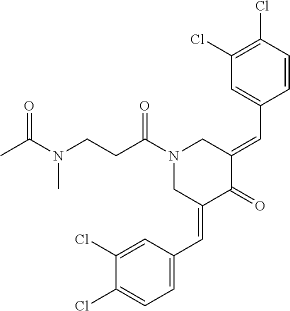 [SMILES: CC(=O)N(C)CCC(=O)N1C/C(=C\c2ccc(Cl)c(Cl)c2)C(=O)/C(=C/c2ccc(Cl)c(Cl)c2)C1] |
| B30 | 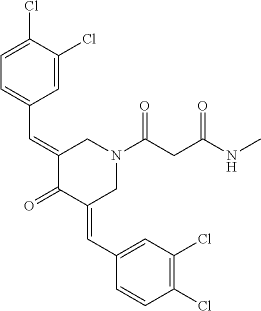 [SMILES: CNC(=O)CC(=O)N1C/C(=C\c2ccc(Cl)c(Cl)c2)C(=O)/C(=C/c2ccc(Cl)c(Cl)c2)C1] |
| B32 | 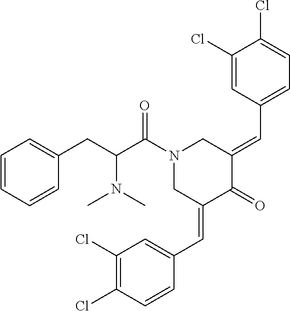 [SMILES: CN(C)C(Cc1ccccc1)C(=O)N1C/C(=C\c2ccc(Cl)c(Cl)c2)C(=O)/C(=C/c2ccc(Cl)c(Cl)c2)C1] |
| D1 | 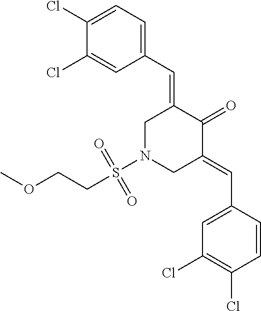 [SMILES: COCCS(=O)(=O)N1C/C(=C\c2ccc(Cl)c(Cl)c2)C(=O)/C(=C/c2ccc(Cl)c(Cl)c2)C1] |
| B6 | 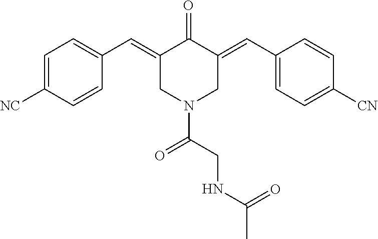 [SMILES: CC(=O)NCC(=O)N1C/C(=C\c2ccc(C#N)cc2)C(=O)/C(=C/c2ccc(C#N)cc2)C1] |
| B7 | 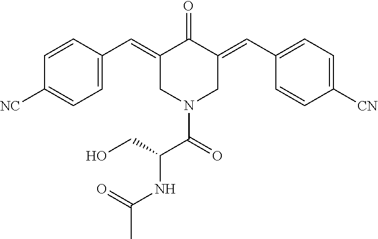 [SMILES: CC(=O)N[C@H](CO)C(=O)N1C/C(=C\c2ccc(C#N)cc2)C(=O)/C(=C/c2ccc(C#N)cc2)C1] |
| B1 | 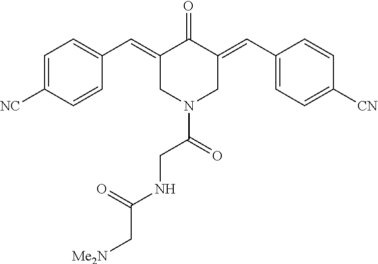 [SMILES: CN(C)CC(=O)NCC(=O)N1C/C(=C\c2ccc(C#N)cc2)C(=O)/C(=C/c2ccc(C#N)cc2)C1] |
| B8 | 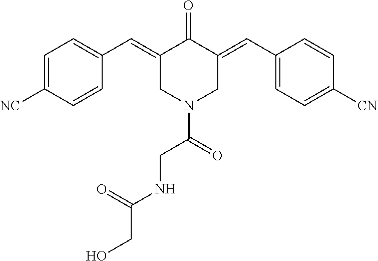 [SMILES: N#Cc1ccc(/C=C2\CN(C(=O)CNC(=O)CO)C/C(=C\c3ccc(C#N)cc3)C2=O)cc1] |
| AA | 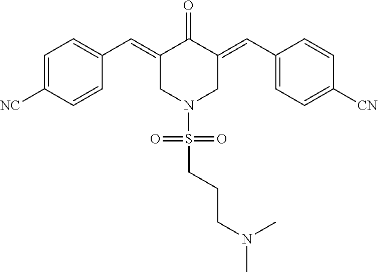 [SMILES: CN(C)CCCS(=O)(=O)N1C/C(=C\c2ccc(C#N)cc2)C(=O)/C(=C/c2ccc(C#N)cc2)C1] |
| E1 | 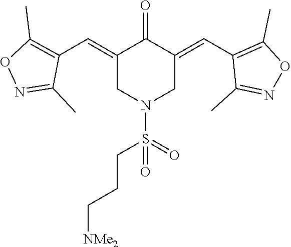 [SMILES: Cc1noc(C)c1/C=C1\CN(S(=O)(=O)CCCN(C)C)C/C(=C\c2c(C)noc2C)C1=O] |
| BA | 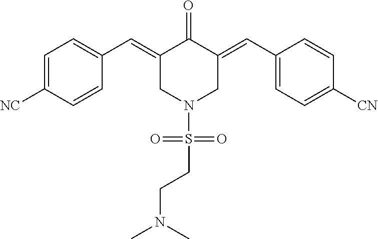 [SMILES: CN(C)CCS(=O)(=O)N1C/C(=C\c2ccc(C#N)cc2)C(=O)/C(=C/c2ccc(C#N)cc2)C1] |
| B3 | 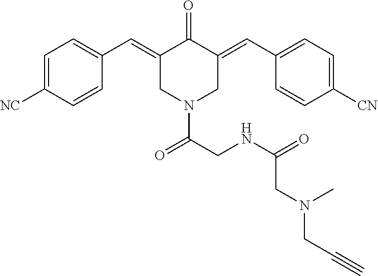 [SMILES: C#CCN(C)CC(=O)NCC(=O)N1C/C(=C\c2ccc(C#N)cc2)C(=O)/C(=C/c2ccc(C#N)cc2)C1] |
| B2 | 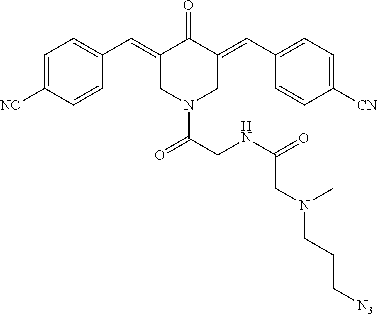 [SMILES: CN(CCCN=[N+]=[N-])CC(=O)NCC(=O)N1C/C(=C\c2ccc(C#N)cc2)C(=O)/C(=C/c2ccc(C#N)cc2)C1] |
| C1 | 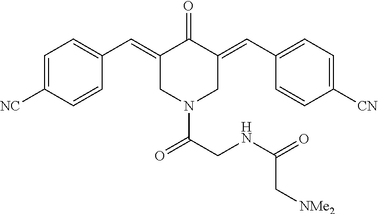 [SMILES: CN(C)CC(=O)NCC(=O)N1C/C(=C\c2ccc(C#N)cc2)C(=O)/C(=C/c2ccc(C#N)cc2)C1] |
| F1 | 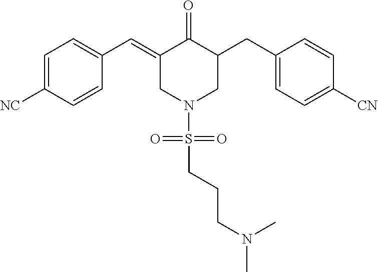 [SMILES: CN(C)CCCS(=O)(=O)N1C/C(=C\c2ccc(C#N)cc2)C(=O)C(Cc2ccc(C#N)cc2)C1] |
| H1 | 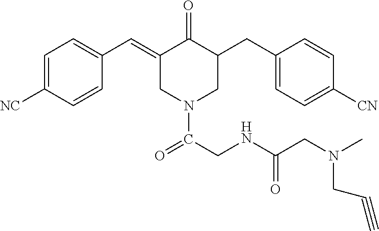 [SMILES: C#CCN(C)CC(=O)NCC(=O)N1C/C(=C\c2ccc(C#N)cc2)C(=O)C(Cc2ccc(C#N)cc2)C1] |
| B1-11 | 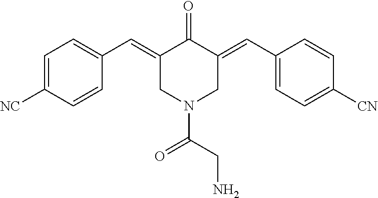 [SMILES: N#Cc1ccc(/C=C2\CN(C(=O)CN)C/C(=C\c3ccc(C#N)cc3)C2=O)cc1] |
| C1-7 | 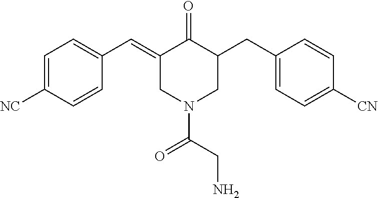 [SMILES: N#Cc1ccc(/C=C2\CN(C(=O)CN)CC(Cc3ccc(C#N)cc3)C2=O)cc1] |
| C1-8 | 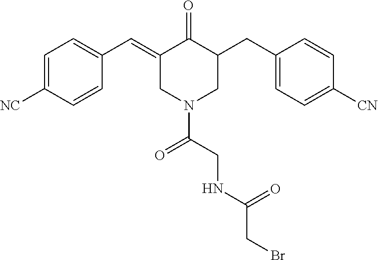 [SMILES: N#Cc1ccc(/C=C2\CN(C(=O)CNC(=O)CBr)CC(Cc3ccc(C#N)cc3)C2=O)cc1] |
| B2-7 | 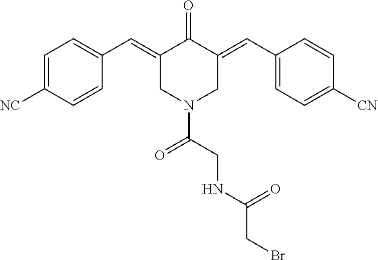 [SMILES: N#Cc1ccc(/C=C2\CN(C(=O)CNC(=O)CBr)C/C(=C\c3ccc(C#N)cc3)C2=O)cc1] |

or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof.

18. A compound, represented by the structure of Formula II: 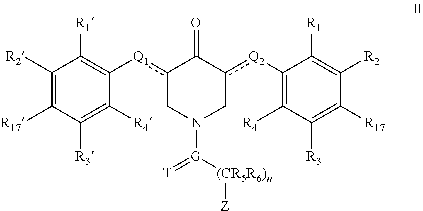
wherein
Q₁ and Q₂ are each independently, either CH or CH₂;
R₁, R₂, R₃, R₄R₁ ′, R₂′, R₃′, and R₄′ are each, independently, selected from:
H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group (e.g., methyl), R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₅ and R₆ are each, independently, selected from: H, F, Cl, Br, I, OH, R₁₅—OH, COOH, CN, unsubstituted C₁-C₁₀ alkyl, OR₁₃, N(R₁₃)(R₁₄), substituted or unsubstituted (C₃-C₈) cycloalkyl, substituted or unsubstituted (C₃-C₈) heterocyclic ring having one or more heteroatoms selected from N, O and S;
or R₅ and R₆ are joined to form a substituted or unsubstituted (C₃-C₈) cycloalkyl or an unsubstituted (C₃-C₈) heterocyclic ring; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₇ and R₈ are each independently selected from: H, F, Cl, Br, I, substituted or unsubstituted linear or branched C₁-C₁₀ alkyl, substituted or unsubstituted linear or branched C₁-C₁₀ alkoxy, substituted or unsubstituted aryl, substituted or unsubstituted heteroaryl, C(O)—R₁₃, S(O)—R₁₃, S(O)₂—R₁₃, R₁₅-Ph, R₁₅-aryl, R₁₅-heteroaryl, R₁₅-R₁₃, R₁₅-R₁₆-R₁₃, —CH₂—CH═CH—C₁-C₁₀ alkyl, —CH₂—CH═CH₂, substituted or unsubstituted (C₃-C₈) cycloalkyl, substituted or unsubstituted (C₃-C₈) heterocyclic ring having one or more heteroatoms selected from N, O and S; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q1}R₁₃;
R₁₃ is selected from: H, Cl, Br, I, F, OH, substituted or unsubstituted C₁-C₁₄ linear or branched alkyl group, substituted or unsubstituted (C₃-C₈) cycloalkyl, substituted or unsubstituted (C₃-C₈) heterocyclic ring having one or more heteroatoms selected from N, O and S; substituted or unsubstituted aryl, substituted or unsubstituted heteroaryl, —C(O)—C₁-C₁₄ substituted or unsubstituted linear or branched alkyl, or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₄ is selected from: Cl, Br, I, F, OH, substituted or unsubstituted C₁-C₁₄ linear or branched alkyl group, substituted or unsubstituted (C₃-C₈) cycloalkyl, substituted or unsubstituted (C₃-C₈) heterocyclic ring having one or more heteroatoms selected from N, O and S; substituted or unsubstituted aryl, substituted or unsubstituted heteroaryl, —C(O)—C₁-C₁₄ substituted or unsubstituted linear or branched alkyl, or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₅ is [CH₂]ₚ
wherein p is between 1 and 10;
R₁₆ is [CH]_{q}, [C]_{q}
wherein q is between 2 and 10;
n is an integer between 1 and 15;
R₁₇ and R₁₇′ are each independently selected from H, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group, R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
G is C, S or N;
T is O, S, NH, N—OH, CH₂, CR₁₃R₁₄; or
G=T is SO₂; and
Z is —NH—C(O)—R₁₅—N(R₇)(R₈), F, Cl, Br, I, N(R₁₃)(R₁₄), OR₁₃, —NH—C(O)—R₅-R₁₃, substituted or unsubstituted aryl, substituted or unsubstituted heteroaryl, substituted or unsubstituted R₁₅-aryl, substituted or unsubstituted R₁₅-heteroaryl, C(O)—NH—R₁₃;
or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof.

19. The compound of claim 18, wherein R₁₇ and R₁₇′ are each independently Cl, CN, H, F; R₂ and R₂′ are each independently H, CF₃, CN, Cl, NO₂; R₄ and R₄′ are each independently H or Cl; G is C, T is O, or G=T is SO₂; R₁₃ is H, OH, methyl, methoxyethyl, phenyl, pyridyl, or C(O)—CH₃; R₁₄ is methyl; R₇ is a methyl, C₃ alkyl substituted with N₃, propyl-azide or CH₂—C≡CH; R₈ is a methyl; or any combination thereof.

20.-22. (canceled)

23. The compound of claim 18, represented by the following structures:
Compound name Structure

| Compound name | Structure |
|---|---|
| B9 | 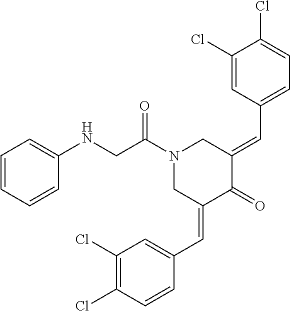 [SMILES: O=C1/C(=C/c2ccc(Cl)c(Cl)c2)CN(C(=O)CNc2ccccc2)C/C1=C\c1ccc(Cl)c(Cl)c1] |
| B10 | 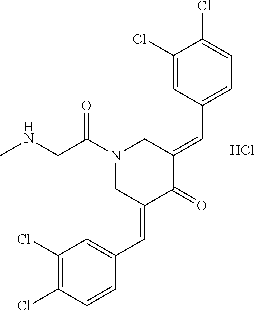 [SMILES: CNCC(=O)N1C/C(=C\c2ccc(Cl)c(Cl)c2)C(=O)/C(=C/c2ccc(Cl)c(Cl)c2)C1 ; Cl] |
| B11 | 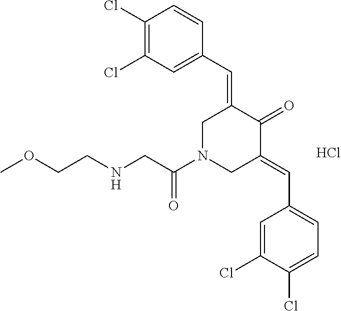 [SMILES: COCCNCC(=O)N1C/C(=C\c2ccc(Cl)c(Cl)c2)C(=O)/C(=C/c2ccc(Cl)c(Cl)c2)C1 ; Cl] |
| B12 | 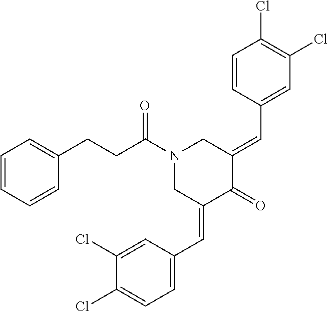 [SMILES: O=C1/C(=C/c2ccc(Cl)c(Cl)c2)CN(C(=O)CCc2ccccc2)C/C1=C\c1ccc(Cl)c(Cl)c1] |
| B13 | 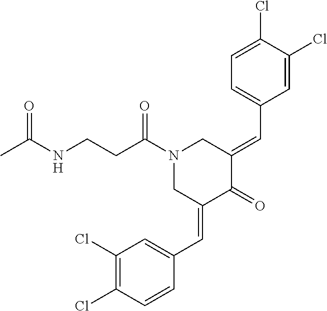 [SMILES: CC(=O)NCCC(=O)N1C/C(=C\c2ccc(Cl)c(Cl)c2)C(=O)/C(=C/c2ccc(Cl)c(Cl)c2)C1] |
| B14 | 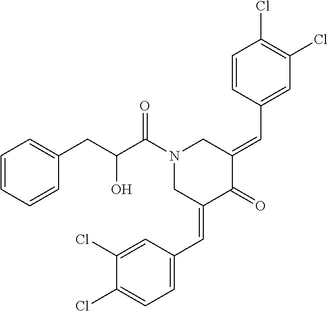 [SMILES: O=C1/C(=C/c2ccc(Cl)c(Cl)c2)CN(C(=O)C(O)Cc2ccccc2)C/C1=C\c1ccc(Cl)c(Cl)c1] |
| B15 | 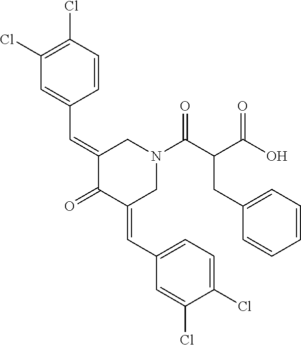 [SMILES: O=C1/C(=C/c2ccc(Cl)c(Cl)c2)CN(C(=O)C(Cc2ccccc2)C(=O)O)C/C1=C\c1ccc(Cl)c(Cl)c1] |
| B16 | 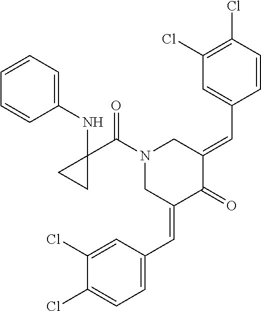 [SMILES: O=C1/C(=C/c2ccc(Cl)c(Cl)c2)CN(C(=O)C2(Nc3ccccc3)CC2)C/C1=C\c1ccc(Cl)c(Cl)c1] |
| B17 | 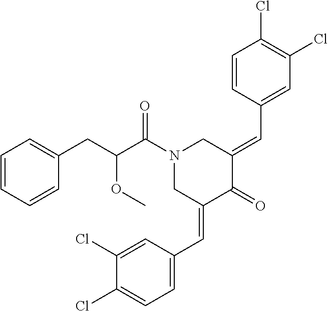 [SMILES: COC(Cc1ccccc1)C(=O)N1C/C(=C\c2ccc(Cl)c(Cl)c2)C(=O)/C(=C/c2ccc(Cl)c(Cl)c2)C1] |
| | 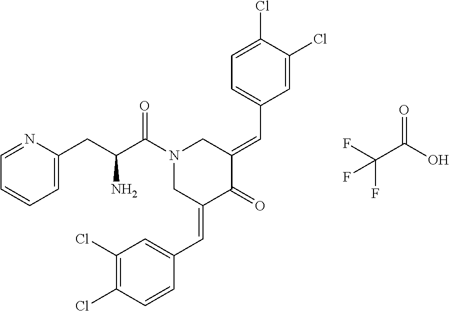 [SMILES: N[C@@H](Cc1ccccn1)C(=O)N1C/C(=C\c2ccc(Cl)c(Cl)c2)C(=O)/C(=C/c2ccc(Cl)c(Cl)c2)C1 ; O=C(O)C(F)(F)F] |
| | 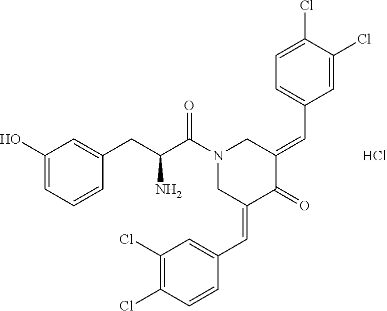 [SMILES: N[C@@H](Cc1cccc(O)c1)C(=O)N1C/C(=C\c2ccc(Cl)c(Cl)c2)C(=O)/C(=C/c2ccc(Cl)c(Cl)c2)C1 ; Cl] |
| | 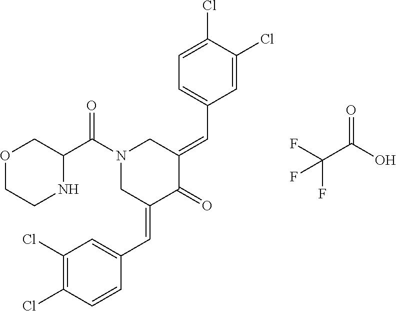 [SMILES: O=C1/C(=C/c2ccc(Cl)c(Cl)c2)CN(C(=O)C2COCCN2)C/C1=C\c1ccc(Cl)c(Cl)c1 ; O=C(O)C(F)(F)F] |
| | 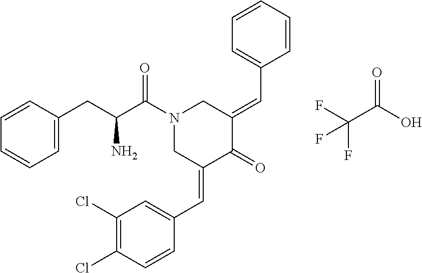 [SMILES: N[C@@H](Cc1ccccc1)C(=O)N1C/C(=C\c2ccccc2)C(=O)/C(=C/c2ccc(Cl)c(Cl)c2)C1 ; O=C(O)C(F)(F)F] |
| | 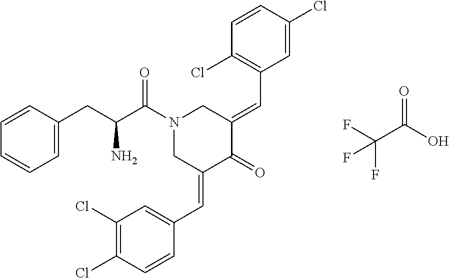 [SMILES: N[C@@H](Cc1ccccc1)C(=O)N1C/C(=C\c2ccc(Cl)c(Cl)c2)C(=O)/C(=C/c2cc(Cl)ccc2Cl)C1 ; O=C(O)C(F)(F)F] |
| B23 | 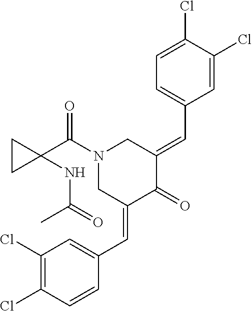 [SMILES: CC(=O)NC1(C(=O)N2C/C(=C\c3ccc(Cl)c(Cl)c3)C(=O)/C(=C/c3ccc(Cl)c(Cl)c3)C2)CC1] |
| B24 | 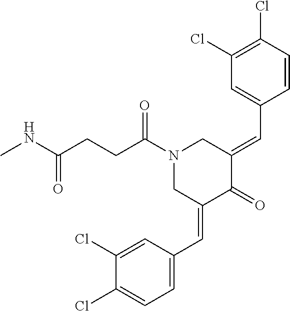 [SMILES: CNC(=O)CCC(=O)N1C/C(=C\c2ccc(Cl)c(Cl)c2)C(=O)/C(=C/c2ccc(Cl)c(Cl)c2)C1] |
| B25 | 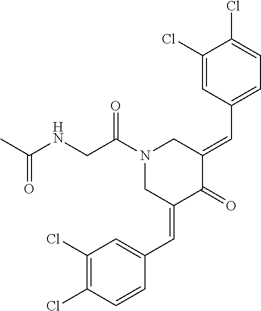 [SMILES: CC(=O)NCC(=O)N1C/C(=C\c2ccc(Cl)c(Cl)c2)C(=O)/C(=C/c2ccc(Cl)c(Cl)c2)C1] |
| B26 | 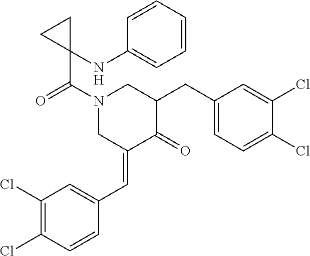 [SMILES: O=C1/C(=C/c2ccc(Cl)c(Cl)c2)CN(C(=O)C2(Nc3ccccc3)CC2)CC1Cc1ccc(Cl)c(Cl)c1] |
| B27 | 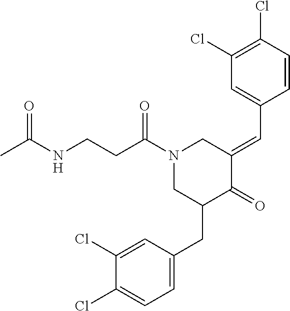 [SMILES: CC(=O)NCCC(=O)N1C/C(=C\c2ccc(Cl)c(Cl)c2)C(=O)C(Cc2ccc(Cl)c(Cl)c2)C1] |
| B28 | 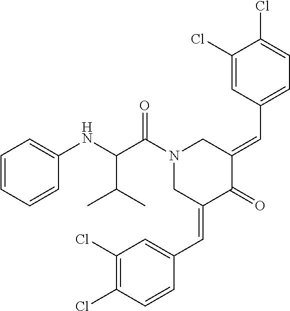 [SMILES: CC(C)C(Nc1ccccc1)C(=O)N1C/C(=C\c2ccc(Cl)c(Cl)c2)C(=O)/C(=C/c2ccc(Cl)c(Cl)c2)C1] |
| B29 | 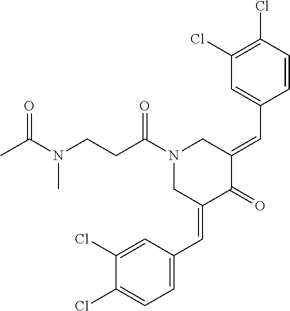 [SMILES: CC(=O)N(C)CCC(=O)N1C/C(=C\c2ccc(Cl)c(Cl)c2)C(=O)/C(=C/c2ccc(Cl)c(Cl)c2)C1] |
| B30 | 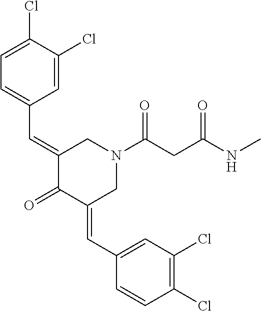 [SMILES: CNC(=O)CC(=O)N1C/C(=C\c2ccc(Cl)c(Cl)c2)C(=O)/C(=C/c2ccc(Cl)c(Cl)c2)C1] |
| | 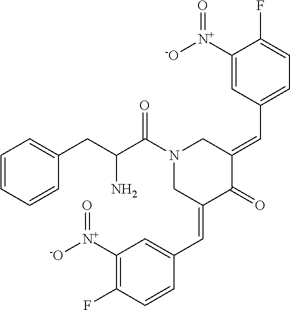 |
| B4 | 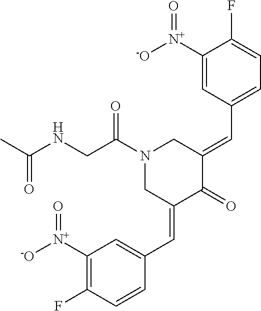 |
| B32 | 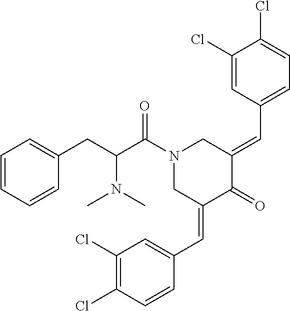 [SMILES: CN(C)C(Cc1ccccc1)C(=O)N1C/C(=C\c2ccc(Cl)c(Cl)c2)C(=O)/C(=C/c2ccc(Cl)c(Cl)c2)C1] |
| D1 | 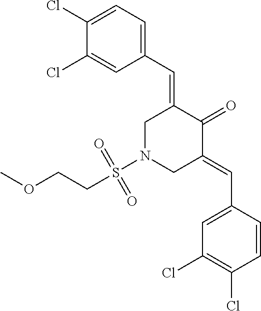 [SMILES: COCCS(=O)(=O)N1C/C(=C\c2ccc(Cl)c(Cl)c2)C(=O)/C(=C/c2ccc(Cl)c(Cl)c2)C1] |
| B5 | 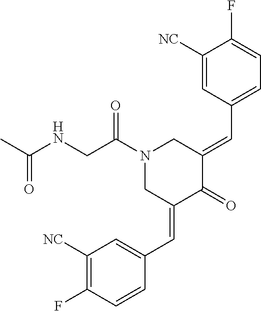 [SMILES: CC(=O)NCC(=O)N1C/C(=C\c2ccc(F)c(C#N)c2)C(=O)/C(=C/c2ccc(F)c(C#N)c2)C1] |
| B6 | 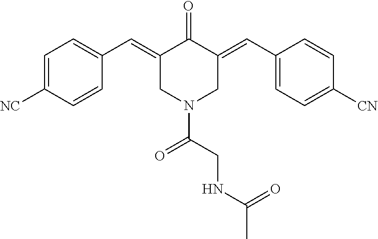 [SMILES: CC(=O)NCC(=O)N1C/C(=C\c2ccc(C#N)cc2)C(=O)/C(=C/c2ccc(C#N)cc2)C1] |
| B7 | 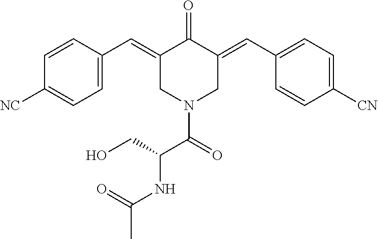 [SMILES: CC(=O)N[C@H](CO)C(=O)N1C/C(=C\c2ccc(C#N)cc2)C(=O)/C(=C/c2ccc(C#N)cc2)C1] |
| B1 | 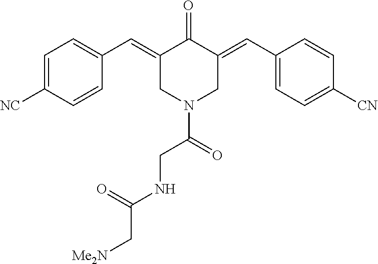 [SMILES: CN(C)CC(=O)NCC(=O)N1C/C(=C\c2ccc(C#N)cc2)C(=O)/C(=C/c2ccc(C#N)cc2)C1] |
| B8 | 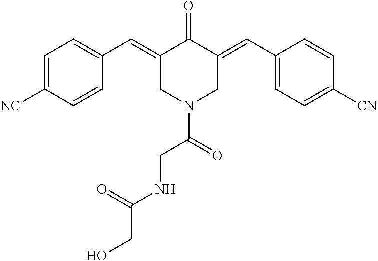 [SMILES: N#Cc1ccc(/C=C2\CN(C(=O)CNC(=O)CO)C/C(=C\c3ccc(C#N)cc3)C2=O)cc1] |
| AA | 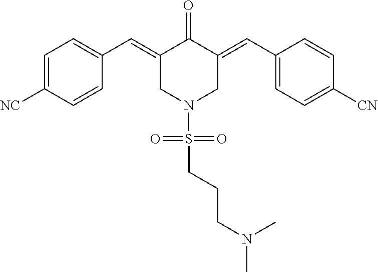 [SMILES: CN(C)CCCS(=O)(=O)N1C/C(=C\c2ccc(C#N)cc2)C(=O)/C(=C/c2ccc(C#N)cc2)C1] |
| CA | 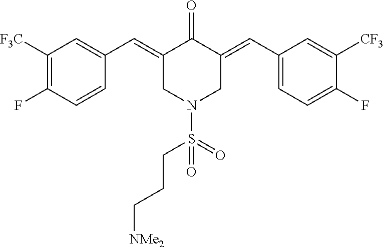 [SMILES: CN(C)CCCS(=O)(=O)N1C/C(=C\c2ccc(F)c(C(F)(F)F)c2)C(=O)/C(=C/c2ccc(F)c(C(F)(F)F)c2)C1] |
| BA | 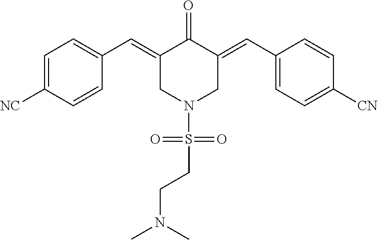 [SMILES: CN(C)CCS(=O)(=O)N1C/C(=C\c2ccc(C#N)cc2)C(=O)/C(=C/c2ccc(C#N)cc2)C1] |
| B3 | 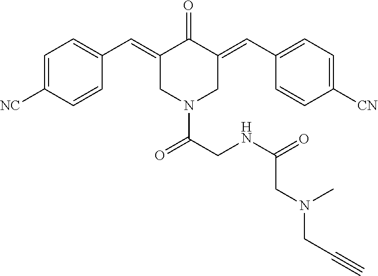 [SMILES: C#CCN(C)CC(=O)NCC(=O)N1C/C(=C\c2ccc(C#N)cc2)C(=O)/C(=C/c2ccc(C#N)cc2)C1] |
| B2 | 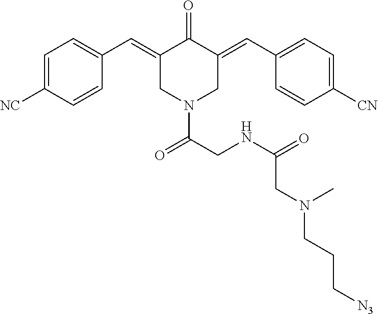 [SMILES: CN(CCCN=[N+]=[N-])CC(=O)NCC(=O)N1C/C(=C\c2ccc(C#N)cc2)C(=O)/C(=C/c2ccc(C#N)cc2)C1] |
| C1 | 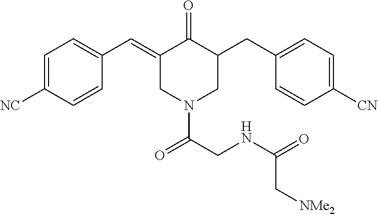 [SMILES: CN(C)CC(=O)NCC(=O)N1C/C(=C\c2ccc(C#N)cc2)C(=O)C(Cc2ccc(C#N)cc2)C1] |
| F1 | 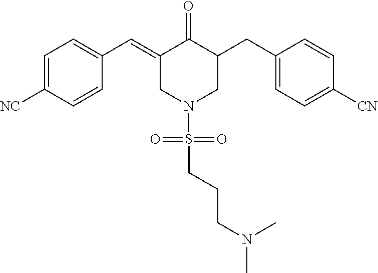 [SMILES: CN(C)CCCS(=O)(=O)N1C/C(=C\c2ccc(C#N)cc2)C(=O)C(Cc2ccc(C#N)cc2)C1] |
| B1-11 | 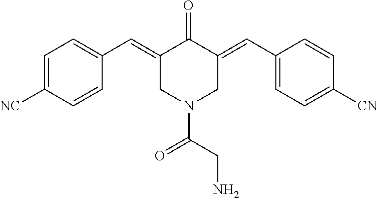 [SMILES: N#Cc1ccc(/C=C2\CN(C(=O)CN)C/C(=C\c3ccc(C#N)cc3)C2=O)cc1] |
| C1-7 | 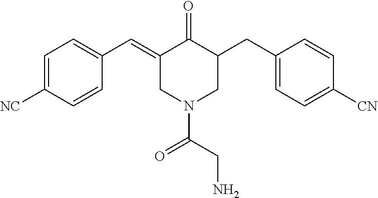 [SMILES: N#Cc1ccc(/C=C2\CN(C(=O)CN)CC(Cc3ccc(C#N)cc3)C2=O)cc1] |
| C1-8 | 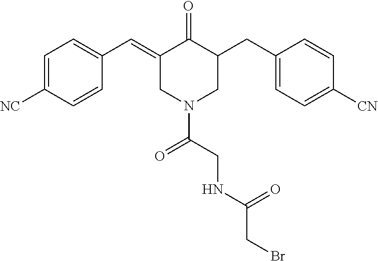 [SMILES: N#Cc1ccc(/C=C2\CN(C(=O)CNC(=O)CBr)CC(Cc3ccc(C#N)cc3)C2=O)cc1] |
| B2-7 | 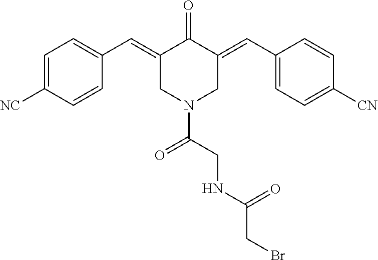 [SMILES: N#Cc1ccc(/C=C2\CN(C(=O)CNC(=O)CBr)C/C(=C\c3ccc(C#N)cc3)C2=O)cc1] |

or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof.

24. A compound represented by the structure of Formula I: 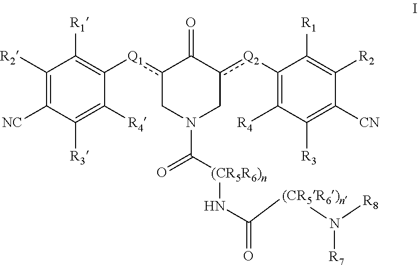
wherein
Q₁ and Q₂ are each independently, either CH or CH₂;
R₁, R₂, R₃, R₄ R₁′, R₂′, R₃′, and R₄′ are each, independently, selected from:
H, NO₂, OH, COOH, NH₂, F, Cl, Br, I, CN, R₁₃, OR₁₃, NH₂, NR₁₃R₁₄, S(O)R₁₃, S(O)₂R₁₃, —SR₁₃, SO₂NR₁₃R₁₄, NR₁₃SO₂R₁₄, C(O)R₁₃, C(O)OR₁₃, C(O)OOR₁₃, C(O)NR₁₃R₁₄, NR₁₃C(O)R₁₄, NR₁₃C(O)OR₁₄, —OCONR₁₃R₁₄, CF₃, —COCF₃, OCF₃, R₁₅-R₁₃, R₁₆-R₁₃, substituted or unsubstituted C₁-C₁₄ linear or branched alkyl group, R₁₅—COOR₁₃, substituted or unsubstituted aryl, wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₅, R₆, R₅′ and R₆′ are each, independently, selected from: H, F, Cl, Br, I, OH, R₁₅—OH, COOH, CN, C₁-C₁₀ alkyl, OR₁₃, NH₂, N(R₁₃)(R₁₄), substituted or unsubstituted (C₃-C₈) cycloalkyl, substituted or unsubstituted (C₃-C₈) heterocyclic ring having one or more heteroatoms selected from N, O and S; or R₅ and R₆ are joint to form a substituted or unsubstituted (C₃-C₈) cycloalkyl or a substituted or unsubstituted (C₃-C₈) heterocyclic ring; or R₅′ and R₆′ are joint to form a substituted or unsubstituted (C₃-C₈) cycloalkyl or a substituted or unsubstituted (C₃-C₈) heterocyclic ring; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, NR₁₃R₁₄, F, Cl, Br, I, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, S(O)R₁₃, and S(O)₂R₁₃;
R₁₃ and R₁₄ are each independently selected from: H, F, Cl, Br, I, substituted or unsubstituted linear or branched C₁-C₁₀ alkyl, substituted or unsubstituted linear or branched C₁-C₁₀ alkoxy, substituted or unsubstituted aryl, substituted or unsubstituted heteroaryl, C(O)—R₁₃, S(O)—R₁₃, S(O)₂—R₁₃, R₁₅-Ph, R₁₅-aryl, R₁₅-heteroaryl, R₁₅-R₁₃, R₁₅-R₁₆-R₁₃, —CH₂—CH═CH—C₁-C₁₀ alkyl, —CH₂—CH═CH₂, substituted or unsubstituted (C₃-C₈) cycloalkyl, substituted or unsubstituted (C₃-C₈) heterocyclic ring having one or more heteroatoms selected from N, O and S; wherein substitutions are selected from: C₁-C₁₄ linear or branched haloalkyl, C₁-C₁₄ linear or branched alkoxy, C₁-C₁₄ linear or branched alkenyl, NO₂, OH, OR₁₃, COOH, NH₂, C₁-C₁₄ alkylamino, C₁-C₁₄ dialkylamino, halogen, CN, —OCF₃, —COR₁₃, —COOR₁₃, —OCOOR₁₃, —OCONR₁₃R₁₄, —(C₁-C₈) alkylene-COOR₁₃, —SH, —SR₁₃, —(C₁-C₈) alkyl, —NR₁₃R₁₄, —CONR₁₃R₁₄, N₃, and S(O)_{q1}R₁₃;
R₁₃ and R₁₄ are each independently selected from: H, Cl, Br, I, F, OH, substituted or unsubstituted C₁-C₁₄ linear or branched alkyl group, substituted or unsubstituted (C₃-C₈) cycloalkyl, substituted or unsubstituted (C₃-C₈) heterocyclic ring having one or more heteroatoms selected from N, O and S; substituted or unsubstituted aryl, substituted or unsubstituted heteroaryl, —C(O)—C₁-C₁₄ substituted or unsubstituted linear or branched alkyl, or —S(O)₂—C₁-C₁₄ substituted or unsubstituted linear or branched alkyl, wherein substitutions are selected from C₁-C₁₄ linear or branched haloalkyl, C₁-C₁₄ linear or branched alkoxy, C₁-C₁₄ linear or branched alkenyl, C₁-C₁₄ linear or branched alkynyl, aryl, phenyl, heteroaryl, NO₂, OH, COOH, NH₂, C₁-C₁₄ alkylamino, C₁-C₁₄ dialkylamino, F, Cl, Br, I, N₃, and CN;
R₁₅ is [CH₂]ₚ
wherein p is between 1 and 10;
R₁₆ is [CH]_{q}, [C]_{q}
wherein q is between 2 and 10; and
n and n′ are each independently an integer between 1 and 15;
or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof.

25. The compound of claim 24, wherein R₇ and R₈ are each independently substituted or unsubstituted linear or branched C₁-C₁₀ alkyl, a methyl, a propyl azide or a propynyl; R₁, R₂, R₃, R₁′, R₂′, R₃′, and R₄′ are H; R₁, R₂, R₃, R₁′, R₂′, R₃′, and R₄′ are H; Q₁ is CH; O₂ is CH or CH₂; R₇ is a methyl, C₃ alkyl substituted with N₃ or CH₂—C≡CH; R₈ is a methyl; or any combination thereof.

26.-29. (canceled)

30. The compound of claim 24, represented by the following structures:

| Compound name | Structure |
|---|---|
| B1 | 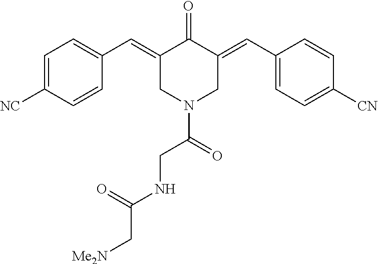 [SMILES: CN(C)CC(=O)NCC(=O)N1C/C(=C\c2ccc(C#N)cc2)C(=O)/C(=C/c2ccc(C#N)cc2)C1] |
| B3 | 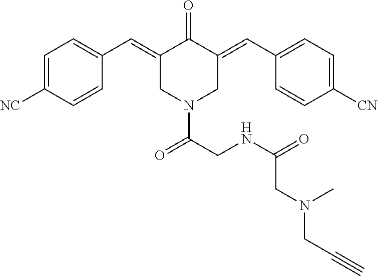 [SMILES: C#CCN(C)CC(=O)NCC(=O)N1C/C(=C\c2ccc(C#N)cc2)C(=O)/C(=C/c2ccc(C#N)cc2)C1] |
| B2 | 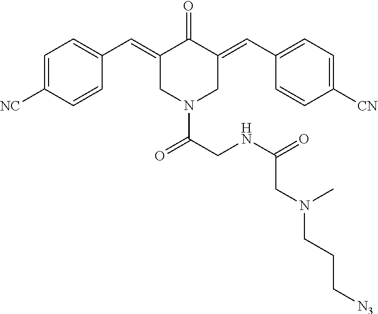 [SMILES: CN(CCCN=[N+]=[N-])CC(=O)NCC(=O)N1C/C(=C\c2ccc(C#N)cc2)C(=O)/C(=C/c2ccc(C#N)cc2)C1] |
| C1 | 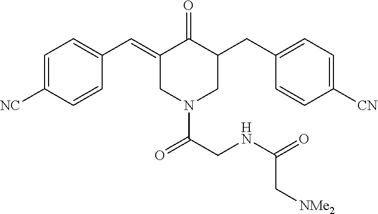 [SMILES: CN(C)CC(=O)NCC(=O)N1C/C(=C\c2ccc(C#N)cc2)C(=O)C(Cc2ccc(C#N)cc2)C1] |
| G1 | 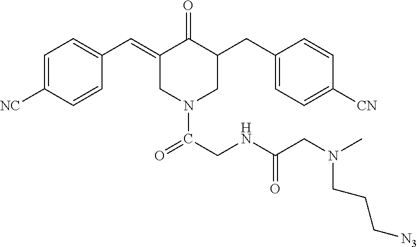 [SMILES: CN(CCCN=[N+]=[N-])CC(=O)NCC(=O)N1C/C(=C\c2ccc(C#N)cc2)C(=O)C(Cc2ccc(C#N)cc2)C1] |
| H1 | 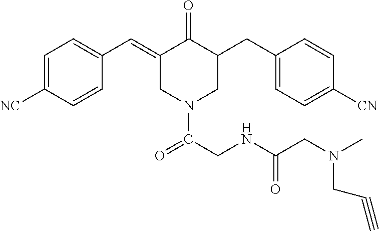 [SMILES: C#CCN(C)CC(=O)NCC(=O)N1C/C(=C\c2ccc(C#N)cc2)C(=O)C(Cc2ccc(C#N)cc2)C1] |

or geometrical isomer, optical isomer, solvate, metabolite, pharmaceutically acceptable salt, pharmaceutical product, tautomer, hydrate, N-oxide, prodrug, isotopic variant, PROTAC, polymorph, or crystal thereof.

31. The compound of any one of the preceding claims, wherein the compound is a protein degradation inhibitor, a UPS inhibitor, an autophagy modulator, a UPR inducer, wherein the compound induces proteotoxic stress and UPR by modulating protein degradation pathways and disrupting protein homeostasis, the compound induces accumulation of poly-ubiquitinated proteins in cells treated therewith, the compound disrupts autophagosomal flux in cells treated therewith, the compound induces the unfolded protein response (UPR) in cells treated therewith, or any combination thereof.

32. (canceled)

33. A pharmaceutical composition comprising the compound of claim 1 and a pharmaceutically acceptable carrier.

34. A method of treating, suppressing, reducing the severity, reducing the risk of developing or inhibiting in a subject a disease or condition selected from:
a. cancer;
b. a plasma cell disorder;
c. a non-plasma-cell hematologic malignancy;
d. a hematologic condition;
e. a SMARCB1-deficient malignancy;
f. a Post-transplant lymphoproliferative disease (PTLD);
g. a multiple myeloma;
comprising administering a compound according to claim 1 to a subject suffering from said disease or condition, under conditions effective to treat, suppress, reduce the severity, reduce the risk of developing, or inhibit said disease or condition.

35. The method of claim 34, wherein the cancer is selected from the list of:
multiple myeloma, leukemia, Alveolar rhabdomyosarcoma, Melanoma, lymphoma, Astrocytoma, Biphasic synovial sarcoma, Bladder carcinoma, Bone cancer, Breast Cancer, Cecum adenocarcinoma, Cervical cancer, CNS cancer, Colon cancer, Colorectal cancer, Duodenal adenocarcinoma, Embryonal rhabdomyosarcoma, Endometrial cancer, Epithelioid sarcoma, Fibrosarcoma, Gastric cancer, Signet ring cell gastric adenocarcinoma, Gestational choriocarcinoma, Glioblastoma, Hereditary thyroid gland medullary carcinoma, Hypopharyngeal squamous cell carcinoma, Invasive ductal carcinoma, Liposarcoma, Lung cancer, Neuroblastoma, Osteosarcoma, Ovarian cancer, Uterine cancer, Pancreatic cancer, Papillary renal cell carcinoma, Prostate cancer, Rectal adenocarcinoma, Medulloblastoma, Renal cancer, Testicular embryonal carcinoma and Tongue squamous cell carcinoma;
wherein the plasma cell disorder is Monoclonal Gammopathy of Undetermined Significance (MGUS), smoldering multiple myeloma (SMM), Asymptomatic Plasma Cell Myeloma, Multiple myeloma (MM), Waldenstrom's macroglobulinemia (WM), immunoglobulin light chain (AL) amyloidosis, POEMS syndrome, plasma cell (PC) leukemia, or Plasmacytoma;
wherein the plasma cell disorder is malignant;
wherein the Non-plasma-cell hematologic malignancy is B-cell non-Hodgkin's lymphoma (NHL) such as Mantle cell lymphoma (MCL);
wherein the hematologic conditions is AL Amyloidosis, post-transplant lymphoproliferative disease (PTLD) or combination thereof; or
wherein the PTLD is polymorphic PTLD or monomorphic PTLD or classical Hodgkin-lymphoma-type PTLD.

36. The method of claim 34, wherein the cancer is early cancer, advanced cancer, invasive cancer, metastatic cancer, drug resistant cancer or any combination thereof; wherein the subject has been previously treated with chemotherapy, immunotherapy, radiotherapy, biological therapy, surgical intervention, or any combination thereof; wherein the compound is administered in combination with an anti-cancer therapy; or any combination thereof.

37. (canceled)

38. (canceled)

39. The method of claim 38, wherein the anti-cancer therapy is chemotherapy, immunotherapy, radiotherapy, biological therapy, surgical intervention, or any combination thereof.

40. A method of suppressing, reducing or inhibiting tumor growth in a subject, comprising administering a compound according to claim 1, to a subject suffering from cancer under conditions effective to suppress, reduce or inhibit said tumor growth in said subject.

41. The method of claim 40, wherein the tumor is a solid tumor and/or a SMARCB1-deficient tumor.

42.-53. (canceled)

54. A compound represented by any one of the following structures: 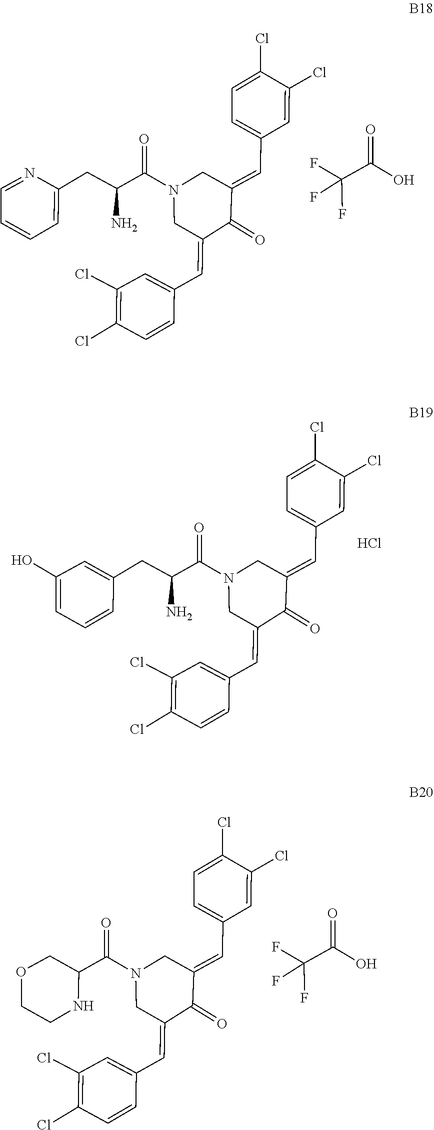 [SMILES: N[C@@H](Cc1ccccn1)C(=O)N1C/C(=C\c2ccc(Cl)c(Cl)c2)C(=O)/C(=C/c2ccc(Cl)c(Cl)c2)C1 ; O=C(O)C(F)(F)F ; N[C@@H](Cc1cccc(O)c1)C(=O)N1C/C(=C\c2ccc(Cl)c(Cl)c2)C(=O)/C(=C/c2ccc(Cl)c(Cl)c2)C1 ; Cl ; O=C1/C(=C/c2ccc(Cl)c(Cl)c2)CN(C(=O)C2COCCN2)C/C1=C\c1ccc(Cl)c(Cl)c1 ; O=C(O)C(F)(F)F] [chemical structure]
